SEQUENCE LISTING (48 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 4 - protein, 344 aa, Homo sapiens
MGFNLTFHLSYKFRLLLLLTLCLTVVGWATSNYFVGAIQEIPKAKEFMANFHKTLILGKGKTLTNEASTKKVELDNCPSVSPYLRGQSKLIFKPDLTLEEVQAENPKVSRGRYRPQECKALQRVAILVPHRNREKHLMYLLEHLHPFLQRQQLDYGIYVIHQAEGKKFNRAKLLNVGYLEALKEENWDCFIFHDVDLVPENDFNLYKCEEHPKHLVVGRNSTGYRLRYSGYFGGVTALSREQFFKVNGFSNNYWGWGGEDDDLRLRVELQRMKISRPLPEVGKYTMVFHTRDKGNEVNAERMKLLHQVSRVWRTDGLSSCSYKLVSVEHNPLYINITVDFWFGA

SEQ ID NO: 5 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 6 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 8 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 9 - protein, 112 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 10 - protein, 120 aa, Artificial Sequence
  Description of Artificial Sequence Synthetic polypeptide
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSA

SEQ ID NO: 11 - protein, 106 aa, Artificial Sequence [sequence omitted]

SEQ ID NO: 12 - protein, 122 aa, Artificial Sequence
  Description of Artificial Sequence Synthetic polypeptide
QVQLQESGPGLVKPSQTLSLTCTVSGGSISSGDYFWSWIRQLPGKGLECIGHIHNSGTTYYNPSLKSRVTISVDTSKKQFSLRLSSVTAADTAVYYCARDRGGDYYYGMDVWGQGTTVTVSS

SEQ ID NO: 13 - protein, 108 aa, Artificial Sequence
  Description of Artificial Sequence Synthetic polypeptide
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 14 - protein, 125 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 15 - protein, 10 aa, Artificial Sequence
  Description of Artificial Sequence Synthetic peptide
GFTFNTYAMN

SEQ ID NO: 16 - protein, 20 aa, Artificial Sequence
  Description of Artificial Sequence Synthetic peptide
ARIRSKYNNYATYYADSVKD

SEQ ID NO: 17 - protein, 16 aa, Artificial Sequence
  Description of Artificial Sequence Synthetic peptide
VRHGNFGNSYVSWFAY

SEQ ID NO: 18 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 19 - protein, 14 aa, Artificial Sequence
  Description of Artificial Sequence Synthetic peptide
RSSTGAVTTSNYAN

SEQ ID NO: 20 - protein, 7 aa, Artificial Sequence
  Description of Artificial Sequence Synthetic peptide
GTNKRAP

SEQ ID NO: 21 - protein, 7 aa, Artificial Sequence
  Description of Artificial Sequence Synthetic peptide
ALWYSNL

SEQ ID NO: 22 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFQGGYVSWFAHWGQGTLVTVSS

SEQ ID NO: 23 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 24 - protein, 249 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFQGGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 25 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFGGGYVSWFAWWGQGTLVTVSS

SEQ ID NO: 26 - protein, 109 aa, Artificial Sequence; identical to SEQ ID NO: 23
  Description of Artificial Sequence Synthetic polypeptide

SEQ ID NO: 27 - protein, 249 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFGGGYVSWFAWWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 28 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 29 - protein, 109 aa, Artificial Sequence; identical to SEQ ID NO: 23
  Description of Artificial Sequence Synthetic polypeptide

SEQ ID NO: 30 - protein, 249 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 31 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFQGGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 32 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFGGGYVSWFAWWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 33 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 34 - protein, 1012 aa, Artificial Sequence
  Description of Artificial Sequence Synthetic polypeptide
MGWSYIILFLVATATGVHSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 35 - protein, 1012 aa, Artificial Sequence
  Description of Artificial Sequence Synthetic polypeptide
MGWSYIILFLVATATGVHSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 36 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 37 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 38 - protein, 420 aa, Artificial Sequence
  Description of Artificial Sequence Synthetic polypeptide
MGWSYIILFLVATATGVHSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 39 - DNA, 22 bp, Artificial Sequence
  Description of Artificial Sequence Synthetic primer
gaccgacgtgtgctactattac

SEQ ID NO: 40 - DNA, 21 bp, Artificial Sequence
  Description of Artificial Sequence Synthetic primer
gaggtgcttcacgagattctt

SEQ ID NO: 41 - protein, 5 aa, Artificial Sequence
  Description of Artificial Sequence Synthetic peptide
GGGGS

SEQ ID NO: 42 - protein, 10 aa, Artificial Sequence
  Description of Artificial Sequence Synthetic peptide
GGGGSGGGGS

SEQ ID NO: 43 - protein, 15 aa, Artificial Sequence
  Description of Artificial Sequence Synthetic peptide
GGGGSGGGGSGGGGS

SEQ ID NO: 44 - protein, 20 aa, Artificial Sequence
  Description of Artificial Sequence Synthetic peptide
GGGGSGGGGSGGGGSGGGGS

SEQ ID NO: 45 - protein, 25 aa, Artificial Sequence
  Description of Artificial Sequence Synthetic peptide
GGGGSGGGGSGGGGSGGGGSGGGGS

SEQ ID NO: 46 - protein, 455 aa, Mus musculus
ASQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILNFTIPPSFADIFLSKSANLTCLVSNLATYETLSISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWKQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 47 - protein, 474 aa, Macaca fascicularis
ESAGPFKWEPSVSSPNAPLDTNEVAVGCLAQDFLPDSITFSWKFKNNSDISKGVWGFPSVLRGGKYAATSQVLLASKDVMQGTDEHVVCKVQHPNGNKEQNVPLPVVAERPPNVSVFVPPRDGFVGNPRESKLICQATGFSPRQIEVSWLRDGKQVGSGITTDRVEAEAKESGPTTFKVTSTLTVSERDWLSQSVFTCRVDHRGLTFQKNVSSVCGPNPDTAIRVFAIPPSFASIFLTKSTKLTCLVTDLATYDSVTITWTRQNGEALKTHTNISESHPNGTFSAVGEASICEDDWNSGERFRCTVTHTDLPSPLKQTISRPKGVAMHRPDVYLLPPAREQLNLRESATITCLVTGFSPADIFVQWMQRGQPLSPEKYVTSAPMPEPQAPGRYFAHSILTVSEEDWNTGETYTCVVAHEALPNRVTERTVDKSTGKPTLYNVSLVILWTTLSTFVALFVLTLLYSGIVTFIKVR

SEQ ID NO: 48 - protein, 25 aa, Artificial Sequence
  Description of Artificial Sequence Synthetic peptide
  MISC_FEATURE (1)..(25): This sequence may encompass 1-5 “Gly Gly Gly Gly Ser” repeating units
  See specification as filed for detailed description of substitutions and preferred embodiments
GGGGSGGGGSGGGGSGGGGSGGGGS

(19) United States
(12) Patent Application Publication
(10) Pub. No.: US 2023/0073926 A1
(43) Pub. Date: Mar. 9, 2023

(54) HIGHLY SIALYLATED MULTIMERIC BINDING MOLECULES

(71) Applicant: IGM Biosciences, Inc., Mountain View, CA (US)

(72) Inventors: Bruce KEYT, Hillsborough, CA (US); Ramesh BALIGA, Redwood City, CA (US); Sachi AHMED, Fremont, CA (US); Kevin CARLIN, Saratoga, CA (US); Paul HINTON, Sunnyvale, CA (US); Mark SMITH, Davis, CA (US); Avneesh SAINI, Sunnyvale, CA (US); Huyen TRAN, San Jose, CA (US); Marvin PETERSON, San Ramon, CA (US)

(21) Appl. No.: 17/758,207

(22) PCT Filed: Jan. 5, 2021
(86) PCT No.: PCT/US2021/012192

§ 371 Date: Jun. 29, 2022

Related U.S. Application Data
(60) Provisional application No. 62/957,745, filed on Jan. 6, 2020.

(51) Int. Cl. C07K 16/46; C12N 9/10; C12N 5/00; C12N 15/85

(52) U.S. Cl. CPC C07K 16/468; C12N 9/1081; C12Y 204/99001; C12N 5/00; C12N 15/85; C12N 2800/107; C07K 2317/622; A61K 39/00

(57) ABSTRACT

This disclosure provides a monoclonal population of highly sialylated multimeric binding molecules where the population includes IgM antibodies, IgM-like antibodies, or other IgM-derived binding molecules, where the population of binding molecules has a higher level of sialic acid content than is found in normal serum IgM. Also provided are methods of producing such monoclonal populations of highly sialylated multimeric binding molecules.

### CROSS-REFERENCE TO RELATED APPLICATIONS

This application claims the benefit of U.S. Provisional Patent Application Ser. No. 62/957,745, filed Jan. 6, 2020, which is incorporated herein by reference in its entirety.

### SEQUENCE LISTING

The instant application contains a Sequence Listing which has been submitted electronically in ASCII format and is hereby incorporated by reference in its entirety. The ASCII copy was created on Jan. 5, 2021, is named 028WOI-Sequence-Listing, and is 92,335 bytes in size.

### BACKGROUND

Antibodies and antibody-like molecules that can multimerize, such as IgA and IgM antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, 10,351,631, 10,400,038, 10,570,191, 10,604,559, 10,618,978, 10,689,449, and 10,787,520, U.S. Patent Application Publication Nos. US 2019-0330374, US 2019-0330360, US 2019-0338040, US 2019-0338041, US 2019-0185570, and US 2019-0002566, US 2020-0239572, and PCT Publication Nos. WO 2018/187702 and WO 2019/165340, the contents of which are incorporated herein by reference in their entireties.

The pharmacokinetics (PK) and pharmacodynamics (PD) of multivalent antibodies are complex and depend on the structure of the monoclonal antibody both translationally and post-translationally, as well as the physiological system that it targets. Moreover, different antibody classes are typically processed within a subject via different cellular and physiological systems. For example, the IgG antibody class has a serum half-life of 20 days, whereas the half-lives for IgM and IgA antibodies are only about 5-8 days (Brekke, OH., and I. Sandlie, Nature Reviews Drug Discovery 2: 52-62 (2003)).

One of the key determinants of PK of an antibody or other biotherapeutic is its level and type of glycosylation (Higel, F. et al. Eur. J. Pharm. Biopharm. 139:123-131 (2019)). Sugar moieties and their derivatives covalently linked to specific residues on an antibody can determine how they are recognized by receptors such as asialo-glycoprotein (ASGP) receptor, which in turn determines how quickly they are cleared from systemic circulation. Each IgM heavy chain constant region has five sites of asparagine-(N-)linked glycosylation, and the J-chain has one N-linked glycosylation site. Thus, a pentameric, J-chain containing IgM contains up to 51 glycan moieties, which results in a complex glycosylation profile (Hennicke, J., et al., Anal. Biochem. 539:162-166 (2017)). The complexity of glycans can make manufacture of homogenously glycosylated material difficult.

Despite the advances made in the design of multimeric antibodies, there remains a need to be able to manipulate the physical, pharmacokinetic and pharmacodynamic properties of these molecules.

### SUMMARY

Provided herein is a monoclonal population of multimeric binding molecules, each binding molecule including ten or twelve IgM-derived heavy chains, where the IgM-derived heavy chains include glycosylated IgM heavy chain constant regions each associated with a binding domain that specifically binds to a target, where each IgM heavy chain constant region includes at least one, at least two, at least three, at least four, or at least five asparagine (N)-linked glycosylation motifs, where an N-linked glycosylation motif includes the amino acid sequence N-X₁-S/T, where N is asparagine, X₁ is any amino acid except proline, and S/T is serine or threonine, where at least one, at least two, or at least three of the N-linked glycosylation motifs on each IgM heavy chain constant region are occupied by a complex glycan, and where the monoclonal population of binding molecules includes at least thirty-five (35) moles sialic acid per mole of the binding molecules.

In some embodiments, the monoclonal population of binding molecules includes at least 50, at least 60, at least 70, at least 80, at least 90, at least 100, at least 110, at least 120, at least 124, at least 130, at least 140, or at least 146 moles sialic acid per mole of binding molecule. In some embodiments, the monoclonal population of binding molecules includes at least 40, at least 45, at least 50, at least 55, at least 60, or at least 65 moles sialic acid per mole of binding molecule. In some embodiments, the monoclonal population of binding molecules includes about 40 to about 70, about 40 to about 60, about 40 to about 55, about 40 to about 50, about 50 to about 70, about 60 to about 70 moles sialic acid per mole of binding molecule.

In some embodiments, the IgM heavy chain constant regions are human IgM heavy chain constant regions or variants thereof including five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). In some embodiments, motifs N1, N2, and N3 are occupied by complex glycans.

In some embodiments, the monoclonal population of binding molecules is produced by the method of cell line modification, in vitro glycoengineering, or any combination thereof.

In some embodiments, the cell line modification includes transfecting a cell line that produces the monoclonal population of binding molecules with a gene encoding a sialyltransferase, thereby producing a modified cell line that overexpresses the sialyltransferase. In some embodiments, the sialyltransferase includes human beta-galactoside alpha-2,6-sialyltransferase 1 (ST6GAL1) (SEQ ID NO: 3). In some embodiments, the cell line modification further includes transfecting a cell line that produces the monoclonal population of binding molecules with a gene encoding a galactosyltransferase, thereby producing a modified cell line that overexpresses the galactosyltransferase. In some embodiments, the galactosyltransferase includes human beta-1,4-galactosyltransferase 4 (B4GALT4) (SEQ ID NO: 4).

In some embodiments, the in vitro glycoengineering includes contacting the monoclonal population of binding molecules with a soluble sialyltransferase and a sialic acid substrate. In some embodiments, the sialyltransferase includes a soluble variant of human beta-galactoside alpha-2,6-sialyltransferase 1 (ST6GAL1) (SEQ ID NO: 3). In some embodiments, the soluble variant of ST6GAL1 includes amino acids x to 406 of SEQ ID NO: 3, where x is an integer from 27 to 120. In some embodiments, the soluble variant of ST6GAL1 includes amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3. In some embodiments, the sialic acid substrate includes cytidine monophosphate-N-acetyl-neuraminic acid (CMP-NANA).

In some embodiments, the mass ratio of binding molecule: sialic acid substrate is about 1:4 to about 40:1. In some embodiments, the mass ratio of binding molecule: sialyltransferase is about 80:1 to about 5000:1. In some embodiments, the mass ratio of binding molecule: sialyltransferase is about 500:1. In some embodiments, the mass ratio of binding molecule: sialic acid substrate: sialyltransferase is about 500:62.5:1. In some embodiments, the mass ratio of binding molecule: sialyltransferase is about 2000:1. In some embodiments, the mass ratio of binding molecule: sialic acid substrate: sialyltransferase is about 2000:500:1. In some embodiments, the molar ratio of binding molecule: sialyltransferase is about 80:1. In some embodiments, the molar ratio of binding molecule: sialic acid substrate: sialyltransferase is about 80:500:1.

In some embodiments, the contacting of the monoclonal population of binding molecules with the soluble sialyltransferase and the sialic acid substrate comprises at least 30 minutes of contact. In some embodiments, the contacting comprises at least 1 hour, 2 hours, 3 hours, 4 hours, 5 hours, 6 hours, 7 hours, 24 hours, 36 hours, or 48 hours of contact. In some embodiments, the contacting of the monoclonal population of binding molecules with the soluble sialyltransferase and the sialic acid substrate occurs at about 2° C. to about 40° C. In some embodiments, the contacting occurs at 15° C. to about 37° C., 15° C. to about 30° C., or 15° C. to about 25° C.

In some embodiments, the in vitro glycoengineering further includes contacting the monoclonal population of binding molecules with a galactosyltransferase and a galactose substrate. In some embodiments, the galactosyltransferase includes a soluble variant of human beta-1,4-galactosyltransferase 4 (B4GALT4) (SEQ ID NO: 4). In some embodiments, the soluble variant of B4GALT4 includes amino acids x to 344 of SEQ ID NO: 4, where x is an integer from 39 to 120. In some embodiments, the soluble variant of B4GALT4 includes amino acids 120 to 344, 115 to 344, 110 to 344, 105 to 344, 100 to 344, 95 to 344, 90 to 344, 85 to 344, 80 to 344, 75 to 344, 70 to 344, 65 to 344, 60 to 344, 55 to 344, 50 to 344, 45 to 344, 40 to 344, or 39 to 344 of SEQ ID NO: 4. In some embodiments, the galactose substrate includes uridine-diphosphate-α-D-galactose (UDP-Gal). In some embodiments, the contacting with the galactosyltransferase and the galactose substrate occurs prior to or simultaneously with the contacting with the soluble sialyltransferase and sialic acid substrate.

In some embodiments, each binding molecule is multispecific, and two or more binding domains associated with the IgM heavy chain constant regions of each binding molecule specifically bind to different targets. In some embodiments, the binding domains associated with the IgM heavy chain constant regions of each binding molecule specifically bind to the same target. In some embodiments, the binding domains associated with the IgM heavy chain constant regions of each binding molecule are identical.

In some embodiments, the binding domains are antibody-derived antigen-binding domains. In some embodiments, each binding molecule is a pentameric or a hexameric IgM antibody including five or six bivalent IgM binding units, respectively, where each binding unit includes two IgM heavy chains each including a VH situated amino terminal to the variant IgM constant region, and two immunoglobulin light chains each including a light chain variable domain (VL) situated amino terminal to an immunoglobulin light chain constant region, and where the VH and VL combine to form an antigen-binding domain that specifically binds to the target. In some embodiments, each antigen-binding domain of each binding molecule binds to the same target. In some embodiments, each antigen-binding domain of each binding molecule is identical.

In some embodiments, the target is a target epitope, a target antigen, a target cell, a target organ, or a target virus.

In some embodiments, each binding molecule is pentameric and further includes a

J-chain, or functional fragment thereof, or a functional variant thereof. In some embodiments, the J-chain is a mature human J-chain including the amino acid sequence SEQ ID NO: 6 or a functional fragment thereof, or a functional variant thereof. In some embodiments, the J-chain includes an N-linked glycosylation motif N-X₁-S/T starting at amino acid positions corresponding to amino acid 49 of SEQ ID NO: 6 (motif N6).

In some embodiments, the J-chain is a functional variant J-chain including one or more single amino acid substitutions, deletions, or insertions relative to a reference J-chain identical to the variant J-chain except for the one or more single amino acid substitutions, deletions, or insertions, and where the monoclonal population of binding molecules exhibits an increased serum half-life upon administration to a subject animal relative to a reference IgM-derived binding molecule that is identical except for the one or more single amino acid substitutions, deletions, or insertions in the variant J-chain, and is administered using the same method to the same animal species. In some embodiments, the variant J-chain or functional fragment thereof includes one, two, three, or four single amino acid substitutions, deletions, or insertions relative to the reference J-chain. In some embodiments, the variant J-chain or functional fragment thereof includes an amino acid substitution at the amino acid position corresponding to amino acid Y102 of the wild-type mature human J-chain of SEQ ID NO: 6.

In some embodiments, the amino acid corresponding to Y102 of SEQ ID NO: 6 is substituted with alanine (A). In some embodiments, the J-chain includes the amino acid sequence SEQ ID NO: 7.

In some embodiments, the J-chain or fragment or variant thereof is a modified J-chain further including a heterologous moiety, where the heterologous moiety is fused or conjugated to the J-chain or fragment or variant thereof. In some embodiments, the heterologous moiety is a polypeptide fused to the J-chain or fragment or variant thereof. In some embodiments, the heterologous polypeptide is fused to the J-chain or fragment or variant thereof via a peptide linker. In some embodiments, the peptide linker includes at least 5 amino acids, but no more than 25 amino acids. In some embodiments, the peptide linker consists of GGGGSGGGGSGGGGS (SEQ ID NO: 43).

In some embodiments, the heterologous polypeptide is fused to the N-terminus of the J-chain or fragment or variant thereof or to the C-terminus of the J-chain or fragment or variant thereof. In some embodiments, heterologous moieties that can be the same or different are fused to the N-terminus and C-terminus of the J-chain or fragment or variant thereof.

In some embodiments, the heterologous polypeptide includes a binding domain. In some embodiments, the binding domain of the heterologous polypeptide is an antibody or antigen-binding fragment thereof. In some embodiments, the antigen-binding fragment is a scFv fragment. In some embodiments, the heterologous scFv fragment binds to CD3c. In some embodiments, the modified J-chain includes the amino acid sequence SEQ ID NO: 36 (V15J), SEQ ID NO: 37 (V15J*), SEQ ID NO: 38 (SP), SEQ ID NO: 31 (A-55-J*), SEQ ID NO: 32 (A-56-J*), SEQ ID NO: 33 (A-57-J*), amino acids 20-420 of SEQ ID NO: 34 (VJH), amino acids 20-420 of SEQ ID NO: 35 (VJ*H), or SEQ ID NOs: 6 or 7 fused via a peptide linker to an anti-CD3c scFv including HCDR1, HCDR2, HCDR3, LCDR1, LCDR2, and LCDR3 amino acid sequences including SEQ ID NO: 15, SEQ ID NO: 16, SEQ ID NO: 17, SEQ ID NO: 19, SEQ ID NO: 20, and SEQ ID NO: 21, respectively.

Also provided herein is a pharmaceutical composition comprising the monoclonal population of binding molecules disclosed herein and a pharmaceutically acceptable excipient.

Also provided herein is a recombinant host cell that produces the monoclonal population of binding molecules disclosed herein.

Further provided herein is a method of producing the monoclonal population of binding molecules disclosed herein, including culturing the host cell disclosed herein, and recovering the population of binding molecules.

Also provided herein is a method for producing a monoclonal population of highly sialylated multimeric binding molecules, including providing a cell line that expresses the monoclonal population of binding molecules, culturing the cell line, and recovering the monoclonal population of binding molecules, where each binding molecule includes ten or twelve IgM-derived heavy chains, where the IgM-derived heavy chains include glycosylated IgM heavy chain constant regions each associated with a binding domain that specifically binds to a target, where each IgM heavy chain constant region includes at least three, at least four, or at least five asparagine(N)-linked glycosylation motifs, where an N-linked glycosylation motif includes the amino acid sequence N-X₁-S/T, where N is asparagine, X₁ is any amino acid except proline, and S/T is serine or threonine, where on average at least one, at least two, or at least three of the N-linked glycosylation motifs on each IgM heavy chain constant region in the population are occupied by complex glycans, and where the cell line, culture conditions, recovery process, or a combination thereof is optimized to enrich for complex glycans including at least one, two, three, or four sialic acid terminal monosaccharides per glycan.

Also provided herein is a method for producing a monoclonal population of highly sialylated multimeric binding molecules, including providing a cell line that expresses the monoclonal population of binding molecules, culturing the cell line, and recovering the monoclonal population of binding molecules, where each binding molecule includes ten or twelve IgM-derived heavy chains, where the IgM-derived heavy chains include glycosylated IgM heavy chain constant regions each associated with a binding domain that specifically binds to a target, where each IgM heavy chain constant region includes at least three, at least four, or at least five asparagine(N)-linked glycosylation motifs, where an N-linked glycosylation motif includes the amino acid sequence N-X₁-S/T, where N is asparagine, X₁ is any amino acid except proline, and S/T is serine or threonine, where on average at least one, at least two, or at least three of the N-linked glycosylation motifs on each IgM heavy chain constant region in the population are occupied by complex glycans, and where the cell line, recovery process, or a combination thereof is optimized to enrich for complex glycans including at least one, two, three, or four sialic acid terminal monosaccharides per glycan.

In some embodiments, the cell line, culture conditions, recovery process, or a combination thereof is optimized to result in a monoclonal population of binding molecules including at least 10, at least 15, at least 20, at least 25, at least 30, at least 35, at least 40, at least 45, 50, at least 60, at least 70, at least 80, at least 90, at least 100, at least 110, at least 120, at least 124, at least 130, at least 140, or at least 146 moles sialic acid per mole of binding molecule. In some embodiments, the cell line, recovery process, or a combination thereof is optimized to result in a monoclonal population of binding molecules including at least 10, at least 15, at least 20, at least 25, at least 30, at least 35, at least 40, at least 45, at least 50, or at least 60 moles sialic acid per mole of binding molecule. In some embodiments, the cell line, recovery process, or a combination thereof is optimized to result in a monoclonal population of binding molecules including at least 30, at least 35, at least 40, at least 45, at least 50, or at least 60 moles sialic acid per mole of binding molecule. In some embodiments, the cell line, recovery process, or a combination thereof is optimized to result in a monoclonal population of binding molecules comprising about 40 to about 70, about 40 to about 60, about 40 to about 55, about 40 to about 50, about 50 to about 70, about 60 to about 70 moles sialic acid per mole of binding molecule.

In some embodiments, the IgM heavy chain constant regions are derived from human IgM heavy chain constant regions including five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). In some embodiments, one, two, or all three of motifs N1, N2, and N3 in the population of binding molecules are occupied by complex glycans on average.

In some embodiments, the provided cell line is modified to overexpress a sialyltransferase. In some embodiments, the sialyltransferase includes human beta-galactoside alpha-2,6-sialyltransferase 1 (ST6GAL1, SEQ ID NO: 3).

In some embodiments, the recovery process includes subjecting the monoclonal population of binding molecules to in vitro glycoengineering. In some embodiments, the in vitro glycoengineering includes contacting the monoclonal population of binding molecules with a soluble sialyltransferase and a sialic acid substrate. In some embodiments, the sialyltransferase includes a soluble variant of human beta-galactoside alpha-2,6-sialyltransferase 1 (ST6GAL1) (SEQ ID NO: 3). In some embodiments, the soluble variant of ST6GAL1 includes amino acids x to 406 of SEQ ID NO: 3, where x is an integer from 27 to 120. In some embodiments, the soluble variant of ST6GAL1 includes amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3. In some embodiments, the sialic acid substrate includes cytidine monophosphate (CMP)-N-acetyl-neuraminic acid (CMP-NANA).

In some embodiments, the mass ratio of binding molecule: sialic acid substrate is about 1:4 to about 40:1. In some embodiments, the mass ratio of binding molecule: sialyltransferase is about 80:1 to about 10000:1. In some embodiments, the mass ratio of binding molecule: sialyltransferase is about 500:1. In some embodiments, the mass ratio of binding molecule: sialic acid substrate: sialyltransferase is about 500:62.5:1. In some embodiments, the mass ratio of binding molecule: sialyltransferase is about 2000:1. In some embodiments, the mass ratio of binding molecule: sialic acid substrate: sialyltransferase is about 2000:500:1. In some embodiments, the molar ratio of binding molecule: sialyltransferase is about 80:1. In some embodiments, the molar ratio of binding molecule: sialic acid substrate: sialyltransferase is about 80:500:1.

In some embodiments, the contacting of the monoclonal population of binding molecules with the soluble sialyltransferase and the sialic acid substrate comprises at least 30 minutes of contact. In some embodiments, the contacting comprises at least 1 hour, 2 hours, 3 hours, 4 hours, 5 hours, 6 hours, 7 hours, 24 hours, 36 hours, or 48 hours of contact. In some embodiments, the contacting of the monoclonal population of binding molecules with the soluble sialyltransferase and the sialic acid substrate occurs at about 2° C. to about 40° C. In some embodiments, the contacting occurs at 15° C. to about 37° C., 15° C. to about 30° C., or 15° C. to about 25° C.

In some embodiments, in vitro glycoengineering further includes contacting the monoclonal population of binding molecules with a galactosyltransferase and a galactose substrate. In some embodiments, the galactosyltransferase includes a soluble variant of human beta-1,4-galactosyltransferase 4 (B4GALT4) (SEQ ID NO: 4). In some embodiments, the galactose substrate includes uridine-diphosphate-α-D-galactose (UDP-Gal). In some embodiments, the contacting with the galactosyltransferase and a galactose substrate occurs prior to or simultaneously with the contacting with the soluble sialyltransferase and a sialic acid substrate.

### BRIEF DESCRIPTION OF THE DRAWINGS/FIGURES

FIG. 1A shows the structure of a “simple” glycan. FIG. 1B shows an exemplary structure of an oligomannose glycan. FIG. 1C shows an exemplary structure of a complex glycan. FIG. 1D shows an exemplary structure of a hybrid glycan. Monosaccharides: darker circles=mannose; lighter circles=galactose; squares=N-acetylglucosamine; diamonds=N-acetylneuraminic acid (sialic acid or NANA); triangles=fucose. Derived from: Varki, A., and Schauer, R., Essentials of Glycobiology, 3d Edition, Chapter 8, Consortium of Glycobiology (2009).

FIG. 2A shows the structure of N-acetylneuraminic acid (sialic acid or NANA);

FIG. 2B shows the structure of cytidine monophosphate N-acetylneuraminic acid (CMP-NANA).

FIG. 3A is a space-filling model of a human IgM heavy chain, showing the positions of the five N-linked glycosylation sites. FIG. 3B shows an alignment of the human IgM heavy chain constant region amino acid sequence (allele IGHM*04, SEQ ID NO: 2) with those of mouse (GenBank: CAC20701.1, SEQ ID NO: 46) and cynomolgus monkey (amino acids 14 to 487 of GenBank: EHH62210.1, SEQ ID NO: 47). The amino acids corresponding to asparagine (N)-linked glycosylation motifs are boxed.

FIG. 4 shows the amount of sialylation of anti-CD20×CD3 IGM-A resulting from treatment with varying concentrations of truncated human α-2,6-sialyltransferase (ST6).

FIG. 5 shows in vitro sialylation of two different IgM antibodies, anti-DR5 IgM-B and anti-DR5 IgM-C.

FIG. 6 shows the pharmacokinetics of anti-CD20×CD3 IGM-A and anti-CD20×CD3 IGM-A-GEM antibodies in a mouse model.

FIG. 7 shows SNA-I lectin labeling of subclones. Cells were labelled with SNA-1 lectin conjugated to fluorescein isothiocyanate (FITC). The geomean of the signal from 488 em/530 ex measured by a cytometer is shown for each subclone.

FIG. 8A shows a reduced, denatured BioRad® CriterionTGX Stain-Free Precast gel visualized and imaged according to manufacturer's instructions, for purified proteins from fermentations carried out on the 2,6-sialyl transferase pool as well as 2 subclones (25 and 47). FIG. 8B shows western blot on the same proteins in FIG. 8A using a biotinylated SNA-I lectin. A streptavidin horseradish peroxidase fusion was used for the blot.

FIGS. 9A-9B shows fermentation comparison data from a 3-L bioreactor for anti-CD20×CD3 IGM-A producing cell lines. Two of the curves are shown for the control parental cell-line which does not have the 2,6-sialyltransferase gene and one for a production run with subclone 25 which has the 2,6-sialyltransferase gene. FIG. 9A shows the viable cell density (VCD) over the course of the run. FIG. 9B shows the viability of the cell-lines. FIG. 9C shows the titer as determined by size exclusion chromatography (SEC). FIG. 9D shows the ratio of the sialic acid measured on the purified IgM.

FIG. 10A shows a plot of the screening at the 96-well level for CHO cell clones transfected with 2,6-sialyltransferase. FIG. 10B shows a plot of the cytometry based analysis of cell surface 2,6-sialic acid levels.

FIGS. 11A and 11B show the 2,3-sialic acid and 2,6-sialic acid levels for untransfected cells, respectively. FIG. 11C compares the 2,3-sialic acid and 2,6-sialic acid levels in untransfected and transfected cells.

FIG. 12 shows T cell activation with various amounts of antibodies with a range of sialic acid levels.

FIGS. 13A-13B show a time course of sialylation of anti-CD20×CD3-IGM-A with various amounts of ST6 and CMP-NANA and at various temperatures.

FIG. 14 shows a time course of sialylation of anti-CD20×CD3-IGM-A with various amounts of ST6 and CMP-NANA and room temperature.

FIG. 15 shows a comparison of sialic acid levels and the resulting AUC_{0-∞} for various antibodies.

FIG. 16 shows the pharmacokinetics of anti-CD20×CD3 IGM-F (SA 18) and anti-CD20×CD3 IGM-F-GEM (SA 51) antibodies in a cynomolgus monkey model.

FIG. 17A shows the relative numbers of cynomolgus B cells at each time point after administration of anti-CD20×CD3-IGM-F (SA 9 or 18) or anti-CD20×CD3-IGM-F-GEM (SA 51). FIG. 17B shows the day that cynomolgus B cells began to recover after administration of anti-CD20×CD3-IGM-F (SA 9 or 18) or anti-CD20×CD3-IGM-F-GEM (SA 51).

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule, or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse B-glucuronidase.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (WH) of an antibody derived from a camelid species, or six immunoglobulin complementarity determining regions (CDRs) expressed in a fibronectin scaffold. A “binding molecule,” or “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. In other embodiments, e.g., where the binding molecule is multimeric, e.g., a dimeric IgA antibody or IgA-like antibody, a pentameric IgM antibody or IgM-like antibody, or a hexameric IgM antibody or IgM-like antibody, or any derivative thereof, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 6. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain of the amino acid sequence of SEQ ID NO: 6 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a dimer, and binding of such polymers to a target. Exemplary modified J-chains can be found, e.g., in U.S. Pat. Nos. 9,951,134, 10,400,038, and 10,618,978, and in U.S. Patent Application Publication No. US-2019-0185570, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res. 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)):

| Sequential (SEQ ID NO: 1 or SEQ ID No: 2)/KABAT numbering key for | |
|---|---|
| IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10^{⊕6} sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10^{—15} M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be, e.g., human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody, IgM-like antibody, or other IgM-derived binding molecule is able to form a multimer, e.g., a hexamer or a pentamer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.”

As used herein, the term “light chain subunit” includes amino acid sequences derived from an immunoglobulin light chain The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

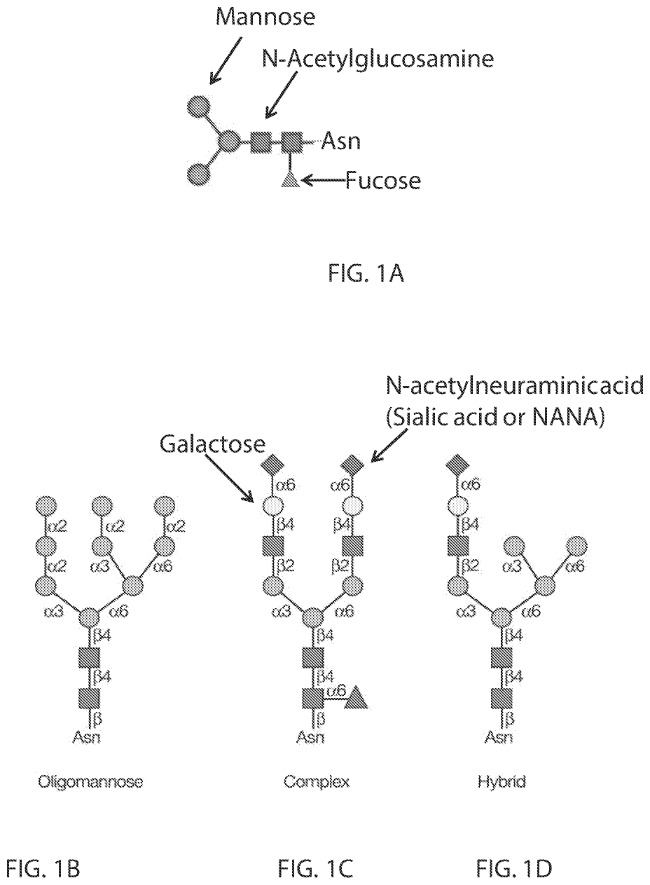

The terms “N-linked oligosaccharide” “N-linked sugar,” “N-linked glycan” or other similar or grammatical variants, denote oligosaccharide chains that are linked to a peptide backbone via an asparagine residue. All N-linked oligosaccharides have a common pentasaccharide core of Man3GlcNAc2, also called a “simple oligosaccharide,” see FIG. 1A. N-linked glycans can be generally classified into three types: (1) oligomannose, in which only mannose residues are attached to the core (FIG. 1B); (2) complex, in which “antennae” initiated by N-acetylglucosaminyltransferases (GlcNAcTs) are attached to the core (FIG. 1C); and (3) hybrid, in which only mannose residues are attached to the Manα1-6 arm of the core and one or two antennae are on the Manα1-3 arm (FIG. 1D). See, e.g., Varki, A., and Schauer, R., Essentials of Glycobiology, 3d Edition, Chapter 8, Consortium of Glycobiology (2009).

The term “glycosyltransferase” denotes an enzyme capable of transferring a monosaccharide moiety from a nucleotide sugar to an acceptor molecule such as an oligosaccharide. Examples of such glycosyltransferases include, but not limited to glucosyltransferases, mannosyltransferases, galactosyltransferases, and sialyltransferases. These enzymes are typically type II membrane proteins residing in the Golgi apparatus of cells, with the active portion of the enzyme residing in the Golgi lumen. In glycosyltransferase catalysis, the monosaccharide substrate units glucose (Glc), galactose (Gal), N-acetylglucosamine (GlcNAc), N-acetylgalactosamine (GalNAc), glucuronic acid (GlcUA), galacturonic acid (GalUA) and xylose are activated as uridine diphosphate (UDP)-α-D derivatives; arabinose is activated as a UDP-β-L derivative; mannose (Man) and fucose are activated as GDP-α-D and GDP-β-L derivatives, respectively; and sialic acid (=β-D-Neu5Ac; =Neu5Ac; =SA; =NANA) is activated as a CMP derivative of sialic acid. See, e.g., U.S. Patent Application Publication No. US 2017/0298405.

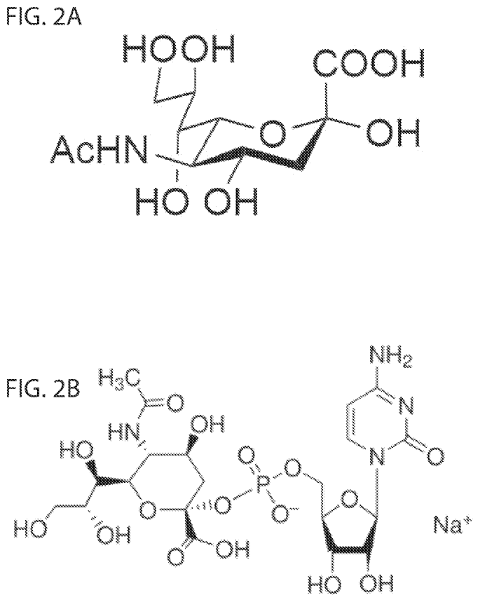

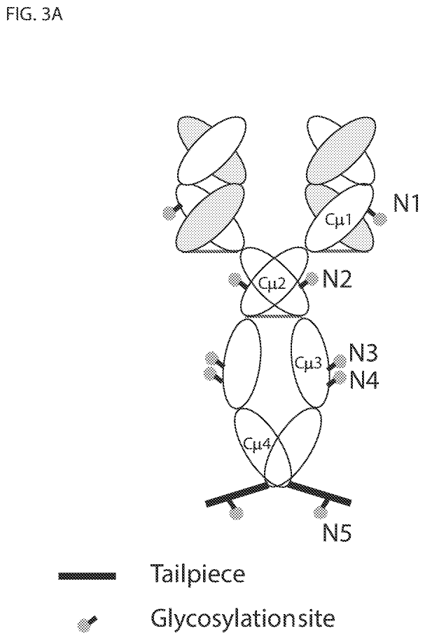

The term “sialic acid” denotes any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). FIG. 2A, see, e.g., Varki, A., and Schauer, R., Essentials of Glycobiology, 3d Edition, Chapter 14, Consortium of Glycobiology (2009).

A sialyltransferase (=“ST”) is a glycosyltransferase that catalyzes transfer of a sialic acid residue from a donor substrate to, e.g., a terminal monosaccharide acceptor group of an N-linked glycan of a glycoprotein. Mammalian sialyltransferases, including human ST species, use a common donor substrate which is cytidine-5′-monophospho-N-acetylneuraminic acid (=CMP-β-D-Neu5Ac; =CMP-Neu5Ac; =CMP-NANA; =CMP-sialic acid; =CMP-SA, FIG. 2B). Other functional equivalents are known, including, but not limited to azido-CMP-sialic acid to be used for glycan labelling by “click” chemistry. See, e.g., Moh, et al., Anal. Biochem. 584:11385 (2019). Transfer and covalent coupling of a sialic acid residue (or the functional equivalent thereof) to a receptor site is also referred to as “sialylating” and “sialylation.”

A terminal sialic acid residue can be coupled to a galactose residue by various linkages, e.g., (i) α2→3 (α2,3) linked to a galactose or (ii) α2→6 (α2,6) linked to a galactose. A sialyltransferase enzyme is generally named and classified according to its respective monosaccharide acceptor substrate and according to the position of the glycosidic bond it catalyzes. Exemplary eukaryotic sialyltransferases include (i) ST3Gal (found, e.g., in CHO cells) and (ii) ST6Gal found in human cells. Shorthand reference to “ST3” specifically encompasses the sialyltransferases catalyzing an α2,3 sialylation. Shorthand reference to “ST6” specifically encompasses the sialyltransferases catalyzing an α2,6 sialylation.

The disaccharide moiety β-D-galactosyl-1,4-N-acetyl-β-D-glucosamine (=Galβ1,4GlcNAc) is a frequent sialic acid acceptor of the antennae of N-linked glycans of glycoproteins. In addition, a terminal Galβ1,4GlcNAc moiety can be generated in certain target glycoproteins as a result of galactosyltransferase enzymatic activity, e.g., human β-1,4-galactosyltransferase 4 (=“hB4GALT4”). The enzyme β-galactoside-α2,6-sialyltransferase (=“ST6Gal”) is able to catalyze α2,6-sialylation of a terminal Galβ1,4GlcNAc acceptor moiety of a glycan or a branch of a glycan, or antenna.

The activity of ST6Gal enzymes catalyzes transfer of a Neu5Ac residue to the C6 hydroxyl group of a free galactosyl residue being part of terminal Galβ1,4GlcNAc in a glycan or an antenna of a glycan, thereby forming in the glycan a terminal sialic acid residue α2→6 linked to the galactosyl residue of the Galβ1,4GlcNAc moiety.

The wild-type polypeptide of human β-galactoside-α-2,6-sialyltransferase I (hST6Gal-I, UniProtKB/Swiss-Prot: P15907.1), is presented as SEQ ID NO: 3. Mammalian sialyltransferases share with other mammalian Golgi-resident glycosyltransferases a type II architecture with a cytoplasmic N-terminal tail, a transmembrane region, a stem region of variable length, and a C-terminal catalytic domain in the lumen of the Golgi apparatus. The cytoplasmic region of hST6GAL-1 includes amino acids 1-9 of SEQ ID NO: 3, the transmembrane region includes amino acids 10-26 of SEQ ID NO: 3, and the luminal region includes amino acids 27-406 of SEQ ID NO: 3. A soluble variant of hST6Gal-I would lack at least the transmembrane region and could further lack the N-terminal cytoplasmic region, and some portion of the luminal region, provided that the enzyme retains catalytic activity. In certain embodiments, a soluble variant of ST6GAL1 can include amino acids x to 406 of SEQ ID NO: 3, where x is an integer from 27 to 120. For example, a soluble variant of ST6GAL1 can include amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3. US Patent Application No. US 2017/0298405 reports that amino acids 90-109 of SEQ ID NO: 3 confers an additional sialidase activity to the enzyme in the presence of free CMP.

The wild-type polypeptide of human β-1,4-galactosyltransferase 4 (hB4GALT4, UniProtKB/Swiss-Prot: 060513.1) is presented as SEQ ID NO: 4. This enzyme likewise has a type II architecture with a cytoplasmic N-terminal region, a transmembrane region, a stem region of variable length, and a C-terminal catalytic region in the lumen of the Golgi apparatus. The cytoplasmic region of hB4GALT4 includes amino acids 1-12 of SEQ ID NO: 4, the transmembrane region includes amino acids 13-38 of SEQ ID NO: 4, and the luminal region includes amino acids 39-344 of SEQ ID NO: 4. A soluble variant of hB4GALT4 would lack at least the transmembrane region and could further lack the N-terminal cytoplasmic region, and some portion of the luminal region, provided that the enzyme retains catalytic activity. In certain embodiments, a soluble variant of hB4GALT4 can include amino acids x to 344 of SEQ ID NO: 4, wherein xis an integer from 39 to 120. For example, a soluble variant of hB4GALT4 can include amino acids 120 to 344, 115 to 344, 110 to 344, 105 to 344, 100 to 344, 95 to 344, 90 to 344, 85 to 344, 80 to 344, 75 to 344, 70 to 344, 65 to 344, 60 to 344, 55 to 344, 50 to 344, 45 to 344, 40 to 344, or 39 to 344 of SEQ ID NO: 4.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder and/or those prone to have the disorder.

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject. In certain embodiments the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

IgM Antibodies, IgM-Like Antibodies, Other IgM-Derived Binding Molecules, and Populations of Such Molecules

IgM is the first immunoglobulin produced by B cells in response to stimulation by antigen. Naturally-occurring IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and . CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cμ4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

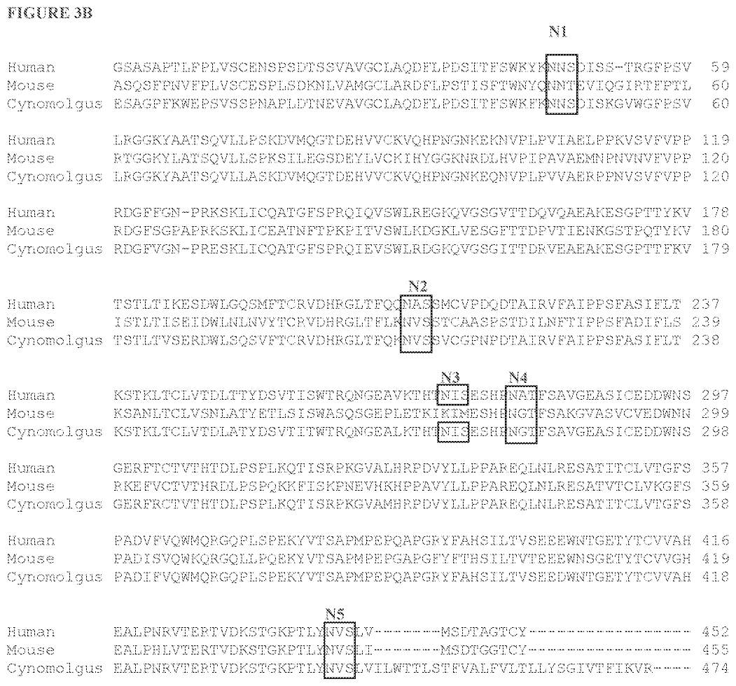

Human IgM constant regions, and also certain non-human primate IgM constant regions, as provided herein typically include five (5) naturally-occurring asparagine (N)-linked glycosylation motifs or sites. See FIGS. 3A and 3B. As used herein “an N-linked glycosylation motif” comprises or consists of the amino acid sequence N-X₁-S/T, wherein N is asparagine, X₁ is any amino acid except proline (P), and S/T is serine (S) or threonine (T). The glycan is attached to the nitrogen atom of the asparagine residue. See, e.g., Drickamer K, Taylor ME (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. See, e.g., FIG. 3B.

Studies on recombinant as well as serum-derived human IgM have shown that the N1, N2, and N3 motifs on the human IgM heavy chain are decorated predominantly, but not invariably with complex-type N-glycans, where the N4 and N5 motifs are decorated predominantly, but not invariably with oligomannose type N-glycans. See, e.g., Moh, E. S. X., et al., J. Am. Soc. Mass Spectrom. 27:1143-1155 (2016) and Hennicke, J., et al., Anal. Biochem. 539:162-166 (2017).

Each IgM heavy chain constant region can be associated with a binding domain, e.g., an antigen-binding domain, e.g., a scFv or VHH, or a subunit of an antigen-binding domain, e.g., a VH region. In certain embodiments the binding domain can be a non-antibody binding domain, e.g., a receptor ectodomain, a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. See, e.g., PCT Application Publication No. WO 2020/086745, which is incorporated herein by reference in its entirety.

Five IgM binding units can form a complex with an additional small polypeptide chain (the J-chain), or a functional fragment, variant, or derivative thereof, to form a pentameric IgM antibody or IgM-like antibody. The precursor form of the human J-chain is presented as SEQ ID NO: 5. The signal peptide extends from amino acid 1 to about amino acid 22 of SEQ ID NO: 5, and the mature human J-chain extends from about amino acid 23 to amino acid 159 of SEQ ID NO: 5. The mature human J-chain includes the amino acid sequence SEQ ID NO: 6.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody provided in this disclosure typically includes at least the Cμ4 and/or tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (μ) chain constant domain, e.g., SEQ ID NO: 1 or SEQ ID NO: 2, or a variant, derivative, or analog thereof, e.g., as provided herein.

In certain embodiments, the disclosure provides a monoclonal population of multimeric, e.g., pentameric or hexameric binding molecules, where each binding molecule includes ten or twelve IgM-derived heavy chains, and where the IgM-derived heavy chains comprise glycosylated IgM heavy chain constant regions each associated with a binding domain that specifically binds to a target. These embodiments are described in detail elsewhere in this disclosure. In certain embodiments, the disclosure provides a monoclonal population of IgM antibodies, IgM-like antibodies, or other IgM-derived binding molecules that include five or six bivalent binding units, where each binding unit includes two IgM or IgM-like heavy chain constant regions or multimerizing fragments or variants thereof, each associated with an antigen-binding domain or subunit thereof. In certain embodiments, the two IgM heavy chain constant regions included in each binding unit are human heavy chain constant regions.

Where the IgM antibody, IgM-like antibody, other IgM-derived binding molecule, or monoclonal population of multimeric binding molecules provided in this disclosure is pentameric, the IgM antibody, IgM-like antibody, other IgM-derived binding molecule, or the molecules contained in a monoclonal population of multimeric binding molecules typically further include a J-chain, or functional fragment or variant thereof. In certain embodiments the J-chain is a modified J-chain or variant thereof that further comprises one or more heterologous moieties attached to the J-chain, as described elsewhere herein. In certain embodiments the J-chain can be mutated to affect, e g , enhance, the serum half-life of the IgM antibody, IgM-like antibody, other IgM-derived binding molecule, or monoclonal population of multimeric binding molecules provided herein, as discussed elsewhere in this disclosure. In certain embodiments the J-chain can be mutated to affect glycosylation, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

Modified J-Chains

In certain embodiments, the J-chain of a pentameric IgM-derived binding molecule, e.g., an IgM antibody or IgM-like antibody as provided herein can be modified, e.g., by introduction of a heterologous moiety, or two or more heterologous moieties, e.g., polypeptides, without interfering with the ability of IgM antibody, IgM-like antibody, other IgM-derived binding molecule, or monoclonal population of multimeric binding molecules to assemble and bind to its binding target(s). See U.S. Pat. Nos. 9,951,134, 10,400,038, and 10,618,978, and U.S. Patent Application Publication No. US-2019-0185570, each of which is incorporated herein by reference in its entirety. Accordingly, an IgM antibody, IgM-like antibody, other IgM-derived binding molecule, or monoclonal population of multimeric binding molecules provided by this disclosure, including multispecific IgM or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker, e.g., a peptide linker, typically consisting of least 5 amino acids, but no more than 25 amino acids. In certain embodiments, the peptide linker consists of GGGGS (SEQ ID NO: 41), GGGGSGGGGS (SEQ ID NO: 42), GGGGSGGGGSGGGGS (SEQ ID NO: 43), GGGGSGGGGSGGGGSGGGGS (SEQ ID NO: 44), or GGGGSGGGGSGGGGSGGGGSGGGGS (SEQ ID NO: 45). In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Application Publication No. WO 2020/086745, which is incorporated herein by reference in its entirety), a stabilizing peptide that can increase the half-life of the IgM antibody, IgM-like antibody, other IgM-derived binding molecule, or monoclonal population of multimeric binding molecules, e.g., human serum albumin (HSA) or an HSA binding molecule, or a heterologous chemical moiety such as a polymer or a cytotoxin.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof, as described elsewhere herein. In certain embodiments the antigen-binding domain can be a scFv antigen-binding domain or a single-chain antigen-binding domain derived, e.g., from a camelid or condricthoid antibody. The antigen-binding domain can be introduced into the J-chain at any location that allows the binding of the antigen-binding domain to its binding target without interfering with J-chain function or the function of an associated IgM or IgA antibody. Insertion locations include but are not limited to at or near the C-terminus, at or near the N-terminus or at an internal location that, based on the three-dimensional structure of the J-chain, is accessible. In certain embodiments, the antigen-binding domain can be introduced into the mature human J-chain of SEQ ID NO: 6 between cysteine residues 92 and 101 of SEQ ID NO: 6. In a further embodiment, the antigen-binding domain can be introduced into the human J-chain of SEQ ID NO: 6 at or near a glycosylation site. In a further embodiment, the antigen-binding domain can be introduced into the human J-chain of SEQ ID NO: 6 within about 10 amino acid residues from the C-terminus, or within about 10 amino acids from the N-terminus.

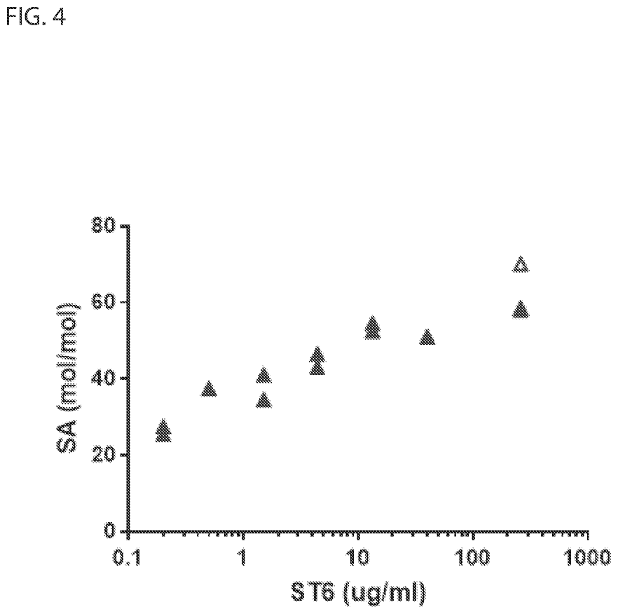

In certain embodiments, the J-chain of the IgM antibody, IgM-like antibody, or other IgM-derived binding molecule as provided herein comprises an amino acid substitution at the amino acid position corresponding to amino acid Y102 of the mature wild-type human J-chain (SEQ ID NO: 6). By “an amino acid corresponding to amino acid Y102 of the mature wild-type human J-chain” is meant the amino acid in the sequence of the J-chain of any species which is homologous to Y102 in the human J-chain. See US Patent Application Publication No. US 2020-0239572, which is incorporated herein by reference in its entirety. The position corresponding to Y102 in SEQ ID NO: 6 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 6 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the mutant J-chain. An IgM antibody, IgM-like antibody, other IgM-derived binding molecule, or monoclonal population of multimeric binding molecules comprising a mutation at the amino acid corresponding to Y102 of SEQ ID NO: 6 has an improved serum half-life when administered to an animal than a corresponding antibody, antibody-like molecule, binding molecule, or monoclonal population of binding molecules that is identical except for the substitution, and which is administered to the same species in the same manner. In certain embodiments, the amino acid corresponding to Y102 of SEQ ID NO: 6 can be substituted with any amino acid. In certain embodiments, the amino acid corresponding to Y102 of SEQ ID NO: 6 can be substituted with alanine (A), serine (S) or arginine (R). In a particular embodiment, the amino acid corresponding to Y102 of SEQ ID NO: 6 can be substituted with alanine. In a particular embodiment the J-chain or functional fragment or variant thereof is a variant human J-chain referred to herein as “J*,” and comprises the amino acid sequence SEQ ID NO: 7.

Highly Sialylated Populations of IgM-Derived Binding Molecules

This disclosure provides a monoclonal population of multimeric binding molecules, where each binding molecule comprises ten or twelve IgM-derived heavy chains, each comprising a glycosylated IgM heavy chain constant region, or multimerizing fragment thereof associated with a binding domain that specifically binds to a target. In certain embodiments, each IgM heavy chain constant region includes at least one, at least two, at least three, at least four, or at least five asparagine (N)-linked glycosylation motifs, where an N-linked glycosylation motif comprises the amino acid sequence N-X₁-S/T, where N is asparagine, X₁ is any amino acid except proline, and S/T is serine or threonine. In certain embodiments, at least one, at least two, at least three, at least four, or at least five of the N-linked glycosylation motifs on each IgM heavy chain constant region are occupied by a complex glycan as defined elsewhere herein. While a human or non-human primate IgM heavy chain constant region typically includes five N-linked glycosylation motifs N1 to N5, as noted earlier N4 and N5 are typically, but not invariably, occupied by oligomannose-type oligosaccharides as opposed to complex oligosaccharides. Thus, in certain embodiments, at least three of the N-linked glycosylation motifs (e.g., N1, N2, and N3) on each IgM heavy chain constant region are occupied by a complex glycan.

In certain embodiments, the monoclonal population of binding molecules provided by this disclosure comprises a level of sialylation that is greater than that observed or measured for IgM antibodies in normal circulation, i.e., the provided monoclonal population of binding molecules includes a non-naturally-occurring level of sialylation. As measured by the inventors (see, e.g., Example 4), the average level of sialylation for human IgM antibodies isolated from normal circulation is about 30-32 moles sialic acid per mole of IgM. Accordingly, this disclosure provides a monoclonal population of multimeric binding molecules as noted above, that includes at least thirty-three (33), at least thirty-four (34), or at least of at least thirty-five (35) moles sialic acid per mole of the binding molecules. Sialic acid residues are typically the terminal monosaccharides on complex glycans, and a single oligosaccharide glycan can include, e.g., one, two, three, or four sialic acid monosaccharides depending on the number of antennae on the oligosaccharide. In certain embodiments the provided monoclonal population of binding molecules can include higher levels of sialylation, e.g., the monoclonal population of binding molecules can include at least 40, at least 45, at least 50, at least 60, at least 70, at least 80, at least 90, at least 100, at least 110, at least 120, at least 124, at least 130, at least 140, or 146 moles sialic acid per mole of binding molecule. In some embodiments, the monoclonal population of binding molecules includes 33-146 moles sialic acid per mole of binding molecule, such as 33-130, 33-120, 33-110, 33-100, 33-90, 33-80, 33-70, 33-60, 33-50, 35-130, 35-120, 35-110, 35-100, 35-90, 35-80, 35-70, 35-60, 35-50, 45-130, 45-120, 45-110, 45-100, 45-90, 45-80, 45-70, 45-60, 45-50, 50-130, 50-120, 50-110, 50-100, 50-90, 50-80, 50-70, or 50-60 moles sialic acid per mole of binding molecule. In some embodiments, the monoclonal population of binding molecules comprises about 35 to about 40, about 35 to about 45, about 35 to about 50, about 35 to about 55, about 35 to about 60, about 35 to about 65, about 35 to about 70, about 40 to about 45, about 40 to about 50, about 40 to about 55, about 40 to about 60, about 40 to about 65, about 40 to about 70, about 45 to about 50, about 45 to about 55, about 45 to about 60, about 45 to about 65, about 45 to about 70, about 50 to about 55, about 50 to about 60, about 50 to about 65, about 50 to about 70, about 55 to about 60, about 55 to about 65, about 55 to about 70, about 60 to about 65, about 60 to about 70, or about 65 to about 70 moles sialic acid per mole of binding molecule. In some embodiments, the monoclonal population of binding molecules comprises about 40 to about 55 moles sialic acid per mole of binding molecule. As demonstrated in the examples herein, monoclonal population of binding molecules with sialic acid levels above 35 moles of sialic acid per mole of binding molecule have improved pharmacokinetic properties of the binding molecules compared to the same binding molecules with lower levels of sialic acid. For certain instances, it may be desirable to make and use a monoclonal population of binding molecules, wherein the sialic level is not at the maximum possible level, such about 40 to about 55 moles sialic acid per mole of binding molecule. Such molecules may have other desirable properties, such as a different solubility, ease of manufacturability, and/or immunogenicity.

As provided by this disclosure, each IgM-derived heavy chain in the provided population of binding molecules includes a glycosylated IgM or IgM-derived heavy chain constant region or multimerizing fragment or derivative thereof, which can be a full-length IgM heavy chain constant region, a multimerizing fragment of an IgM heavy chain constant region, or a hybrid constant region that includes at least the minimal portion of an IgM heavy chain constant region required for multimerization, associated with a binding domain, e.g., an antibody antigen-binding domain, that specifically binds to a target of interest. In certain embodiments, the IgM heavy chain constant regions are derived from human IgM heavy chain constant regions that include up to five N-linked glycosylation motifs N-Xi-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). The binding domain that binds to a target can be, e.g., an antigen-binding domain or a subunit of an antigen-binding domain, e.g., the heavy chain variable region (VH) of an antibody. This disclosure relates to binding molecules that bind to any target of interest.

The monoclonal population of binding molecules provided by this disclosure can be produced in a number of different ways, including, but not limited to, modifications to the cell line expressing the population of binding molecules, by in vitro glycoengineering of the monoclonal population of binding molecules during downstream processing, or any combination of these, or other methods.

In certain embodiments, the provided highly sialylated monoclonal population of multimeric binding molecules is produced via cell line modification. Cell line modifications to increase sialylation of a monoclonal population of binding molecules as provided by this disclosure include, without limitation, transfecting the cell line that produces the monoclonal population of binding molecules with one or more genes encoding glycosyltransferases, e.g., galactosyltransferases (to provide an acceptor residue for a sialic acid residue via an alpha-2,6 and/or alpha-2,3 linkage, see, e.g., FIGS. 1C and 1D) and/or or more sialyltransferases to produce cell lines that overexpress these enzymes (glycosyltransferase “knock-ins”), thereby improving and/or increasing the capacity of the ability of the cell line to facilitate the transfer of sialic acid monosaccharides from a CMP-NANA substrate or a derivative thereof to a compatible acceptor oligosaccharide. Other cell line modifications include the deletion or “knock-out” of sialidase enzymes normally produced by the cell line. Methods for “knocking in” various glucosyltransferases are described in the examples and are otherwise well known by persons of ordinary skill in the art. Likewise, methods for “knocking out” genes encoding, e g., sialidases in a cell line are readily available to the skilled person.

An exemplary sialyltransferase is human beta-galactoside alpha-2,6-sialyltransferase 1, also referred to as ST6GAL1 (SEQ ID NO: 3). Other sialyltransferases that can be “knocked in” include human beta-galactoside alpha-2,6-sialyltransferase-II (ST6GALII), and any of the four beta-galactoside α2-3-sialyltransferases (ST3GAL-I-IV). An exemplary galactosyltransferase is human beta-1,4-galactosyltransferase 4 (B4GALT4) (SEQ ID NO: 4).

In certain embodiments, the provided highly sialylated monoclonal population of multimeric binding molecules is produced via glycoengineering, to produce, e.g., a monoclonal population of glycoengineered IgM antibodies, IgM-like antibodies, or IgM-derived binding molecules (GEMs), e.g., via the addition of sialic acid residues to the monoclonal population of binding molecules during downstream processing. In certain embodiments, the in vitro glycoengineering comprises contacting the monoclonal population of binding molecules with a soluble sialyltransferase (or a soluble sialyltransferase attached to a solid support) and a sialic acid substrate (e.g., a substrate comprising cytidine monophosphate (CMP)-N-acetyl-neuraminic acid (CMP-NANA)), under conditions where sialic acid is transferred from CMP-NANA to a galactose residue on complex glycans on the population of binding molecules. The contacting can occur during one or more steps of protein purification, after which the soluble sialyltransferase can be removed by a subsequent purification step, or by separating the population of binding molecules from the solid support to which the enzyme is attached.

In certain embodiments the sialyltransferase variant to be used in production of GEMs can be a soluble variant of human beta-galactoside alpha-2,6-sialyltransferase 1 (ST6GAL1) (SEQ ID NO: 3). For example, the sialyltransferase can be a variant of ST6GAL1 that excludes the transmembrane region of SEQ ID NO: 3 (e.g., excluding amino acids 10 to 26 of SEQ ID NO: 3), or both the cytoplasmic and transmembrane regions of SEQ ID NO: 3 (e.g., excluding amino acids 1 to 9 of SEQ ID NO: 3 and amino acids 10 to 26 of SEQ ID NO: 3), but maintains the catalytic activity of the protein. In certain embodiments, the soluble variant of ST6GAL1 comprises amino acids x to 406 of SEQ ID NO: 3, wherein x is an integer from 27 to 120. For example the soluble variant of ST6GAL1 can include amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3. In certain embodiments the sialic acid substrate comprises cytidine monophosphate (CMP)-N-acetyl-neuraminic acid (=CMP-β-D-Neu5Ac; =CMP-Neu5Ac; =CMP-NANA; =CMP-sialic acid; =CMP-SA, FIG. 2B). Functional derivatives include but are not limited to azido-CMP-sialic acid to be used for glycan labelling by “click” chemistry.

The inventors have observed that despite the large numbers of glycans (51 for pentamers, 60 for hexamers), efficient and high-level sialylation of IgM antibodies can be carried out with low concentrations of the soluble variant of ST6GAL1, relative to the higher amounts required for glycoengineering of IgG antibodies. For example, efficient sialylation of IgM antibodies has been carried out with a mass ratio of IgM antibody to soluble sialyltransferase of about 5000:1 or 2000:1, and an IgM antibody to sialic acid substrate to soluble sialyltransferase ratio of about 5000:2500:1 or 2000:500:1 (an excess amount of the sialic acid substrate is provided). This calculates to a molar ratio of IgM antibody to sialyltransferase of about 200:1 or 80:1 or a molar ratio of IgM antibody to sialic acid substrate to sialyltransferase of about 200:2500:1 or 80:500:1. In certain embodiments the molar ratio of IgM antibody to sialyltransferase is at least about 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 175:1, or 200:1. In some embodiments, the mass ratio of binding molecule: sialyltransferase can be about 80:1 to about 5000:1. In some embodiments, the mass ratio of binding molecule: sialyltransferase can be about 80:1 to about 100:1, about 80:1 to about 250:1, about 80:1 to about 500:1, about 80:1 to about 750:1, about 80:1 to about 1000:1, about 80:1 to about 1250:1, about 80:1 to about 1500:1, about 80:1 to about 1750:1, about 80:1 to about 2000:1, about 80:1 to about 2500:1, about 80:1 to about 3000:1, about 80:1 to about 3500:1, about 80:1 to about 4000:1, about 80:1 to about 4500:1, about 250:1 to about 500:1, about 250:1 to about 750:1, about 250:1 to about 1000:1, about 250:1 to about 1250:1, about 250:1 to about 1500:1, about 250:1 to about 1750:1, about 250:1 to about 2000:1, about 250:1 to about 2500:1, about 250:1 to about 3000:1, about 250:1 to about 3500:1, about 250:1 to about 4000:1, about 250:1 to about 4500:1, about 250:1 to about 5000:1, about 500:1 to about 750:1, about 500:1 to about 1000:1, about 500:1 to about 1250:1, about 500:1 to about 1500:1, about 500:1 to about 1750:1, about 500:1 to about 2000:1, about 500:1 to about 2500:1, about 500:1 to about 3000:1, about 500:1 to about 3500:1, about 500:1 to about 4000:1, about 500:1 to about 4500:1, about 500:1 to about 5000:1, about 1000:1 to about 1250:1, about 1000:1 to about 1500:1, about 1000:1 to about 1750:1, about 1000:1 to about 2000:1, about 1000:1 to about 2500:1, about 1000:1 to about 3000:1, about 1000:1 to about 3500:1, about 1000:1 to about 4000:1, about 1000:1 to about 4500:1, about 1000:1 to about 5000:1, about 1500:1 to about 1750:1, about 1500:1 to about 2000:1, about 1500:1 to about 2500:1, about 1500:1 to about 3000:1, about 1500:1 to about 3500:1, about 1500:1 to about 4000:1, about 1500:1 to about 4500:1, about 1500:1 to about 5000:1, about 2000:1 to about 2500:1, about 2000:1 to about 3000:1, about 2000:1 to about 3500:1, about 2000:1 to about 4000:1, about 2000:1 to about 4500:1, about 2000:1 to about 5000:1, about 2500:1 to about 3000:1, about 2500:1 to about 3500:1, about 2500:1 to about 4000:1, about 2500:1 to about 4500:1, about 2500:1 to about 5000:1, about 3000:1 to about 3500:1, about 3000:1 to about 4000:1, about 3000:1 to about 4500:1, about 3000:1 to about 5000:1, about 3500:1 to about 4000:1, about 3500:1 to about 4500:1, about 3500:1 to about 5000:1, about 4000:1 to about 4500:1, or about 4000:1 to about 5000:1. This is in contrast to the much larger amounts of enzyme required for in vitro sialylation of IgG antibodies, with a recommended molar ratio of IgG antibody to sialyltransferase of 3:1. See, e.g., Malik, S., and Thomann, M., (2016) In Vitro Glycoengineering—Suitability for BioPharma manufacturing, Application Note, available custombiotech.roche.com.

The inventors have also observed that despite the large numbers of glycans (51 for pentamers, 60 for hexamers), efficient and high-level sialylation of IgM antibodies can be carried out with low concentrations of the sialic acid substrate, relative to the higher amounts required for glycoengineering of IgG antibodies. In some embodiments, the mass ratio of sialic acid substrate: sialyltransferase can be about 1:4 to about 3000:1, such as about 1:4 to about 1:1, about 1:4 to about 5:1, about 1:4 to about 50:1, about 1:4 to about 100:1, about 1:4 to about 500:1, about 1:4 to about 1000:1, about 1:4 to about 1500:1, about 1:4 to about 2000:1, about 1:4 to about 2500:1, about 1:1 to about 5:1, about 1:1 to about 10:1, about 1:1 to about 50:1, about 1:1 to about 100:1, about 1:1 to about 500:1, about 1:1 to about 1000:1, about 1:1 to about 1500:1, about 1:1 to about 2000:1, about 1:1 to about 2500:1, about 1:1 to about 3000:1, about 2:1 to about 5:1, about 2:1 to about 10:1, about 2:1 to about 50:1, about 2:1 to about 100:1, about 2:1 to about 500:1, about 2:1 to about 1000:1, about 2:1 to about 1500:1, about 2:1 to about 2000:1, about 2:1 to about 2500:1, about 2:1 to about 3000:1, about 5:1 to about 10:1, about 5:1 to about 50:1, about 5:1 to about 100:1, about 5:1 to about 500:1, about 5:1 to about 1000:1, about 5:1 to about 1500:1, about 5:1 to about 2000:1, about 5:1 to about 2500:1, about 5:1 to about 3000:1, about 10:1 to about 50:1, about 10:1 to about 100:1, about 10:1 to about 500:1, about 10:1 to about 1000:1, about 10:1 to about 1500:1, about 10:1 to about 2000:1, about 10:1 to about 2500:1, about 10:1 to about 3000:1, about 50:1 to about 100:1, about 50:1 to about 500:1, about 50:1 to about 1000:1, about 50:1 to about 1500:1, about 50:1 to about 2000:1, about 50:1 to about 2500:1, about 50:1 to about 3000:1, about 100:1 to about 500:1, about 100:1 to about 1000:1, about 100:1 to about 1500:1, about 100:1 to about 2000:1, about 100:1 to about 2500:1, about 100:1 to about 3000:1, about 500:1 to about 1000:1, about 500:1 to about 1500:1, about 500:1 to about 2000:1, about 500:1 to about 2500:1, about 500:1 to about 3000:1, about 1000:1 to about 1500:1, about 1000:1 to about 2000:1, about 1000:1 to about 2500:1, about 1000:1 to about 3000:1, about 1500:1 to about 2000:1, about 1500:1 to about 2500:1, about 1500:1 to about 3000:1, about 2000:1 to about 2500:1, about 2000:1 to about 3000:1, or about 2500:1 to about 3000:1. In some embodiments, the mass ratio of binding molecule: sialyltransferase can be about 80:1, about 100:1, about 250:1, about 500:1, about 750:1, about 1000:1, about 1250:1, about 1500:1, about 1750:1, about 2000:1, about 2500:1, about 3000:1, about 3500:1, about 4000:1, about 4500:1, or about 5000:1; and/or the mass ratio of sialic acid substrate: sialyltransferase can be about 5:1, about 10:1, about 50:1, about 100:1, about 500:1, about 1000:1, about 1500:1, about 2000:1, about 2500:1, or about 3000:1.

In some embodiments, the mass ratio of antibody: sialic acid substrate can be about 1:1 to about 40:1, such as about 1:1 to about 2:1, about 1:1 to about 4:1, about 1:1 to about 6:1, about 1:1 to about 8:1, about 1:1 to about 10:1, about 1:1 to about 15:1, about 1:1 to about 20:1, about 2:1 to about 4:1, about 2:1 to about 6:1, about 2:1 to about 8:1, about 2:1 to about 10:1, about 2:1 to about 15:1, about 2:1 to about 20:1, about 2:1 to about 40:1, about 4:1 to about 6:1, about 4:1 to about 8:1, about 4:1 to about 10:1, about 4:1 to about 15:1, about 4:1 to about 20:1, about 4:1 to about 40:1, about 6:1 to about 8:1, about 6:1 to about 10:1, about 6:1 to about 15:1, about 6:1 to about 20:1, about 6:1 to about 40:1, about 8:1 to about 10:1, about 8:1 to about 15:1, about 8:1 to about 20:1, about 8:1 to about 40:1, about 10:1 to about 15:1, about 10:1 to about 20:1, about 10:1 to about 40:1, about 15:1 to about 20:1, about 15:1 to about 40:1, or about 20:1 to about 40:1.

The inventors have further observed that efficient and high-level sialylation of IgM antibodies can be carried out at a greater range of temperatures and for longer periods of time than what was used for glycoengineering of IgG antibodies. In some embodiments, the in vitro glycoengineering comprises contacting the monoclonal population of binding molecules with a soluble sialyltransferase and a sialic acid substrate for at least 30 minutes, such as at least 45 minutes, at least 1 hour, at least 2 hours, at least 3 hours, at least 4 hours, at least 5 hours, at least 6 hours, at least 7 hours, at least 10 hours, at least 12 hours, at least 18 hours, at least 24 hours, at least 36 hours, or at least 48 hours. In some embodiments, the contacting occurs for about 30 minutes to about 48 hours, such as about 30 minutes to about 4 hours, about 30 minutes to about 5 hours, about 30 minutes to about 6 hours, about 30 minutes to about 7 hours, about 30 minutes to about 10 hours, about 30 minutes to about 12 hours, about 30 minutes to about 18 hours, about 30 minutes to about 24 hours, about 30 minutes to about 36 hours, about 2 hours to about 48 hours, about 3 hours to about 6 hours, about 3 hours to about 10 hours, about 3 hours to about 12 hours, about 3 hours to about 18 hours, about 3 hours to about 24 hours, about 3 hours to about 36 hours, about 3 hours to about 48 hours, about 4 hours to about 10 hours, about 4 hours to about 12 hours, about 4 hours to about 18 hours, about 4 hours to about 24 hours, about 4 hours to about 36 hours, about 4 hours to about 48 hours, about 5 hours to about 10 hours, about 5 hours to about 12 hours, about 5 hours to about 18 hours, about 5 hours to about 24 hours, about 5 hours to about 36 hours, about 5 hours to about 48 hours, about 7 hours to about 10 hours, about 7 hours to about 12 hours, about 7 hours to about 18 hours, about 7 hours to about 24 hours, about 7 hours to about 36 hours, about 7 hours to about 48 hours, about 10 hours to about 18 hours, about 10 hours to about 24 hours, about 10 hours to about 36 hours, about 10 hours to about 48 hours, about 12 to about 18 hours, about 12 hours to about 24 hours, about 12 hours to about 36 hours, about 12 hours to about 48 hours, about 18 hours to about 24 hours, about 18 hours to about 36 hours, about 18 hours to about 48 hours, about 24 hours to about 36 hours, about 24 hours to about 48 hours, or about 36 hours to about 48 hours,

In some embodiments, the in vitro glycoengineering comprises contacting the monoclonal population of binding molecules with a soluble sialyltransferase and a sialic acid substrate at a temperature at about 2° C. to about 40° C., such as at about 2° C. to about 37° C., 2° C. to about 30° C., 2° C. to about 25° C., 2° C. to about 22° C., 2° C. to about 20° C., 2° C. to about 10° C., about 4° C. to about 40° C., about 4° C. to about 37° C., 4° C. to about 30° C., 4° C. to about 25° C., 4° C. to about 22° C., 4° C. to about 20° C., 4° C. to about 10° C., about 10° C. to about 40° C., about 10° C. to about 37° C., 10° C. to about 30° C., 10° C. to about 25° C., 10° C. to about 22° C., 10° C. to about 20° C., about 20° C. to about 40° C., about 20° C. to about 37° C., 20° C. to about 30° C., 20° C. to about 25° C., 20° C. to about 22° C., about 22° C. to about 40° C., about 22° C. to about 37° C., 22° C. to about 30° C., 22° C. to about 25° C., about 25° C. to about 40° C., about 25° C. to about 37° C., 25° C. to about 30° C., about 30° C. to about 40° C., or about 30° C. to about 37° C. In some embodiments, the in vitro glycoengineering comprises contacting the monoclonal population of binding molecules with a soluble sialyltransferase and a sialic acid substrate at a temperature at about 15° C. to about 25° C.

In certain embodiments, in vitro sialylation can be enhanced by ensuring a sufficient number of galactose acceptor residues are present on the complex glycans of the provided monoclonal population of IgM, IgM-like, or IgM-derived binding molecules. ST6GAL1 transfers a sialic acid monosaccharide from CMP-NANA to a galactose acceptor residue on the molecule's glycans via an α-2,6 linkage. To insure a sufficient number of acceptor galactose residues on the glycans present in the monoclonal population of binding molecules, production of GEMs can further include contacting the monoclonal population of binding molecules with a galactosyltransferase, e.g., a soluble variant of beta-1,4-galactosyltransferase 4 (B4GALT4) (SEQ ID NO: 4), and a galactose substrate, e.g., uridine-diphosphate-α-D-galactose (UDP-Gal), either prior to or simultaneously with the contacting with the sialyltransferase and sialic acid substrate. For example, the sialyltransferase can be a variant of B4GALT4 that excludes the transmembrane region of SEQ ID NO: 4 (e.g., excluding amino acids 13 to 38 of SEQ ID NO: 4), or both the cytoplasmic and transmembrane regions of SEQ ID NO: 4 (e.g., excluding amino acids 1 to 12 of SEQ ID NO: 4 and amino acids 13 to 38 of SEQ ID NO: 4), but maintains the catalytic activity of the protein. In certain embodiments the soluble variant of B4GALT4 comprises amino acids x to 344 of SEQ ID NO: 4, wherein x is an integer from 39 to 120. For example, the soluble variant of B4GALT4 comprises amino acids 120 to 344, 115 to 344, 110 to 344, 105 to 344, 100 to 344, 95 to 344, 90 to 344, 85 to 344, 80 to 344, 75 to 344, 70 to 344, 65 to 344, 60 to 344, 55 to 344, 50 to 344, 45 to 344, 40 to 344, or 39 to 344 of SEQ ID NO: 4. In certain embodiments the galactose substrate comprises UDP-Gal.

IgM heavy chain constant regions in the provided monoclonal population of binding molecules are each associated with a binding domain or subunit thereof, e.g., an antibody antigen-binding domain, e.g., a scFv, a VHH or the VH subunit of an antibody antigen-binding domain, where the binding domain specifically binds to a target of interest. In certain embodiments, the target is a target epitope, a target antigen, a target cell, a target organ, or a target virus. Targets can include, without limitation, tumor antigens, other oncologic targets, immuno-oncologic targets such as immune checkpoint inhibitors, infectious disease targets, such as viral antigens expressed on the surface of infected cells, target antigens involved in blood-brain-barrier transport, target antigens involved in neurodegenerative diseases and neuroinflammatory diseases, and any combination thereof. Exemplary targets and binding domains that bind to such targets are provided elsewhere herein, and can be found in, e.g., U.S. Patent Application Publication Nos. US 2019-0330360, US 2019-0338040, US 2019-0338041, US 2019-0330374, US 2019-0185570, US 2019-0338031, or US 2020-0239572, in PCT Publication Nos. WO 2018/017888, WO 2018/017889, WO 2018/017761, WO 2018/017763, or WO 2018/187702, and WO 2019/165340, or in U.S. Pat. Nos. 9,951,134, 9,938,347, 8,377,435, 9,458,241, 9,409,976, 10,400,038, 10,351,631, 10,570,191, 10,604,559, 10,618,978, 10,689,449, or 10,787,520. Each of these applications and/or patents are incorporated herein by reference in their entireties.

In certain embodiments, the provided population of multimeric binding molecules is multispecific, e.g., bispecific, trispecific, or tetraspecific, where two or more binding domains associated with the IgM heavy chain constant regions of each binding molecule specifically bind to different targets. In certain embodiments, the binding domains of the provided population of multimeric binding molecules all specifically bind to the same target. In certain embodiments, the binding domains of the provided population of multimeric binding molecules are identical. In such cases the population of multimeric binding molecules can still be bispecific, if, for example, a binding domain with a different specificity is part of a modified J-chain as described elsewhere herein. In certain embodiments, the binding domains are antibody-derived antigen-binding domains, e.g., a scFv associated with the IgM heavy chain constant regions or a VH subunit of an antibody binding domain associated with the IgM heavy chain constant regions.

In certain embodiments, each binding molecule is a pentameric or a hexameric IgM antibody comprising five or six bivalent IgM binding units, respectively, wherein each binding unit comprises two IgM heavy chains each comprising a VH situated amino terminal to the variant IgM constant region, and two immunoglobulin light chains each comprising a light chain variable domain (VL) situated amino terminal to an immunoglobulin light chain constant region, and wherein the VH and VL combine to form an antigen-binding domain that specifically binds to the target. In certain embodiments each antigen-binding domain of each binding molecule binds to the same target. In certain embodiments, each antigen-binding domain of each binding molecule is identical.

In certain embodiments, the target is a tumor-specific antigen, i.e., a target antigen that is largely expressed only on tumor or cancer cells, or that may be expressed only at undetectable levels in normal healthy cells of an adult. In certain embodiments, the target is a tumor-associated antigen, i.e., a target antigen that is expressed on both healthy and cancerous cells but is expressed at a much higher density on cancerous cells than on normal healthy cells. Exemplary tumor-specific or tumor-associated antigens include, without limitation, B-cell maturation antigen (BCMA), CD19, CD20, epidermal growth factor receptor (EGFR), human epidermal growth factor receptor 2 (HER2, also called ErbB2), HERS (ErbB3), receptor tyrosine-protein kinase ErbB4, cytotoxic T-lymphocyte antigen 4 (CTLA4), programmed cell death protein 1 (PD-1), Programmed death-ligand 1 (PD-L1), vascular endothelial growth factor (VEGF), VEGF receptor-1 (VEGFR1), VEGFR2, CD52, CD30, prostate-specific membrane antigen (PSMA), CD38, ganglioside GD2, self-ligand receptor of the signaling lymphocytic activation molecule family member 7 (SLAMF7), platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), and CD33. The skilled person will understand that these target antigens appear in the literature by a number of different names, but that these therapeutic targets can be easily identified using databases available online, e.g., EXPASY_dot_org.

Other tumor associated or tumor-specific antigens include, without limitation: DLL4, Notch1, Notch2, Notch3, Notch4, JAG1, JAG2, c-Met, IGF-1R, Patched, Hedgehog family polypeptides, WNT family polypeptides, FZD1, FZD2, FZD3, FZD4, FZD5, FZD6, FZD7, FZD8, FZD9, FZD10, LRP5, LRP6, IL-6, TNFalpha, IL-23, IL-17, CD80, CD86, CD3, CEA, Muc16, PSCA, CD44, c-Kit, DDR1, DDR2, RSPO1, RSPO2, RSPO3, RSPO4, BMP family polypeptides, BMPR1a, BMPR1b, or a TNF receptor superfamily protein such as TNFR1 (DR1), TNFR2, TNFR1/2, CD40 (p50), Fas (CD95, Apo1, DR2), CD30, 4-1BB (CD137, ILA), TRAILR1 (DR4, Apo2), DR5 (TRAILR2), TRAILR3 (DcR1), TRAILR4 (DcR2), OPG (OCIF), TWEAKR (FN14), LIGHTR (HVEM), DcR3, DR3, EDAR, and XEDAR.

In certain embodiments, the monoclonal population of multimeric binding molecules comprises a population of pentameric or hexameric IgM antibodies, IgM-like antibodies, or other IgM-derived binding molecules that each include five or six bivalent IgM binding units, respectively. According to certain embodiments, each binding unit includes two IgM heavy chains as described herein, each having a VH situated amino terminal to the variant IgM constant region, and two immunoglobulin light chains each having a light chain variable domain (VL) situated amino terminal to an immunoglobulin light chain constant region, e.g., a kappa or lambda constant region. The provided VH and VL combine to form an antigen-binding domain that specifically binds to the target of interest. In certain embodiments, the five or six IgM binding units are identical.

In those embodiments where the population of multimeric IgM antibodies, IgM-like antibodies, or IgM-derived binding molecules is pentameric, each antibody or binding molecule can further include a J-chain, or functional fragment thereof, or a functional variant thereof, as described elsewhere herein. For example, the J-chain can be a mature human J-chain that includes the amino acid sequence SEQ ID NO: 6 or a functional fragment thereof, or a functional variant thereof. As persons of ordinary skill in the art will recognize, “a functional fragment” or a “functional variant” in this context includes those fragments and variants that can associate with IgM binding units, e.g., IgM heavy chain constant regions, to form a pentameric IgM antibody, IgM-like antibody, or IgM-derived binding molecule.

In certain embodiments, the J-chain of a pentameric IgM-derived binding molecule, e.g., an IgM antibody, IgM-like antibody, or other IgM-derived binding molecule as provided herein is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

As described in detail elsewhere herein, in certain embodiments the variant J-chain or functional fragment thereof of a pentameric IgM-derived binding molecule, e.g., an IgM antibody, IgM-like antibody, or other IgM-derived binding molecule as provided herein comprises an amino acid substitution at the amino acid position corresponding to amino acid Y102 of the wild-type mature human J-chain (SEQ ID NO: 6). Y102 can be substituted with any amino acid, for example alanine. In certain embodiments the variant human J-chain can include the amino acid sequence SEQ ID NO: 7. A J-chain having the amino acid sequence of SEQ ID NO: 7 can in some instances be referred to as “J*.”

The J-chain or fragment of a pentameric IgM-derived binding molecule, e.g., an IgM antibody, IgM-like antibody, or other IgM-derived binding molecule as provided herein, having either a variant or wild type amino acid sequence, can be a “modified J-chain” that further include a heterologous moiety, wherein the heterologous moiety is fused or conjugated to the J-chain or fragment or variant thereof. Exemplary, but non-limiting heterologous moieties are provided, e.g., in U.S. Pat. Nos. 9,951,134 and 10,618,978, and in U.S. Patent Application Publication No. 2019/0185570, which are incorporated herein by reference. In certain embodiments, the heterologous moiety is a polypeptide fused to or within the J-chain or fragment or variant thereof. The heterologous polypeptide can in some instances be fused to or within the J-chain or fragment or variant thereof via a peptide linker. Any suitable linker can be used, for example the peptide linker can include at least 5 amino acids, at least ten amino acids, and least 20 amino acids, at least 30 amino acids or more, and so on. In certain embodiments the peptide linker includes no more than 25 amino acids. In certain embodiments the peptide linker can consist of 5 amino acids, 10 amino acids, 15 amino acids, 20 amino acids, or 25 amino acids. In certain embodiments the peptide linker comprises glycines and serines, e.g., (GGGGS)n (SEQ ID NO: 48), where N can be 1, 2, 3, 4, 5, or more. In certain embodiments, the peptide linker consists of GGGGS (SEQ ID NO: 41), GGGGSGGGGS (SEQ ID NO: 42), GGGGSGGGGSGGGGS (SEQ ID NO: 43), GGGGSGGGGSGGGGSGGGGS (SEQ ID NO: 44), or GGGGSGGGGSGGGGSGGGGSGGGGS (SEQ ID NO: 45). In certain embodiments, the heterologous polypeptide can be fused to the N-terminus of the J-chain or fragment or variant thereof, the C-terminus of the J-chain or fragment or variant thereof, or to both the N-terminus and C-terminus of the J-chain or fragment or variant thereof. In certain embodiments the heterologous polypeptide can be fused internally within the J-chain. In certain embodiments, the heterologous polypeptide can be a binding domain, e.g., an antigen binding domain. For example, the heterologous polypeptide can be an antibody, a subunit of an antibody, or an antigen-binding fragment of an antibody, e.g., a scFv fragment. In certain embodiments, the binding domain, e.g., scFv fragment can bind to an effector cell, e.g., a T cell or an NK cell. In certain embodiments, the binding domain, e.g., scFv fragment can specifically bind to CD3 on cytotoxic T cells, e.g., to CD3ε. In certain specific embodiments, the modified J-chain of a pentameric IgM-derived binding molecule as provided herein comprises amino acids sequence SEQ ID NO: 36 (V15J) or SEQ ID NO: 37 (V15J*), or a J-chain comprising an anti-CD3ε scFv antigen-binding domain comprising the six complementarity-determining region of murine antibody SP34 (VH=SEQ ID NO: 14, VL=SEQ ID NO: 18), the VHCDR1, VHCDR2, VHCDR3, VLCDR1, VLCDR2, and VLCDR3 amino acid sequences SEQ ID NO: 15, SEQ ID NO: 16, SEQ ID NO: 17, SEQ ID NO: 19, SEQ ID NO: 20, and SEQ ID NO: 21, respectively, e.g., the modified J-chain SJ*, comprising the amino acid sequence SEQ ID NO: 39. Other humanized SP35 antibodies include the VH and VL or scFv sequences of A-55 (SEQ ID NOs 22, 23, and 24, respectively, W02018208864), A-56 (SEQ ID NOs 25, 26, and 27, respectively, WO2018208864), or A-57 (SEQ ID NOs 28, 29, and 30, respectively, WO2018208864), incorporated into the modified J-chains A-55-J* (SEQ ID NO: 31), A-56-J* (SEQ ID NO: 32), and A-57-J* (SEQ ID NO: 33). In certain embodiments a modified J-chain as provided herein can further include an additional heterologous moiety attached, e.g., on the opposite end of the J-chain from the anti-CD3ε scFv binding domain. For example, the modified J-chain can further include the human serum albumin protein. Examples include, but are not limited to, VJH (SEQ ID NO: 34) and VJ*H (SEQ ID NO: 35).

IgM-Derived Binding Molecules with Enhanced Serum Half-Life

A monoclonal population of highly sialylated IgM antibodies, IgM-like antibodies, or IgM-derived binding molecules as provided herein can be further engineered to have enhanced serum half-life. Exemplary IgM heavy chain constant region mutations that can enhance serum half-life of an IgM-derived binding molecule are disclosed in US Patent Application Publication No. US 2020-0239572, which is incorporated by reference herein in its entirety. For example, variant IgM heavy chain constant regions of the population of highly sialylated IgM antibodies, IgM-like antibodies, or IgM-derived binding molecules as provided herein can include an amino acid substitution at an amino acid position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

Wild-type J-chains typically include one N-linked glycosylation site. In certain embodiments, a variant J-chain or functional fragment thereof of a pentameric IgM-derived binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 6) or J* (SEQ ID NO: 7), wherein N is asparagine, X₁ is any amino acid except proline, and S/T is serine or threonine, and wherein the mutation prevents glycosylation at that motif. As demonstrated in US Patent Application Publication No. US 2020-0239572, mutations preventing glycosylation at this site can result in the population of IgM-derived binding molecules, e.g., an IgM antibodies, IgM-like antibodies, or other IgM-derived binding molecules as provided herein, exhibiting an increased serum half-life upon administration to a subject animal relative to a reference IgM-derived binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived binding molecule as provided herein can include an amino acid substitution at the amino acid position corresponding to amino acid N49 or amino acid S51 SEQ ID NO: 6 or SEQ ID NO: 7, provided that the amino acid corresponding to S51 is not substituted with threonine (T), or wherein the variant J-chain comprises amino acid substitutions at the amino acid positions corresponding to both amino acids N49 and S51 of SEQ ID NO: 6 or SEQ ID NO: 7. In certain embodiments, the position corresponding to N49 of SEQ ID NO: 6 or SEQ ID NO: 7 is substituted with any amino acid, e.g., alanine (A), glycine (G), threonine (T), serine (S) or aspartic acid (D). In a particular embodiment, the position corresponding to N49 of SEQ ID NO: 6 or SEQ ID NO: 7 can be substituted with alanine (A). In another particular embodiment, the position corresponding to N49 of SEQ ID NO: 6 or SEQ ID NO: 7 can be substituted with aspartic acid (D).

Variant Human IgM Constant Regions with Reduced CDC Activity

A monoclonal population of IgM-derived binding molecules, e.g., IgM antibodies, IgM-like antibodies, or other IgM-derived binding molecules as provided herein, can be engineered to exhibit reduced complement-dependent cytotoxicity (CDC) activity to cells in the presence of complement, relative to a reference population of IgM antibodies or IgM-like antibodies with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 22 or SEQ ID NO: 23. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 22 or SEQ ID NO: 23 with aspartic acid.

Host Cells

In certain embodiments, this disclosure provides a host cell that is capable of producing the highly sialylated monoclonal population of binding molecules as provided herein. In certain aspects such a host cell overexpresses ST6GAL1 and/or B4GALT4. The disclosure also provides a method of producing the monoclonal population of binding molecules as provided herein, where the method comprises culturing the provided host cell, and recovering the population of binding molecules.

Methods for Producing a Highly Sialylated Population of IgM Antibodies, IgM-Like Antibodies, or IgM-Derived Binding Molecules

This disclosure further provides a method for producing a monoclonal population of highly sialylated multimeric binding molecules as described in detail in this disclosure, where the method includes: providing a cell line that expresses the monoclonal population of binding molecules, culturing the cell line, and recovering the monoclonal population of binding molecules. In certain embodiments, each binding molecule comprises ten or twelve IgM-derived heavy chains, wherein the IgM-derived heavy chains comprise glycosylated IgM heavy chain constant regions, or multimerizing fragments thereof, each associated with a binding domain that specifically binds to a target, wherein each IgM heavy chain constant region comprises at least one, at least two, at least three, at least four, or at least five asparagine(N)-linked glycosylation motifs, wherein and wherein an N-linked glycosylation motif comprises the amino acid sequence N-X₁-S/T, where N is asparagine, X₁ is any amino acid except proline, and S/T is serine or threonine. As provided herein, on average at least one, at least two, or at least three of the N-linked glycosylation motifs on each IgM heavy chain constant region in the population is/are occupied by complex glycans, and wherein the cell line, culture conditions, recovery process, or a combination thereof is optimized to enrich for complex glycans comprising at least one, two, at least three, or four sialic acid terminal monosaccharides per glycan.

In certain embodiments, the cell line, culture conditions, recovery process, or a combination thereof can be optimized according to the provided method to result in a monoclonal population of binding molecules comprising at least 10, at least 15, at least 20, at least 25, at least 30, at least 35, at least 40, at least 45, at least 50, at least 60, at least 70, at least 80, at least 90, at least 100, at least 110, at least 120, at least 124, at least 130, at least 140, or at least 146 moles sialic acid per mole of binding molecule. In certain embodiments, the cell line, recovery process, or a combination thereof can be optimized according to the provided method to result in a monoclonal population of binding molecules comprising at least 35, at least 40, at least 45, at least 50, or at least 60 moles sialic acid per mole of binding molecule. In some embodiments, the monoclonal population of binding molecules comprises about 35 to about 40, about 35 to about 45, about 35 to about 50, about 35 to about 55, about 35 to about 60, about 35 to about 65, about 35 to about 70, about 40 to about 45, about 40 to about 50, about 40 to about 55, about 40 to about 60, about 40 to about 65, about 40 to about 70, about 45 to about 50, about 45 to about 55, about 45 to about 60, about 45 to about 65, about 45 to about 70, about 50 to about 55, about 50 to about 60, about 50 to about 65, about 50 to about 70, about 55 to about 60, about 55 to about 65, about 55 to about 70, about 60 to about 65, about 60 to about 70, or about 65 to about 70 moles sialic acid per mole of binding molecule. In some embodiments, the monoclonal population of binding molecules comprises about 40 to about 55 moles sialic acid per mole of binding molecule. According to the provided method, the IgM heavy chain constant regions can be derived from human IgM heavy chain constant regions comprising five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). In certain embodiments, on average one, two, or all three of motifs N1, N2, and N3 in the population of binding molecules are occupied by complex glycans that can be sialylated by the provided method.

In certain embodiments, the cell line cultured according to provided method is modified to overexpress a sialyltransferase. In certain embodiments, the overexpressed sialyltransferase is a 2,6-sialyl transferase. In certain embodiments, the overexpressed sialyltransferase is human beta-galactoside alpha-2,6-sialyltransferase 1 (ST6GAL1). In certain embodiments, the overexpressed sialyltransferase is a 2,3-sialyl transferase. The cell line cultured according to provided method can also be modified to overexpress a galactosyltransferase. In certain embodiments the overexpressed galactosyltransferase is human beta-1,4-galactosyltransferase 4 (B4GALT4). The cell line cultured according to provided method can also be modified to overexpress an UDP-GlcNAc 2-Epimerase/ManNAc Kinase enzyme (GNE), such as GNE comprising an 8263 or 8266 mutation, such as a Q, W, or L mutation; an α-mannosidase II; an N-Acetylglucosaminyltransferase-II (GNT-II); an N-Acetylglucosaminyltransferase-IV (GNT-IV); an N-Acetylglucosaminyltransferase-V (GNT-V); a CMP-sialic acid synthase (CMP-SAS), a CMP-sialic acid transporter (CMP-SAT), or any combination thereof. In certain embodiments the cell line cultured according to provided method can also be modified to block expression of certain sialidases. In certain embodiments the cell line cultured according to provided method can also be modified to block expression of a neuraminidase.

In certain embodiments of the provided method, the recovery process includes subjecting the monoclonal population of multimeric binding molecules to glycoengineering during downstream processing, to produce, e.g., a monoclonal population of glycoengineered IgM antibodies, IgM-like antibodies, or IgM-derived binding molecules, or “GEMs.” In certain embodiments the GEMs are highly sialylated, e.g., possessing at least 35 moles sialic acid per mole of binding molecule. Production of GEMs is described in detail elsewhere herein, and any and all aspects of the production of GEMs can be included in the provided method. In certain embodiments, the production of GEMs includes contacting the monoclonal population of binding molecules with a soluble sialyltransferase and a sialic acid substrate. In certain embodiments, the soluble sialyltransferase can be a soluble variant of human beta-galactoside alpha-2,6-sialyltransferase 1 (ST6GAL1) (SEQ ID NO: 3). In certain embodiments the soluble variant of ST6GAL1 includes amino acids x to 406 of SEQ ID NO: 3, wherein x is an integer from 27 to 120. For example, the soluble variant of ST6GAL1 can include amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3. In certain embodiments, the sialic acid substrate can include cytidine monophosphate (CMP)-N-acetyl-neuraminic acid (CMP-NANA), or a derivative thereof.

As described elsewhere herein, the inventors have discovered that the production of highly sialylated IgM antibodies, IgM-like antibodies, or IgM-derived binding molecules requires much less enzyme than a comparable method for sialylating IgG. For example, the mass ratio of binding molecule: sialyltransferase can be about 80:1 to about 5000:1. In some embodiments, the mass ratio of binding molecule: sialyltransferase can be about 80:1 to about 100:1, about 80:1 to about 250:1, about 80:1 to about 500:1, about 80:1 to about 750:1, about 80:1 to about 1000:1, about 80:1 to about 1250:1, about 80:1 to about 1500:1, about 80:1 to about 1750:1, about 80:1 to about 2000:1, about 80:1 to about 2500:1, about 80:1 to about 3000:1, about 80:1 to about 3500:1, about 80:1 to about 4000:1, about 80:1 to about 4500:1, about 80:1 to about 5000:1, about 250:1 to about 500:1, about 250:1 to about 750:1, about 250:1 to about 1000:1, about 250:1 to about 1250:1, about 250:1 to about 1500:1, about 250:1 to about 1750:1, about 250:1 to about 2000:1, about 250:1 to about 2500:1, about 250:1 to about 3000:1, about 250:1 to about 3500:1, about 250:1 to about 4000:1, about 250:1 to about 4500:1, about 250:1 to about 5000:1, about 500:1 to about 750:1, about 500:1 to about 1000:1, about 500:1 to about 1250:1, about 500:1 to about 1500:1, about 500:1 to about 1750:1, about 500:1 to about 2000:1, about 500:1 to about 2500:1, about 500:1 to about 3000:1, about 500:1 to about 3500:1, about 500:1 to about 4000:1, about 500:1 to about 4500:1, about 500:1 to about 5000:1, about 1000:1 to about 1250:1, about 1000:1 to about 1500:1, about 1000:1 to about 1750:1, about 1000:1 to about 2000:1, about 1000:1 to about 2500:1, about 1000:1 to about 3000:1, about 1000:1 to about 3500:1, about 1000:1 to about 4000:1, about 1000:1 to about 4500:1, about 1000:1 to about 5000:1, about 1500:1 to about 1750:1, about 1500:1 to about 2000:1, about 1500:1 to about 2500:1, about 1500:1 to about 3000:1, about 1500:1 to about 3500:1, about 1500:1 to about 4000:1, about 1500:1 to about 4500:1, about 1500:1 to about 5000:1, about 2000:1 to about 2500:1, about 2000:1 to about 3000:1, about 2000:1 to about 3500:1, about 2000:1 to about 4000:1, about 2000:1 to about 4500:1, about 2000:1 to about 5000:1, about 2500:1 to about 3000:1, about 2500:1 to about 3500:1, about 2500:1 to about 4000:1, about 2500:1 to about 4500:1, about 2500:1 to about 5000:1, about 3000:1 to about 3500:1, about 3000:1 to about 4000:1, about 3000:1 to about 4500:1, about 3000:1 to about 5000:1, about 3500:1 to about 4000:1, about 3500:1 to about 4500:1, about 3500:1 to about 5000:1, about 4000:1 to about 4500:1, or about 4000:1 to about 5000:1. In certain embodiments, the molar ratio of binding molecule: sialyltransferase can be about 200:1, 175:1, 150:1, 155:1, 140:1, 135:1, 130:1, 125:1, 120:1, 115:1, 110:1, 105:1, 100:1, 95:1, 90:1, 85:1, 80:1, 75:1, 70:1, 65:1, 60:1, 55:1, or 50:1. For example, the mass ratio of binding molecule: sialic acid substrate: sialyltransferase can be about 2000:500:1. In certain embodiments, the molar ratio of binding molecule: sialyltransferase can be about 200:1, 175:1, 150:1, 155:1, 140:1, 135:1, 130:1, 125:1, 120:1, 115:1, 110:1, 105:1, 100:1, 95:1, 90:1, 85:1, 80:1, 75:1, 70:1, 65:1, 60:1, 55:1, or 50:1. In certain embodiments the molar ratio of binding molecule to sialyltransferase can be about 80:1. As mentioned elsewhere herein, production of GEMs can further include contacting the monoclonal population of binding molecules with a galactosyltransferase, e.g., a soluble variant of human beta-1,4-galactosyltransferase 4 (B4GALT4) (SEQ ID NO: 4) and a galactose substrate, e.g., uridine-diphosphate-α-D-galactose (UDP-Gal). The contacting with galactosyltransferase and galactose substrate can occur prior to or simultaneously with the contacting with the soluble sialyltransferase and sialic acid substrate.

In some embodiments, the mass ratio of sialic acid substrate: sialyltransferase can be about 5:1 to about 3000:1, such as about 5:1 to about 10:1, about 5:1 to about 50:1, about 5:1 to about 100:1, about 5:1 to about 500:1, about 5:1 to about 1000:1, about 5:1 to about 1500:1, about 5:1 to about 2000:1, about 5:1 to about 2500:1, about 10:1 to about 50:1, about 10:1 to about 100:1, about 10:1 to about 500:1, about 10:1 to about 1000:1, about 10:1 to about 1500:1, about 10:1 to about 2000:1, about 10:1 to about 2500:1, about 10:1 to about 3000:1, about 50:1 to about 100:1, about 50:1 to about 500:1, about 50:1 to about 1000:1, about 50:1 to about 1500:1, about 50:1 to about 2000:1, about 50:1 to about 2500:1, about 50:1 to about 3000:1, about 100:1 to about 500:1, about 100:1 to about 1000:1, about 100:1 to about 1500:1, about 100:1 to about 2000:1, about 100:1 to about 2500:1, about 100:1 to about 3000:1, about 500:1 to about 1000:1, about 500:1 to about 1500:1, about 500:1 to about 2000:1, about 500:1 to about 2500:1, about 500:1 to about 3000:1, about 1000:1 to about 1500:1, about 1000:1 to about 2000:1, about 1000:1 to about 2500:1, about 1000:1 to about 3000:1, about 1500:1 to about 2000:1, about 1500:1 to about 2500:1, about 1500:1 to about 3000:1, about 2000:1 to about 2500:1, about 2000:1 to about 3000:1, or about 2500:1 to about 3000:1.

In some embodiments, the mass ratio of antibody: sialic acid substrate can be about 1:1 to about 40:1, such as about 1:1 to about 2:1, about 1:1 to about 4:1, about 1:1 to about 6:1, about 1:1 to about 8:1, about 1:1 to about 10:1, about 1:1 to about 15:1, about 1:1 to about 20:1, about 2:1 to about 4:1, about 2:1 to about 6:1, about 2:1 to about 8:1, about 2:1 to about 10:1, about 2:1 to about 15:1, about 2:1 to about 20:1, about 2:1 to about 40:1, about 4:1 to about 6:1, about 4:1 to about 8:1, about 4:1 to about 10:1, about 4:1 to about 15:1, about 4:1 to about 20:1, about 4:1 to about 40:1, about 6:1 to about 8:1, about 6:1 to about 10:1, about 6:1 to about 15:1, about 6:1 to about 20:1, about 6:1 to about 40:1, about 8:1 to about 10:1, about 8:1 to about 15:1, about 8:1 to about 20:1, about 8:1 to about 40:1, about 10:1 to about 15:1, about 10:1 to about 20:1, about 10:1 to about 40:1, about 15:1 to about 20:1, about 15:1 to about 40:1, or about 20:1 to about 40:1.

In some embodiments, the method comprises contacting the monoclonal population of binding molecules with a soluble sialyltransferase and a sialic acid substrate for at least 30 minutes, such as at least 45 minutes, at least 1 hour, at least 2 hours, at least 3 hours, at least 4 hours, at least 5 hours, at least 6 hours, at least 7 hours, at least 10 hours, at least 12 hours, at least 18 hours, at least 24 hours, at least 36 hours, or at least 48 hours. In some embodiments, the contacting occurs for about 30 minutes to about 48 hours, such as about 30 minutes to about 4 hours, about 30 minutes to about 5 hours, about 30 minutes to about 6 hours, about 30 minutes to about 7 hours, about 30 minutes to about 10 hours, about 30 minutes to about 12 hours, about 30 minutes to about 18 hours, about 30 minutes to about 24 hours, about 30 minutes to about 36 hours, about 2 hours to about 48 hours, about 3 hours to about 6 hours, about 3 hours to about 10 hours, about 3 hours to about 12 hours, about 3 hours to about 18 hours, about 3 hours to about 24 hours, about 3 hours to about 36 hours, about 3 hours to about 48 hours, about 4 hours to about 10 hours, about 4 hours to about 12 hours, about 4 hours to about 18 hours, about 4 hours to about 24 hours, about 4 hours to about 36 hours, about 4 hours to about 48 hours, about 5 hours to about 10 hours, about 5 hours to about 12 hours, about 5 hours to about 18 hours, about 5 hours to about 24 hours, about 5 hours to about 36 hours, about 5 hours to about 48 hours, about 7 hours to about 10 hours, about 7 hours to about 12 hours, about 7 hours to about 18 hours, about 7 hours to about 24 hours, about 7 hours to about 36 hours, about 7 hours to about 48 hours, about 10 hours to about 18 hours, about 10 hours to about 24 hours, about 10 hours to about 36 hours, about 10 hours to about 48 hours, about 12 to about 18 hours, about 12 hours to about 24 hours, about 12 hours to about 36 hours, about 12 hours to about 48 hours, about 18 hours to about 24 hours, about 18 hours to about 36 hours, about 18 hours to about 48 hours, about 24 hours to about 36 hours, about 24 hours to about 48 hours, or about 36 hours to about 48 hours,

In some embodiments, the method comprises contacting the monoclonal population of binding molecules with a soluble sialyltransferase and a sialic acid substrate at a temperature at about 2° C. to about 40° C., such as at about 2° C. to about 37° C., 2° C. to about 30° C., 2° C. to about 25° C., 2° C. to about 22° C., 2° C. to about 20° C., 2° C. to about 10° C., about 4° C. to about 40° C., about 4° C. to about 37° C., 4° C. to about 30° C., 4° C. to about 25° C., 4° C. to about 22° C., 4° C. to about 20° C., 4° C. to about 10° C., about 10° C. to about 40° C., about 10° C. to about 37° C., 10° C. to about 30° C., 10° C. to about 25° C., 10° C. to about 22° C., 10° C. to about 20° C., about 20° C. to about 40° C., about 20° C. to about 37° C., 20° C. to about 30° C., 20° C. to about 25° C., 20° C. to about 22° C., about 22° C. to about 40° C., about 22° C. to about 37° C., 22° C. to about 30° C., 22° C. to about 25° C., about 25° C. to about 40° C., about 25° C. to about 37° C., 25° C. to about 30° C., about 30° C. to about 40° C., or about 30° C. to about 37° C.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

### Exemplary Embodiments

Among the provided embodiments are:

Embodiment 1. A monoclonal population of multimeric binding molecules, each binding molecule comprising ten or twelve IgM-derived heavy chains, wherein the IgM-derived heavy chains comprise glycosylated IgM heavy chain constant regions each associated with a binding domain that specifically binds to a target, wherein each IgM heavy chain constant region comprises at least one, at least two, at least three, at least four, or at least five asparagine (N)-linked glycosylation motifs, wherein an N-linked glycosylation motif comprises the amino acid sequence N-X1-S/T, wherein N is asparagine, X1 is any amino acid except proline, and S/T is serine or threonine, wherein at least one, at least two, or at least three of the N-linked glycosylation motifs on each IgM heavy chain constant region are occupied by a complex glycan, and wherein the monoclonal population of binding molecules comprises at least thirty-five (35) moles sialic acid per mole of the binding molecules.

Embodiment 2. The monoclonal population of binding molecules of embodiment 1, comprising at least 40, at least 45, at least 50, at least 55, at least 60, or at least 65 moles sialic acid per mole of binding molecule.

Embodiment 3. The monoclonal population of binding molecules of embodiment 1, comprising about 40 to about 70, about 40 to about 60, about 40 to about 55, about 40 to about 50, about 50 to about 70, about 60 to about 70 moles sialic acid per mole of binding molecule.

Embodiment 4. The monoclonal population of binding molecules of any one of embodiments 1 to 3, wherein the IgM heavy chain constant regions are human IgM heavy chain constant regions or variants thereof comprising five N-linked glycosylation motifs N-X1-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04).

Embodiment 5. The monoclonal population of binding molecules of embodiment 4, wherein motifs N1, N2, and N3 are occupied by complex glycans.

Embodiment 6. The monoclonal population of binding molecules of any one of embodiments 1 to 5, produced by the method of cell line modification, in vitro glycoengineering, or any combination thereof.

Embodiment 7. The monoclonal population of binding molecules of embodiment 6, wherein the cell line modification comprises transfecting a cell line that produces the monoclonal population of binding molecules with a gene encoding a sialyltransferase, thereby producing a modified cell line that overexpresses the sialyltransferase.

Embodiment 8. The monoclonal population of binding molecules of embodiment 7, wherein the sialyltransferase comprises human beta-galactoside alpha-2,6-sialyltransferase 1 (ST6GAL1) (SEQ ID NO: 3).

Embodiment 9. The monoclonal population of binding molecules of embodiment 7 or embodiment 8, wherein the cell line modification further comprises transfecting a cell line that produces the monoclonal population of binding molecules with a gene encoding a galactosyltransferase, thereby producing a modified cell line that overexpresses the galactosyltransferase.

Embodiment 10. The monoclonal population of binding molecules of embodiment 9, wherein the galactosyltransferase comprises human beta-1,4-galactosyltransferase 4 (B4GALT4) (SEQ ID NO: 4).

Embodiment 11. The monoclonal population of binding molecules of any one of embodiments 6 to 10, wherein in vitro glycoengineering comprises contacting the monoclonal population of binding molecules with a soluble sialyltransferase and a sialic acid substrate.

Embodiment 12. The monoclonal population of binding molecules of embodiment 11, wherein the sialyltransferase comprises a soluble variant of human beta-galactoside alpha-2,6-sialyltransferase 1 (ST6GAL1) (SEQ ID NO: 3).

Embodiment 13. The monoclonal population of binding molecules of embodiment 12, wherein the soluble variant of ST6GAL1 comprises amino acids x to 406 of SEQ ID NO: 3, wherein x is an integer from 27 to 120.

Embodiment 14. The monoclonal population of binding molecules of embodiment 13, wherein the soluble variant of ST6GAL1 comprises amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3.

Embodiment 15. The monoclonal population of binding molecules of any one of embodiments 11 to 14, wherein the sialic acid substrate comprises cytidine monophosphate-N-acetyl-neuraminic acid (CMP-NANA).

Embodiment 16. The monoclonal population of binding molecules of any one of embodiments 11 to 15, wherein the mass ratio of binding molecule: sialic acid substrate is about 1:4 to about 40:1.

Embodiment 17. The monoclonal population of binding molecules of any one of embodiments 11 to 16, wherein the mass ratio of binding molecule: sialyltransferase is about 80:1 to about 5000:1.

Embodiment 18. The monoclonal population of binding molecules of any one of embodiments 11 to 17, wherein the mass ratio of binding molecule: sialyltransferase is

Embodiment 19. The monoclonal population of binding molecules of embodiment 18, wherein the mass ratio of binding molecule: sialic acid substrate: sialyltransferase is about 2000:500:1.

Embodiment 20. The monoclonal population of binding molecules of any one of embodiments 11 to 17, wherein the molar ratio of binding molecule: sialyltransferase is about 80:1.

Embodiment 21. The monoclonal population of binding molecules of embodiment 20, wherein the molar ratio of binding molecule: sialic acid substrate: sialyltransferase is about 80:500:1.

Embodiment 22. The monoclonal population of binding molecules of any one of embodiments 11 to 17, wherein the mass ratio of binding molecule: sialyltransferase is about 500:1.

Embodiment 23. The monoclonal population of binding molecules of embodiment 22, wherein the mass ratio of binding molecule: sialic acid substrate: sialyltransferase is about 500:62.5:1.

Embodiment 24. The monoclonal population of binding molecules of any one of embodiments 11 to 23, wherein the contacting of the monoclonal population of binding molecules with the soluble sialyltransferase and the sialic acid substrate comprises at least 30 minutes of contact.

Embodiment 25. The monoclonal population of binding molecules of embodiment 24, wherein the contacting comprises at least 1 hour, 2 hours, 3 hours, 4 hours, 5 hours, 6 hours, 7 hours, 24 hours, 36 hours, or 48 hours of contact.

Embodiment 26. The monoclonal population of binding molecules of any one of embodiments 11 to 25, wherein the contacting of the monoclonal population of binding molecules with the soluble sialyltransferase and the sialic acid substrate occurs at about 2° C. to about 40° C.

Embodiment 27. The monoclonal population of binding molecules of embodiment 26, wherein the contacting occurs at 15° C. to about 37° C., 15° C. to about 30° C., or 15° C. to about 25° C.

Embodiment 28. The monoclonal population of binding molecules of any one of embodiments 11 to 27, wherein in vitro glycoengineering further comprises contacting the monoclonal population of binding molecules with a galactosyltransferase and a galactose substrate.

Embodiment 29. The monoclonal population of binding molecules of embodiment 28, wherein the galactosyltransferase comprises a soluble variant of human beta-1,4-galactosyltransferase 4 (B4GALT4) (SEQ ID NO: 4).

Embodiment 30. The monoclonal population of binding molecules of embodiment 29, wherein the soluble variant of B4GALT4 comprises amino acids x to 344 of SEQ ID NO: 4, wherein x is an integer from 39 to 120.

Embodiment 31. The monoclonal population of binding molecules of embodiment 30, wherein the soluble variant of B4GALT4 comprises amino acids 120 to 344, 115 to 344, 110 to 344, 105 to 344, 100 to 344, 95 to 344, 90 to 344, 85 to 344, 80 to 344, 75 to 344, 70 to 344, 65 to 344, 60 to 344, 55 to 344, 50 to 344, 45 to 344, 40 to 344, or 39 to 344 of SEQ ID NO: 4.

Embodiment 32. The monoclonal population of binding molecules of any one of embodiments 28 to 31, wherein the galactose substrate comprises uridine-diphosphate-α-D-galactose (UDP-Gal).

Embodiment 33. The monoclonal population of binding molecules of any one of embodiments 28 to 32, wherein the contacting with the galactosyltransferase and the galactose substrate occurs prior to or simultaneously with the contacting with the soluble sialyltransferase and sialic acid substrate.

Embodiment 34. The monoclonal population of binding molecules of any one of embodiments 1 to 33, wherein each binding molecule is multispecific, and wherein two or more binding domains associated with the IgM heavy chain constant regions of each binding molecule specifically bind to different targets.

Embodiment 35. The monoclonal population of binding molecules of any one of embodiments 1 to 33, wherein the binding domains associated with the IgM heavy chain constant regions of each binding molecule specifically bind to the same target.

Embodiment 36. The monoclonal population of binding molecules of embodiment 35, wherein the binding domains associated with the IgM heavy chain constant regions of each binding molecule are identical.

Embodiment 37. The monoclonal population of binding molecules of any one of embodiments 34 to 36, wherein the binding domains are antibody-derived antigen-binding domains.

Embodiment 38. The monoclonal population of binding molecules of embodiment 37, wherein each binding molecule is a pentameric or a hexameric IgM antibody comprising five or six bivalent IgM binding units, respectively, wherein each binding unit comprises two IgM heavy chains each comprising a VH situated amino terminal to the variant IgM constant region, and two immunoglobulin light chains each comprising a light chain variable domain (VL) situated amino terminal to an immunoglobulin light chain constant region, and wherein the VH and VL combine to form an antigen-binding domain that specifically binds to the target.

Embodiment 39. The monoclonal population of binding molecules of embodiment 38, wherein each antigen-binding domain of each binding molecule binds to the same target.

Embodiment 40. The monoclonal population of binding molecules of embodiment 39, wherein each antigen-binding domain of each binding molecule is identical.

Embodiment 41. The monoclonal population of binding molecules of any one of embodiments 1 to 40, wherein the target is a target epitope, a target antigen, a target cell, a target organ, or a target virus.

Embodiment 42. The monoclonal population of binding molecules of any one of embodiments 1 to 41, wherein each binding molecule is pentameric and further comprises a J-chain, or functional fragment thereof, or a functional variant thereof.

Embodiment 43. The monoclonal population of binding molecules of embodiment 42, wherein the J-chain is a mature human J-chain comprising the amino acid sequence SEQ ID NO: 6 or a functional fragment thereof, or a functional variant thereof.

Embodiment 44. The monoclonal population of binding molecules of embodiment 43, wherein the J-chain comprises an N-linked glycosylation motif N-X1-S/T starting at amino acid positions corresponding to amino acid 49 of SEQ ID NO: 6 (motif N6).

Embodiment 45. The monoclonal population of binding molecules of any one of embodiments 42 to 44, wherein the J-chain is a functional variant J-chain comprising one or more single amino acid substitutions, deletions, or insertions relative to a reference J-chain identical to the variant J-chain except for the one or more single amino acid substitutions, deletions, or insertions, and wherein the monoclonal population of binding molecules exhibits an increased serum half-life upon administration to a subject animal relative to a reference IgM-derived binding molecule that is identical except for the one or more single amino acid substitutions, deletions, or insertions in the variant J-chain, and is administered using the same method to the same animal species.

Embodiment 46. The monoclonal population of binding molecules of embodiment 45, wherein the variant J-chain or functional fragment thereof comprises one, two, three, or four single amino acid substitutions, deletions, or insertions relative to the reference J-chain.

Embodiment 47. The monoclonal population of binding molecules of embodiment 45 or embodiment 46, wherein the variant J-chain or functional fragment thereof comprises an amino acid substitution at the amino acid position corresponding to amino acid Y102 of the wild-type mature human J-chain of SEQ ID NO: 6.

Embodiment 48. The monoclonal population of binding molecules of embodiment 47, wherein the amino acid corresponding to Y102 of SEQ ID NO: 6 is substituted with alanine (A).

Embodiment 49. The monoclonal population of binding molecules of embodiment 48, wherein the J-chain comprises the amino acid sequence SEQ ID NO: 7.

Embodiment 50. The monoclonal population of binding molecules of any one of embodiments 42 to 49, wherein the J-chain or fragment or variant thereof is a modified J-chain further comprising a heterologous moiety, wherein the heterologous moiety is fused or conjugated to the J-chain or fragment or variant thereof.

Embodiment 51. The monoclonal population of binding molecules of embodiment 50, wherein the heterologous moiety is a polypeptide fused to the J-chain or fragment or variant thereof.

Embodiment 52. The monoclonal population of binding molecules of embodiment 51, wherein the heterologous polypeptide is fused to the J-chain or fragment or variant thereof via a peptide linker.

Embodiment 53. The monoclonal population of binding molecules of embodiment 52, wherein the peptide linker comprises at least 5 amino acids, but no more than 25 amino acids.

Embodiment 54. The monoclonal population of binding molecules of embodiment 52 or embodiment 53, wherein the peptide linker consists of GGGGSGGGGSGGGGS (SEQ ID NO: 43).

Embodiment 55. The monoclonal population of binding molecules of any one of embodiments 51 to 54, wherein the heterologous polypeptide is fused to the N-terminus of the J-chain or fragment or variant thereof or to the C-terminus of the J-chain or fragment or variant thereof.

Embodiment 56. The monoclonal population of binding molecules of any one of embodiments 51 to 55, wherein heterologous moieties that can be the same or different are fused to the N-terminus and C-terminus of the J-chain or fragment or variant thereof.

Embodiment 57. The monoclonal population of binding molecules of any one of embodiments 51 to 56, wherein the heterologous polypeptide comprises a binding domain.

Embodiment 58. The monoclonal population of binding molecules of embodiment 57, wherein the binding domain of the heterologous polypeptide is an antibody or antigen-binding fragment thereof.

Embodiment 59. The monoclonal population of binding molecules of embodiment 58, wherein the antigen-binding fragment is a scFv fragment.

Embodiment 60. The monoclonal population of binding molecules of embodiment 59, wherein the heterologous scFv fragment binds to CD3c.

Embodiment 61. The monoclonal population of binding molecules of embodiment 60, wherein the modified J-chain comprises the amino acid sequence SEQ ID NO: 36 (V15J), SEQ ID NO: 37 (V15J*), SEQ ID NO: 38 (SP), SEQ ID NO: 31 (A-55-J*), SEQ ID NO: 32 (A-56-J*), SEQ ID NO: 33 (A-57-J*), amino acids 20-420 of SEQ ID NO: 34 (VJH), amino acids 20-420 of SEQ ID NO: 35 (VPH), or SEQ ID NOs: 6 or 7 fused via a peptide linker to an anti-CD3c scFv comprising HCDR1, HCDR2, HCDR3, LCDR1, LCDR2, and LCDR3 amino acid sequences comprising SEQ ID NO: 15, SEQ ID NO: 16, SEQ ID NO: 17, SEQ ID NO: 19, SEQ ID NO: 20, and SEQ ID NO: 21, respectively.

Embodiment 62. A pharmaceutical composition comprising the monoclonal population of binding molecules of any one of embodiments 1 to 61 and a pharmaceutically acceptable excipient.

Embodiment 63. A recombinant host cell that produces the monoclonal population of binding molecules of any one of embodiments 1 to 61.

Embodiment 64. A method of producing the monoclonal population of binding molecules of any one of embodiments 1 to 61, comprising culturing the host cell of embodiment 62, and recovering the population of binding molecules.

Embodiment 65. A method for producing a monoclonal population of highly sialylated multimeric binding molecules, comprising providing a cell line that expresses the monoclonal population of binding molecules, culturing the cell line, and recovering the monoclonal population of binding molecules, wherein each binding molecule comprises ten or twelve IgM-derived heavy chains, wherein the IgM-derived heavy chains comprise glycosylated IgM heavy chain constant regions each associated with a binding domain that specifically binds to a target, wherein each IgM heavy chain constant region comprises at least three, at least four, or at least five asparagine(N)-linked glycosylation motifs, wherein an N-linked glycosylation motif comprises the amino acid sequence N-X1-S/T, wherein N is asparagine, X1 is any amino acid except proline, and S/T is serine or threonine, wherein on average at least one, at least two, or at least three of the N-linked glycosylation motifs on each IgM heavy chain constant region in the population are occupied by complex glycans, and wherein the cell line, recovery process, or a combination thereof is optimized to enrich for complex glycans comprising at least one, two, three, or four sialic acid terminal monosaccharides per glycan.

Embodiment 66. The method of embodiment 65, wherein the cell line, recovery process, or a combination thereof is optimized to result in a monoclonal population of binding molecules comprising at least 30, at least 35, at least 40, at least 45, at least 50, at least 60, or at least 65 moles sialic acid per mole of binding molecule.

Embodiment 67. The method of embodiment 66, wherein the cell line, recovery process, or a combination thereof is optimized to result in a monoclonal population of binding molecules comprising about 40 to about 70, about 40 to about 60, about 40 to about 55, about 40 to about 50, about 50 to about 70, about 60 to about 70 moles sialic acid per mole of binding molecule.

Embodiment 68. The method of any one of embodiments 65 to 67, wherein the IgM heavy chain constant regions are derived from human IgM heavy chain constant regions comprising five N-linked glycosylation motifs N-X1-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04).

Embodiment 69. The method of embodiment 68, wherein on average one, two, or all three of motifs N1, N2, and N3 in the population of binding molecules are occupied by complex glycans.

Embodiment 70. The method of any one of embodiments 65 to 69, wherein the provided cell line is modified to overexpress a sialyltransferase.

Embodiment 71. The method of embodiment 70, wherein the sialyltransferase comprises human beta-galactoside alpha-2,6-sialyltransferase 1 (ST6GAL1, SEQ ID NO: 3).

Embodiment 72. The method of any one of embodiments 65 to 71, wherein the recovery process comprises subjecting the monoclonal population of binding molecules to in vitro glycoengineering.

Embodiment 73. The method of embodiment 72, wherein the in vitro glycoengineering comprises contacting the monoclonal population of binding molecules with a soluble sialyltransferase and a sialic acid substrate.

Embodiment 74. The method of embodiment 73, wherein the sialyltransferase comprises a soluble variant of human beta-galactoside alpha-2,6-sialyltransferase 1 (ST6GAL1) (SEQ ID NO: 3).

Embodiment 75. The method of embodiment 74, wherein the soluble variant of ST6GAL1 comprises amino acids x to 406 of SEQ ID NO: 3, wherein x is an integer from 27 to 120.

Embodiment 76. The method of embodiment 75, wherein the soluble variant of ST6GAL1 comprises amino acids 120 to 406, 115 to 406, 110 to 406, 109 to 406, 105 to 406, 100 to 406, 95 to 406, 90 to 406, 89 to 406, 88 to 406, 87 to 406, 86 to 406, 85 to 406, 84 to 406, 83 to 406, 82 to 406, 81 to 406, 80 to 406, 75 to 406, 70 to 406, 65 to 406, 60 to 406, 55 to 406, 50 to 406, 45 to 406, 40 to 406, 35 to 406, 30 to 406, or 27 to 406 of SEQ ID NO: 3.

Embodiment 77. The method of any one of embodiments 73 to 75, wherein the sialic acid substrate comprises cytidine monophosphate (CMP)-N-acetyl-neuraminic acid (CMP-NANA).

Embodiment 78. The method of any one of embodiments 73 to 77, wherein the mass ratio of binding molecule: sialic acid substrate is about 1:4 to about 40:1.

Embodiment 79. The method of any one of embodiments 73 to 78, wherein the mass ratio of binding molecule: sialyltransferase is about 80:1 to about 10000:1.

Embodiment 80. The method of any one of embodiments 73 to 79, wherein the mass ratio of binding molecule: sialyltransferase is about 2000:1.

Embodiment 81. The method of embodiment 80, wherein the mass ratio of binding molecule: sialic acid substrate: sialyltransferase is about 2000:500:1.

Embodiment 82. The method of any one of embodiments 73 to 79, wherein the molar ratio of binding molecule: sialyltransferase is about 80:1.

Embodiment 83. The method of embodiment 82, wherein the molar ratio of binding molecule: sialic acid substrate: sialyltransferase is about 80:500:1.

Embodiment 84. The method of any one of embodiments 73 to 79, wherein the mass ratio of binding molecule: sialyltransferase is about 500:1.

Embodiment 85. The method of embodiment 84, wherein the mass ratio of binding molecule: sialic acid substrate: sialyltransferase is about 500:62.5:1.

Embodiment 86. The method of any one of embodiments 73 to 85, wherein the contacting of the monoclonal population of binding molecules with the soluble sialyltransferase and the sialic acid substrate comprises at least 30 minutes of contact.

Embodiment 87. The method of embodiment 86, wherein the contacting comprises at least 1 hour, 2 hours, 3 hours, 4 hours, 5 hours, 6 hours, 7 hours, 24 hours, 36 hours, or 48 hours of contact.

Embodiment 88. The method of any one of embodiments 73 to 87, wherein the contacting of the monoclonal population of binding molecules with the soluble sialyltransferase and the sialic acid substrate occurs at about 2° C. to about 40° C.

Embodiment 89. The method of embodiment 88, wherein the contacting occurs at 15° C. to about 37° C., 15° C. to about 30° C., or 15° C. to about 25° C.

Embodiment 90. The method of any one of embodiments 73 to 77, wherein in vitro glycoengineering further comprises contacting the monoclonal population of binding molecules with a galactosyltransferase and a galactose substrate.

Embodiment 91. The method of embodiment 90, wherein the galactosyltransferase comprises a soluble variant of human beta-1,4-galactosyltransferase 4 (B4GALT4) (SEQ ID NO: 4).

Embodiment 92. The method of embodiment 90 or embodiment 91, wherein the galactose substrate comprises uridine-diphosphate-α-D-galactose (UDP-Gal).

Embodiment 93. The method of any one of embodiments 90 to 92, wherein the contacting with the galactosyltransferase and a galactose substrate occurs prior to or simultaneously with the contacting with the soluble sialyltransferase and a sialic acid substrate.

The following examples are offered by way of illustration and not by way of limitation.

### EXAMPLES

### Example 1

### Materials and Methods

Population of IgM Antibodies

Except as noted below, these experiments were carried out on a monoclonal population of the IgM bispecific antibody CD20×CD3 IGM-A, which includes IgM heavy chains comprising wild-type human IgM constant regions (e.g., SEQ ID NO: 1 or SEQ ID NO: 2) and an anti-CD20 VH region of SEQ ID NO: 8, light chains comprising the antiCD20 VL region of SEQ ID NO: 9, and a modified J-chain that binds to CD3 comprising amino acids 20-420 of SEQ ID NO: 34. CD20×CD3 IGM-A is described in detail in U.S. Patent Application Publication No. US-2018-0265596-A1, which is incorporated herein by reference in its entirety. Glycoengineered IgM antibodies, IgM-like antibodies, or IgM-derived binding molecules are referred to as “GEMs” throughout the Examples regardless of the method of glycoengineering.

Glycoengineering of IgM Antibody Populations

Along with various controls, varying amounts of a truncated version of human α-2,6 sialyltransferase as indicated in the Examples below (“truncated human ST6,” available from Roche Diagnostics, Inc. (Material Number 07012250103 or Material Number 08098174103)) or from Agilent (Part Number GKT-S26) was added to a 20 μl reaction solution that contained a partially purified monoclonal population of an IgM antibody, e.g., anti-CD20×CD3 IGM-A, and varying amounts of cytidine-5′-monophospho-N-acetylneuraminic acid sodium salt (CMP-NANA), dissolved in 50 mM Tris-acetate (pH 7.5). The reactions were allowed to run for 8 hours at 37° C. unless indicated otherwise. Reactions were stopped by freezing at −20° C. The ST6 treated IgM populations were further purified, e.g., by anion exchange chromatography and/or mixed mode chromatography, prior to further analyses.

Total Sialic Acid Quantitation

The Sialic Acid (NANA) Assay Kit (Agilent AdvanceBio Total Sialic Acid Quantitation Kit) measures free or released sialic acid (N-acetylneuraminic acid (NANA)) from glycoproteins. The assay uses an enzyme coupled reaction in which oxidation of free sialic acid creates an intermediate that reacts stoichiometrically with the probe to generate a product that can be detected by absorbance (OD=530 nm) or fluorescence (excitation/emission (Ex/Em)=530/590 nm). The kit measures sialic acid in the linear range of 40 pmol-1,000 pmol with a detection sensitivity of 0.15 mg/ml concentration for IgM antibodies. The kit was used according to manufacturers' recommendations. Briefly, samples were digested with sialidase A for 2 hours. A Bovine Fetuin Control protein was used as a positive control with an expected range of 9.6-13.9 mol/mol. Sialic acid standards were prepared with the following pmol for fluorescent measurement: 1,000, 500, 250 and 0 pmol. Conversion and Developer mix was then prepared according to the Table 2 below.

TABLE 2
Sialic Acid Quantitation Assay

| | Conversion | | | | | |
|---|---|---|---|---|---|---|
| | Reagent | HRP | SAQ | | | |
| Number of | Solution | Solution | Buffer C | SAQ Dye | Total | Number of |
| Wells | (μL) | (μL) | (μL) | (μL) | (μL) | Wells |
| n | 30/sample | 15/sample | 5/sample | 5/sample | 55 | N |
| 16 | 480 | 240 | 80 | 80 | 880 | 16 |
| 36 | 1080 | 540 | 180 | 180 | 1980 | 36 |
| 48 | 1440 | 720 | 240 | 240 | 2640 | 48 |

Once sialic acid is released by the sialidase A digestion, N-acetylneuraminic aldolase catalyzes the reaction to form pyruvic acid. The reaction then goes through an additional step with pyruvate oxidase as the catalyst to form hydrogen peroxide which forms a 1:1 complex with the dye to form a fluorescence reporter dye. This dye can be read by fluorescence detection (Ex/Em=530/590 nm) which is then correlated to give a sialic acid level mol/mol from the sialic acid standard curve.

### Example 2

### Effect of α-2,6 Sialyltransferase Concentration on Sialylation of IgM Antibody Populations

Varying amounts of truncated human ST6 were added to a 20 μl reaction solution comprising 60 μg of anti-CD20×CD3 IGM-A (3 mg/ml) and 30 μg of cytidine-5′-monophospho-N-acetylneuraminic acid sodium salt (CMP-NANA, 1.5 mg/ml) as described in Example 1. The resulting sialylation was quantitated as described in Example 1, and the amounts of sialylation compared to truncated human ST6 concentration are shown in FIG. 4.

These results demonstrate that ST6 concentrations as low as 1.5 μg/ml (molar ratio of IgM to truncated human ST6 of 80:1) can be used to generate SA level of 40 mol/mol, and as the truncated human ST6 concentration is increased to 30 μg/ml or higher (molar ratio of IgM to truncated human ST6 of about 4:1 or higher), SA levels greater than 60 mol/mol can be generated.

### Example 3

### Sialylation of Other IgM Antibodies

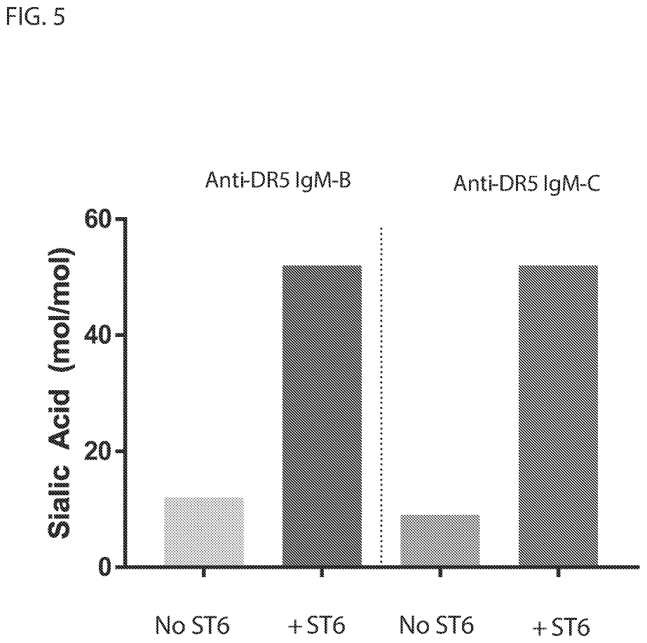

To determine if the in vitro sialylation procedures developed above could be applied to other IgM antibodies, two other CHO cell lines expressing recombinant IgM antibodies, pentameric anti-DRS IGM-B (VH: SEQ ID NO: 10, VL: SEQ ID NO: 11, see U.S. Pat. No. 7,521,048) and hexameric anti-DRS IGM-C (VH: SEQ ID NO: 12, VL: SEQ ID NO: 13, see U.S. Pat. No. 7,790,165), were sialylated and analyzed as described Example 1. The 20 μl reactions included 0.28 μg truncated human ST6 (final concentration of 14 μg/mL), 60 μg anti-DRS IGM-B or anti-DRS IGM-C (for an approximately 8:1 IgM to truncated human ST6 molar ratio), and 30 μg CMP-NANA were used. Control reactions without the truncated human ST6 were also run. The resulting sialylation was determined as described in Example 1 and the amounts of sialylation for each condition are shown in FIG. 5 and Table 3.

TABLE 3
Sialylation of Anti-DR5 Antibodies

| Antibody | Condition | SA (mol/mol) |
|---|---|---|
| Anti-DR5 IGM-B | No ST6 | 16 |
| Anti-DR5 IGM-B-GEM | +ST6 | 52 |
| Anti-DR5 IGM-C | No ST6 | 9 |
| Anti-DR5 IGM-C-GEM | +ST6 | 52 |

### Example 4

### Sialylation Level of Human Serum IgM

The sialylation level of human serum IgM (obtained from Sigma, cat #I8260-25 mg) was determined using the Sialic Acid (NANA) Assay Kit (Agilent AdvanceBio Total Sialic Acid Quantitation Kit) as explained in Example 1.

Once sialic acid is released by the sialidase A digest, N-acetylneuraminic aldolase catalyzes the reaction to form pyruvic acid. The reaction then goes through an additional step with pyruvate oxidase as the catalyst forming hydrogen peroxide which forms a 1:1 complex with the dye to form a fluorescence reporter dye. This dye can be read by fluorescence detection (Ex/Em=530/590 nm) which is then correlated to give a Sialic Acid level mol/mol from the sialic acid standard curve. The resulting amount of sialylation is shown in Table 4.

TABLE 4
Sialic Acid Content of Human Serum IgM

| Antibody | SA (mol/mol) |
|---|---|
| Human serum IgM | 30 |

### Example 5

### Effect of Increased Sialylation on IGM Antibodies

The glycoengineered IgM CD20×CD3 IGM-A (“anti-CD20×CD3 IGM-A-GEM”) material used in the experiments in this example had about 37 moles sialic acid/mole of IgM and was prepared as described in Example 1. The non-glycoengineered IGM CD20 x CD3 IGM-A had about 14 moles sialic acid/mole of IgM.

Complement Dependent Cytotoxicity

The CD20-expressing Ramos (ATCC cat. #CRL-1596) cells were cultured in RPMI (Invitrogen) supplemented with 10% Heat inactivated Fetal Bovine Serum (Gibco Cat. #16140-071). Ramos cells (50,000) were seeded in a 96-well plate in 10 ul/well volume. Cells were treated with 10 ul/well serial dilutions of 2 different lots of anti-CD20×CD3 IGM, or anti-CD20×CD3 IGM-A sialylated in vitro as described in Example 1 (“anti-CD20×CD3 IGM-A-GEM”). All the antibody dilutions were carried out in RPMI media supplemented with 10% heat inactivated serum. Human serum complement (Quidel cat. #A113) was added to the antibody-treated cells at a final concentration of 5% in 10 ul/well volume. The reaction mixtures were incubated at 37° C. for 4 hours. CELLTITER-GLO® reagent (Promega cat. #G7572) was added at a volume equal to the volume of culture medium present in each well. The plate was shaken for 2 minutes, incubated for 10 minutes at room temperature, and luminescence was measured on an Envision multimode reader (Perkin Elmer) using 0.1 s integration time per well. Data were analyzed using GraphPad Prism and a four-parameter fit with top and bottom values fixed at 100 and 0% viability, respectively. The concentration of antibody that yields a half-maximal response (EC₅₀) was calculated for each condition and is shown in Table 5. In vitro sialylation had no appreciable effect on complement-dependent cytotoxicity.

TABLE 5
CDC Activity

| Sample | EC₅₀ (pM) |
|---|---|
| anti-CD20 × CD3 IGM-A | 260 |
| anti-CD20 × CD3 IGM -A-GEM | 230 |

T-Cell Activation

T cell activation (TCA) by anti-CD20×CD3 IGM-A-GEM or anti-CD20×CD3 IGM-A was determined using a luminescence-based readout in the presence of antigen-positive Jurkat based reporter cells. Engineered Jurkat T-cells (Promega J1601 Part #J131A) and Ramos cells were cultured in RPMI (Invitrogen) supplemented with 10% Heat inactivated Fetal Bovine Serum (Gibco Cat. #16140-071). Ramos cells (7500 cells/well in 10 ul volume) were added in a white 384 well assay plate. Next, serial dilutions of anti-CD20×CD3 IGM-A-GEM or anti-CD20×CD3 IGM-A were added to Ramos cells in 10 μl volume. The engineered Jurkat cells (25000 cells/well in 20 μl volume) were added to mixture to final volume of 40 μL. The mixture was incubated for 16 h at 37° C. with 5% CO₂. The cell mixtures were then mixed with 20 μL lysis buffer containing luciferin (Promega, CELLTITER-GLO®) to measure luciferase reporter activity. Light output was measured by EnVision plate reader. EC₅₀ was determined by 4 parameter curve fit using Prism software.

The EC₅₀ was calculated for each condition and is shown in Table 6. In vitro sialylation had no appreciable effect on T cell activation.

TABLE 6
T cell Activation

| Sample | EC₅₀ (pM) |
|---|---|
| anti-CD20 × CD3 IGM-A | 29 |
| anti-CD20 × CD3 IGM-A-GEM | 22 |

B-Cell Killing In Vitro

Ramos, a CD19+CD20+ B cell line, was labeled with a cell tracing dye (Oregon

Green 488, ThermoFisher, Cat #C34555), and then co-cultured with primary human CD8+ T cells (Precision for Medicine, Cat #84300; Negatively selected) with serial dilutions of anti-CD20×CD3 IGM-A or anti-CD20×CD3 IGM-A-GEM for 48 hours at 37° C., 5% CO₂. Cells were harvested and stained with 7-AAD (BD Biosciences, Cat #559925) and analyzed by flow cytometry to assess viable B cells. The EC₅₀ was calculated for each condition and is shown in Table 7. In vitro sialylation had no appreciable effect on the ability of the antibody to kill B cells.

TABLE 7
T-Cell dependent B Cell Killing

| Sample | Max (%) | EC₅₀ (pM) |
|---|---|---|
| anti-CD20 × CD3 IGM-A | 94.5 | 6.57 |
| anti-CD20 × CD3 IGM-A-GEM | 94.8 | 7.99 |

Pharmacokinetics

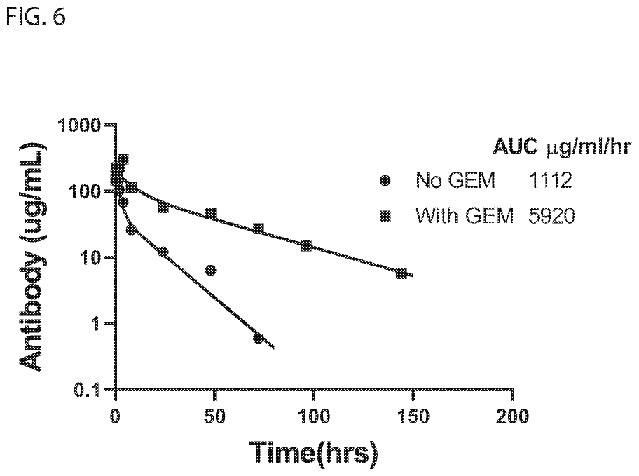

Pharmacokinetic parameters were measured for various IgM antibodies in an in vivo mouse model as follows. Balb/c mice were injected with 5 mg/kg of either anti-CD20×CD3 IGM-A or anti-CD20×CD3 IGM-A-GEM antibody via intravenous infusion. Blood samples were collected at 10 or 12 time points total for each antibody, with 2 mice per time point. Each mouse was bled once through the facial vein (100 μL) and then another time by terminal cardiac puncture (max obtainable, ˜500 μL). A standard ELISA assay was used to measure the serum concentration of each antibody in the blood at each time point. Quality metrics were verified on all ELISAs, and PK parameters, including T_{1/2-alpha}, T_{1/2-beta}, and the area under the concentration curve from time zero to infinity (AUC_{0-∞}, measured in μg/ml*hr) were derived using standard curve fitting techniques (Win Non Lin, Phoenix Software). The PK results, including area under the curve (AUC) are presented in FIG. 6.

### Example 6

### Cell Line Engineering to Increase Sialylation

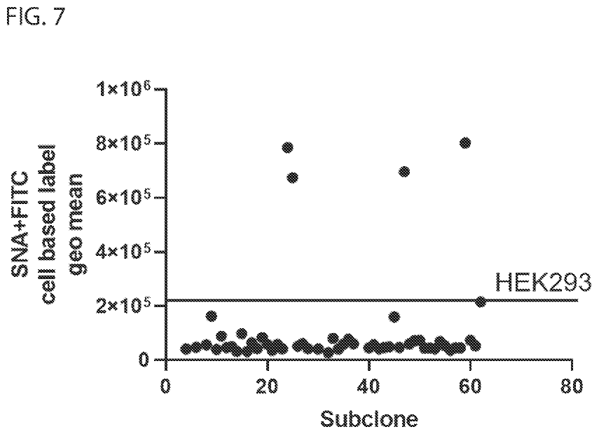

A vector for comprising a GACACC Kozak sequence, a sequence encoding an alpha-2,6-sialyltransferase (ST6) SEQ ID 3 (NCBI Reference Sequence: sp|P15907.1), and a hygromycin marker selection was generated by standard methods. The vector was electroporated into a stable CHO subclone expressing anti-CD20×CD3 IGM-A. After selection and recovery, the resulting pool was subcloned by limiting dilution into 384-well plates. Phenotypic screening was done by labelling the subcloned cells with SNA-1 conjugated to fluorescein isothiocyanate (FITC). SNA-1 is a lectin specific to 2,6-sialic acid. The cells themselves were labeled directly after being washed in FACS buffer. The fluorescence levels were detected, and the results are shown in FIG. 7. Only 4 of 60 subclones generated a signal above that of HEK293 cells, which were used as a positive control. Two subclones 25 and 47 were picked for further studies.

To detect the presence of ST6 in the genome of the subclones 25 and 47, QPCR analysis was done using the primers in Table 8.

TABLE 8
Primers used in a QPCR assay.

| | |
|---|---|
| Primer 1 | GAC CGA CGT GTG CTA CTA TTA C |
| | (SEQ ID NO: 39) |
| Primer 2 | GAG GTG CTT CAC GAG ATT CTT |
| | (SEQ ID NO 40) |

These primers are represented in the coding sequence (CDS) of the ST6 gene. Both subclones generated a positive response by the 38^{th} cycle, whereas a CHO cell control did not.

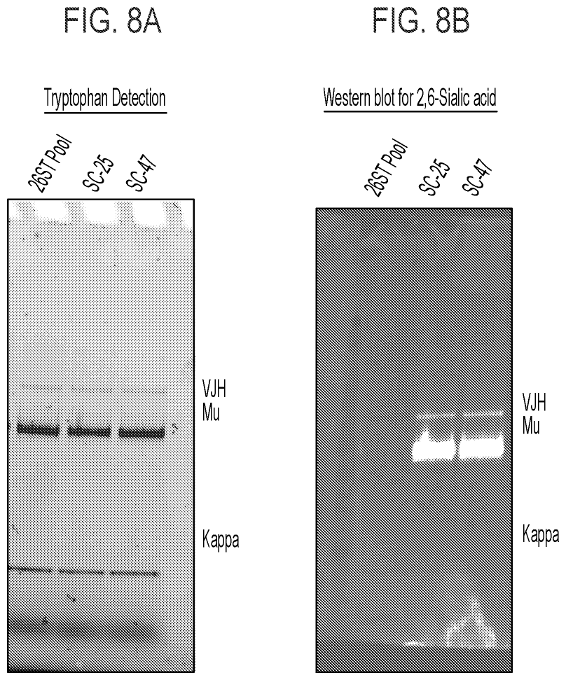

A western blot was performed to detect 2,6 linked sialic acid in the anti-CD20×CD3 IGM-A antibodies expressed and purified from small scale fermentations. The reduced, denatured gel (BioRad® CriterionTGX Stain-Free Precast gel) was visualized and imaged according to manufacturer's instructions. The resulting image is shown in FIG. 8A. The gel was then treated with a biotinylated SNA-I lectin and a streptavidin horseradish peroxidase fusion protein and was imaged. The resulting image is shown in FIG. 8B.

The selected subclones had detectable levels of 2,6-sialic acid; whereas the stable pool of CHO cells from which the subclones originated did not.

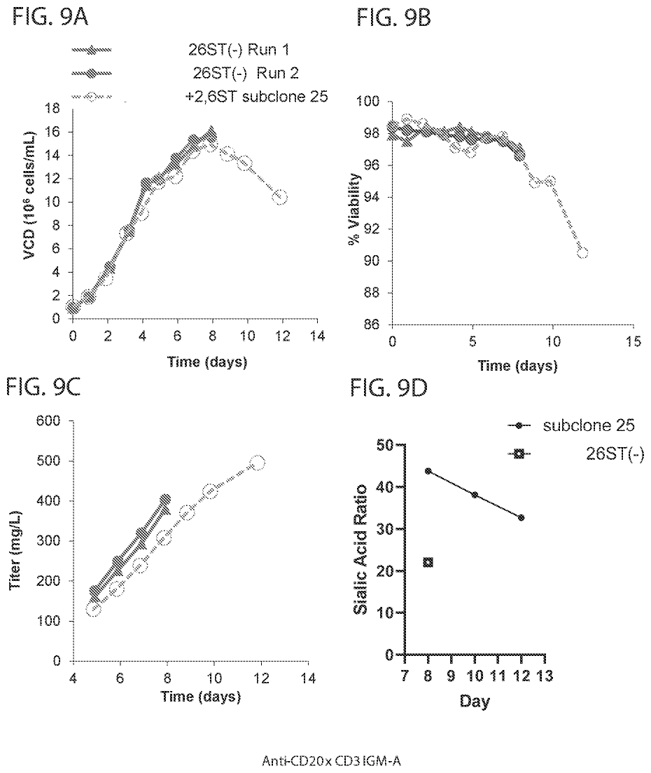

Subclone 25 was expanded for a 3-liter bioreactor production run so that a comparison study could be made against the parental anti-CD20×CD3 IGM-A produced in cells which did not have the 2,6-sialyltransferase gene. FIGS. 9A-D shows how the cultures performed in terms of viable cell density (FIG. 9A), cell-viability (FIG. 9B), production of anti-CD20×CD3 IGM-A (FIG. 9C), and the moles of sialic acid per mole of anti-CD20×CD3 IGM-A (FIG. 9D). Subclone 25 produced 25% less anti-CD20×CD3 IGM-A by day 8 than the parental cell line (FIG. 9C), but the sialic acid content was more than doubled and remained elevated (FIG. 9D). Day 12 data shows that subclone 25 produced 500 μg/ml (FIG. 9C) with a sialic acid content of 37 mol/mol of IgM (FIG. 9D).

### Example 7

### 2,6-Sialic Acid Knock-In Parental Cell Line

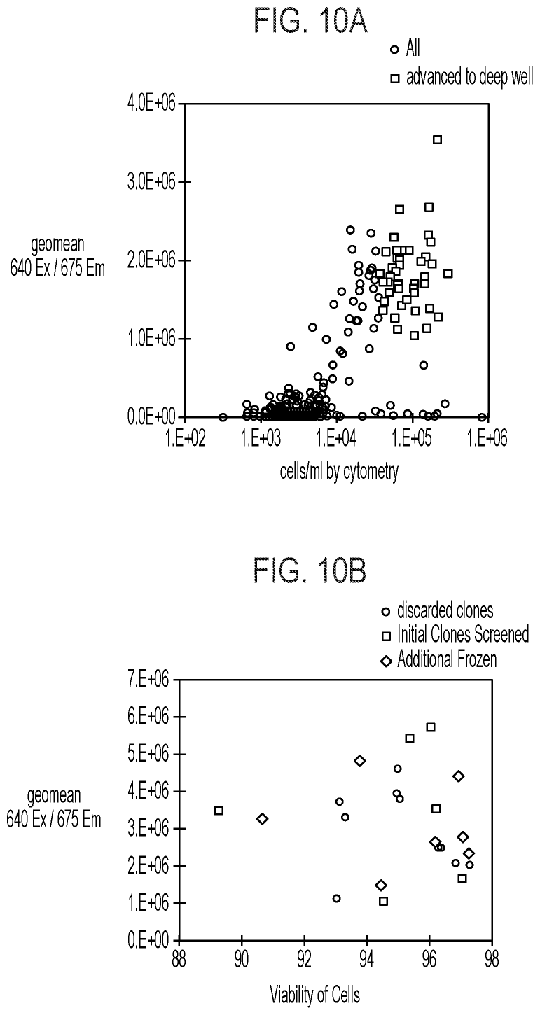

A vector for stable insertion of alpha-2,6-sialyltransferase (NCBI Reference Sequence: NP_775324.1) and a hygromycin marker selection was generated by a commercial vendor. The vector was electroporated into a CHO suspension cell-line. The resulting stable pool was cloned, and 384 clones were expanded and screened by cytometry. Cell surface-based labeling was done with a 2,6-sialic acid specific lectin (SNA-1) which was chemically conjugated to a Cy5 dye, the results of this assay are shown in FIG. 10A. Based on the screen 48 clones which were growing-out well and had high lectin labelling levels were expanded to 24-deep well plates and shaken at 300 RPM in an incubator at 37° C., 5% CO₂, and 80% humidity. An identical 2,6-sialic acid screen was done after the 48 clones had expanded in the deep well plate and the top 22 were transferred to shake flask.

The selected 22 clones were screened again when the cell densities were between 1-4 million cells/ml. Lectin labeling levels in this case are shown in FIG. 10B with the corresponding viability of the culture at the time they were assayed.

Based on the shake flask analysis six clones were chosen to be evaluated initially by transfection with two to four control IgMs. The parental CHO cell-line was also transfected with the same four control IgMs and used as a basis for comparison. After transfection four of the six selected clones were either not able to recover or had growth problems after or prior to transfection. Two clones (2B4 and 2C2) which were able to survive after transfection, showed higher titer in all but one case, and, in all cases, had higher sialic acid content than IgM's from the parental CHO cell-line. Table 9 shows the result of 7-day fed batch fermentations comparing the parental cell-line to the two 2,6-sialyltransferase clones. Sialic acid content was determined from the protein purified after the harvest. Four different IgMs were transfected into 2B4 and the parental cell-line and two of these IgMs were also transfected into 2C2.

TABLE 9
Harvest titer data and sialic acid levels on purified proteins from the harvested fermentation.

| | Clone 2B4 | | | | Clone 2C2 | | | |
|---|---|---|---|---|---|---|---|---|
| | | Fold | | Fold | | Fold | | Fold |
| | | Difference | | Difference in | | Difference | | Difference in |
| | | in Titer | Sialic | Sialic Acid | | in Titer | Sialic | Sialic Acid |
| | Titer | Relative to | Acid | Relative to | Titer | Relative to | Acid | Relative to |
| IgM | (mg/mL) | Parental | (mol/mol) | Parental | (mg/mL) | Parental | (mol/mol) | Parental |
| 1 | 2875 | 1.3 | 51.3 | 1.7 | | | | |
| 2 | 1078 | 1.6 | 46.0 | 1.5 | 379 | 0.6 | 37.2 | 1.2 |
| 3 | 752 | 1.6 | 41.4 | 1.6 | 727 | 1.5 | 40.0 | 1.5 |
| 4 | 1500 | 1.3 | 31.8 | 1.4 | | | | |

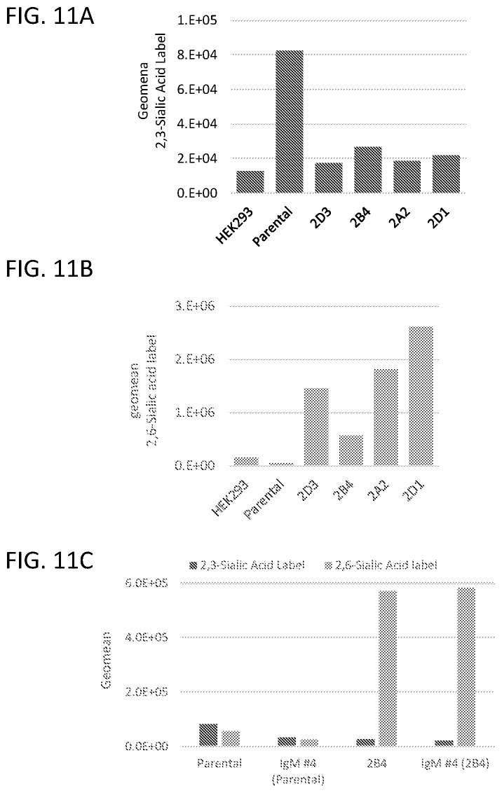

To further characterize the clones, 2,6-sialic acid and 2,3-sialic acid levels were measured on the cell surface by cytometry. Measurement was made using fluorescently conjugated lectins which are specific to either form of sialic acid. All viabilities were over 95% at the time the labeling was carried-out. HEK293 cells, the CHO parental cell-line, selected clones, and IgM transfected clones were all analyzed in an identical manner. FIGS. 11A and 11B show the 2,3-sialic acid and 2,6-sialic acid levels for untransfected cells, respectively. FIG. 11C compares the 2,3-sialic acid and 2,6-sialic acid levels in untransfected and IgM #4 transfected parental and 2B4 cells. The data shown in FIG. 11A indicates that the CHO parental cell had higher 2,3-sialic acid levels then HEK293 cells or the clones transfected with 2,6-sialyltransferase. The data shown in FIG. 11B indicates the 2,6-sialic acid levels on the clones was elevated well above the level of the CHO parental. FIG. 11C indicates that the IgM transfected cell-lines retained high levels of 2,6-sialic acid.

### Example 8

### In Vitro Sialylation Under Various Conditions

Varying amounts of truncated human ST6 were added to a reaction solution comprising an IgM antibody and cytidine-5/-monophospho-N-acetylneuraminic acid sodium salt (CMP-NANA, 1.5 mg/ml) in ratios as shown in Table 10. The durations and temperatures for each reaction, and the resulting sialylation (quantitated as described in Example 1) are also shown in Table 10. Room temperature (RT) is 15 to 25° C.

TABLE 10
in vitro sialylation conditions and results

| | | | | | Sialic |
|---|---|---|---|---|---|
| Condition | | Temp. | | Antibody:CMP- | Acid |
| # | Antibody in reaction | (° C.) | Time (h) | NANA:ST6 (mass) | (mol/mol) |
| 1 | anti-CD20 × CD3 IGM-A | 37 | 24 | — | 20 |
| 2 | anti-CD20 × CD3 IGM-A | 37 | 24 | 6:3:1 | 62 |
| 3 | Target 1 IGM-D | 37 | 6 | — | 27 |
| 4 | Target 1 IGM-D | 37 | 6 | 500:1000:1 | 47 |
| 5 | Target 2 IGM-E | 37 | 6 | — | 32 |
| 6 | Target 2 IGM-E | 37 | 6 | 100:50:1 | 62 |
| 7 | anti-CD20 × CD3 IGM-F | 37 | 7 | — | 20 |
| 8 | anti-CD20 × CD3 IGM-F | 37 | 7 | 100:50:1 | 54 |
| 9 | anti-CD20 × CD3 IGM-F | 37 | 7 | 100:50:1 | 64 |
| 10 | anti-CD20 × CD3 IGM-A | RT | 21 | 100:50:1 | 53 |
| 11 | anti-CD20 × CD3 IGM-A | RT | 21 | 100:25:1 | 51 |
| 12 | anti-CD20 × CD3 IGM-A | RT | 21 | 100:10:1 | 51 |
| 13 | anti-CD20 × CD3 IGM-A | RT | 21 | 1000:500:1 | 42 |
| 14 | anti-CD20 × CD3 IGM-A | RT | 21 | 1000:250:1 | 41 |
| 15 | anti-CD20 × CD3 IGM-A | RT | 21 | 1000:100:1 | 44 |
| 16 | anti-CD20 × CD3 IGM-A | RT | 20 | 100:50:1 | 48 |
| 17 | anti-CD20 × CD3 IGM-A | RT | 20 | 500:62.5:1 | 51 |
| 18 | anti-CD20 × CD3 IGM-A | RT | 20 | 1000:500:1 | 42 |
| 19 | anti-CD20 × CD3 IGM-A | RT | 20 | — | 16 |
| 20 | anti-CD20 × CD3 IGM-A | RT | 21 | 10000:5000:1 | 34 |
| 21 | anti-CD20 × CD3 IGM-A | RT | 21 | 3000:1500:1 | 39 |
| 22 | anti-CD20 × CD3 IGM-A | RT | 21 | 1000:500:1 | 42 |
| 23 | anti-CD20 × CD3 IGM-A | RT | 21 | 100:50:1 | 48 |
| 24 | anti-CD20 × CD3 IGM-A | 37 | 20 | — | 15 |
| 25 | anti-CD20 × CD3 IGM-A | 37 | 7 | 100:50:1 | 54 |
| 26 | anti-CD20 × CD3 IGM-A | RT | 20 | 100:50:1 | 59 |
| 27 | anti-CD20 × CD3 IGM-A | RT | 22 | 1000:50:1 | 52 |
| 28 | anti-CD20 × CD3 IGM-A | 4 | 8 | 100:50:1 | 53 |
| 29 | anti-CD20 × CD3 IGM-A | 15 | 48 | 100:50:1 | 54 |
| 30 | anti-CD20 × CD3 IGM-A | 37 | 24 | 100:50:1 | 50 |
| 31 | anti-CD20 × CD3 IGM-A | 37 | 24 | 5000:2500:1 | 45 |
| 32 | anti-CD20 × CD3 IGM-A | RT | 48 | 100:50:1 | 54 |
| 33 | anti-CD20 × CD3 IGM-A | RT | 48 | 250:125:1 | 50 |
| 34 | anti-CD20 × CD3 IGM-A | RT | 48 | 1000:500:1 | 45 |
| 35 | anti-CD20 × CD3 IGM-A | RT | 48 | 2500:1250:1 | 44 |
| 36 | anti-CD20 × CD3 IGM-A | RT | 48 | 5000:2500:1 | 40 |

The antibodies from Conditions 1 and 2 were compared by size exclusion chromatography (SEC), dynamic light scattering (DLS), hybrid gel, reducing gel and the CDC and TCA assays described in Example 3, and the in vitro sialyation did not change the SEC profile, the dynamic radius, or the mobility of the antibody, and the antibodies had similar activity TCA and CDC activity (data not shown).

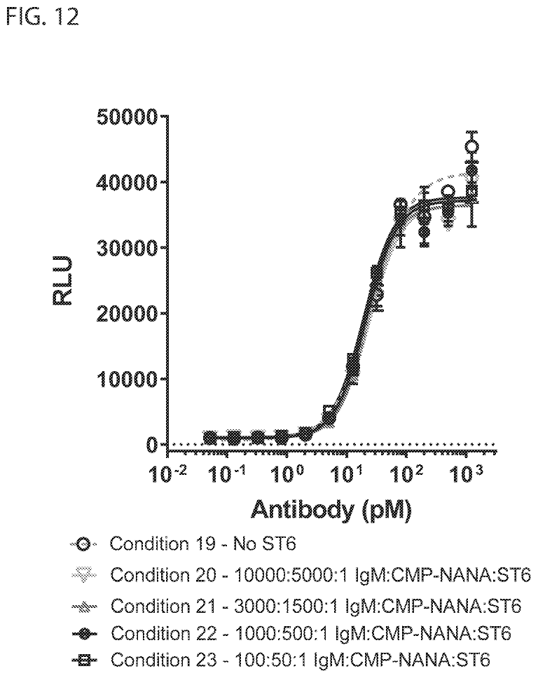

The antibodies from Conditions 19-23 were also compared by the TCA assay described in Example 3, and the data are shown in FIG. 12. All antibodies assayed had similar TCA activity.

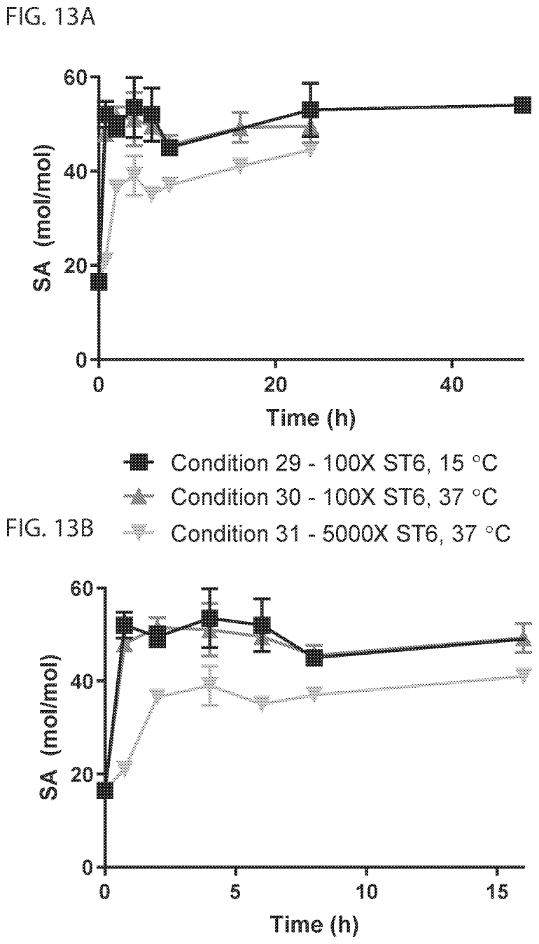

The SA levels of the antibodies from Conditions 29-31 were monitored at set timepoints throughout the reaction. The SA levels over 48 hours or over the first 15 hours are plotted in FIGS. 13A and 13B, respectively. For the antibody:CMP-NANA: ST-6 mass ratios of 100:50:1, maximum sialyation was reached with 1 hour and there was little difference observed between 37° C. and 15° C.

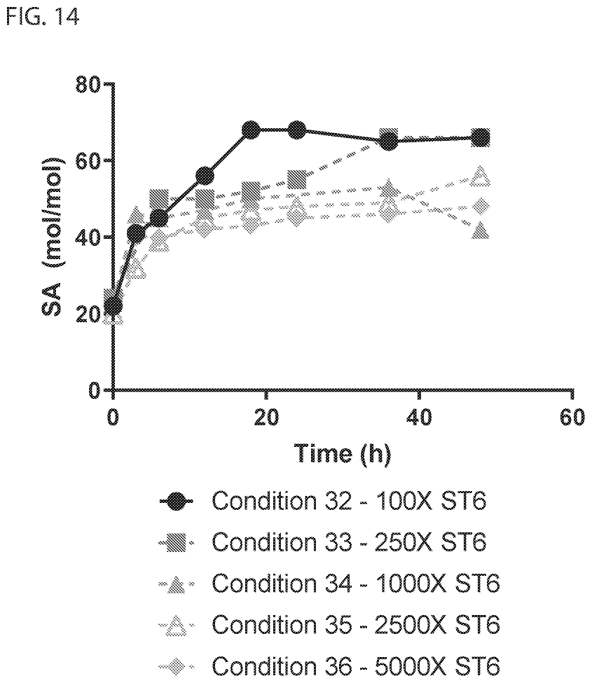

The SA levels of the antibodies from Conditions 32-36 were monitored at set timepoints throughout the reaction. The SA levels over 48 hours are plotted in FIG. 14. Antibody:CMP-NANA:ST-6 mass ratios of 100:50:1 achieved sialyation of >60 mol/mol SA by 18 h at RT and did not drop significantly for up to 48 hours at RT. Antibody:CMP-NANA:ST-6 mass ratios of 250:125:1 resulted in a slower rise in SA levels and reached >60 mol/mol SA after 36 h. For all ratios, no significant desialylation was observed at RT up to 36 hrs. At antibody:CMP-NANA:ST-6 mass ratios of 5000:2500:1, 40 mol/mol SA was achieved, and the antibody population was not sialylated to the maximum extent possible.

### Example 9

### Pharmacokinetics of IgM Antibodies With High Sialic Acid Levels

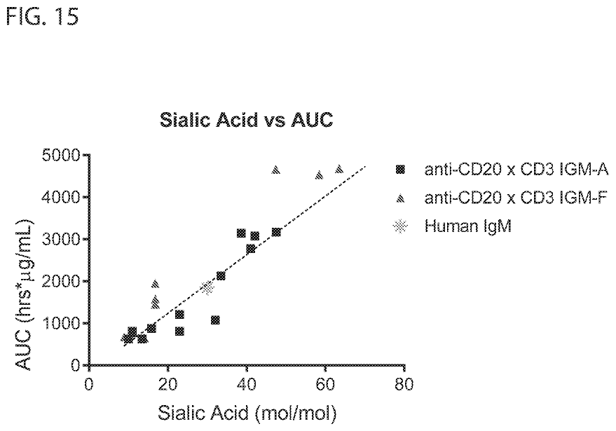

Pharmacokinetic parameters were measured for various IgM antibodies in an in vivo mouse model as follows. Balb/c mice were injected with 5 mg/kg of anti-CD20×CD3 IGM-A, anti-CD20×CD3 IGM-A-GEM antibodies of various sialic acid levels, anti-CD20×CD3 IGM-F, anti-CD20×CD3 IGM-F-GEM antibodies of various sialic acid levels, or human serum IgM via intravenous infusion. Blood samples were collected at 10 or 12 time points total for each antibody, with at least 2 mice per time point. Each mouse was bled once through the facial vein (100 μL) and then another time by terminal cardiac puncture (max obtainable, ˜500 μL). A standard ELISA assay was used to measure the serum concentration of each antibody in the blood at each time point. Quality metrics were verified on all ELISAs, and PK parameters, including T_{1/2-alpha}, T_{1/2-beta}, and the area under the concentration curve from time zero to infinity (AUC_{0-∞}, measured in μg/ml*hr) were derived using standard curve fitting techniques (Win Non Lin, Phoenix Software). The sialic acid levels and the resulting AUG-_{0-∞} for each antibody are shown in FIG. 15.

### Example 10

### IgM Antibodies with High Sialic Acid Levels Activity in Cynomolgus Monkeys

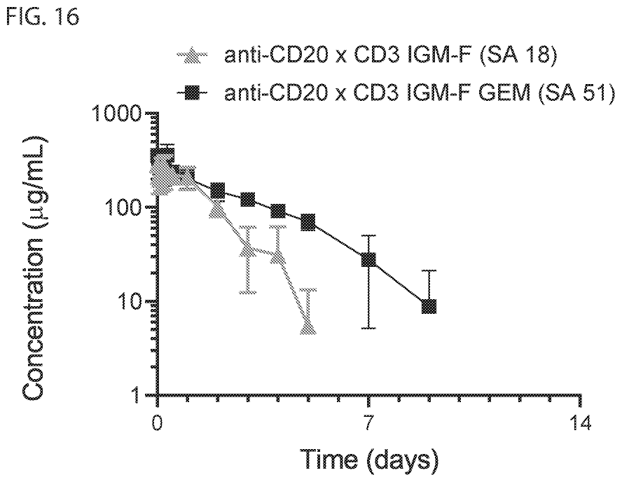

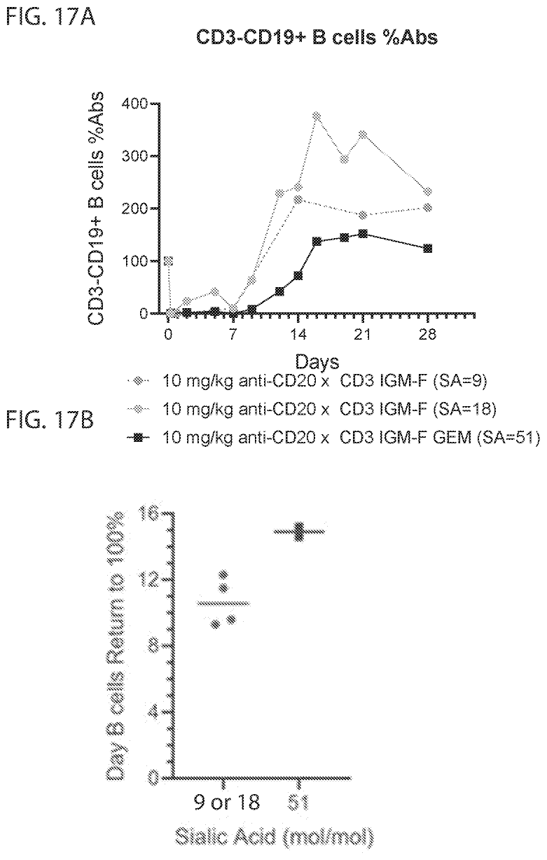

Pharmacokinetic parameters and cellular markers were measured for IgM antibodies in an in vivo cynomolgus monkey model as follows. Cynomolgus primates were injected with 10 mg/kg of anti-CD20×CD3 IGM-F (SA 18 mol/mol) (2 animals), anti-CD20×CD3 IGM-F (SA 9 mol/mol) (2 animals), or anti-CD20×CD3 IGM-F-GEM (SA 51 mol/mol) (4 animals) antibodies. Blood samples were collected at 12 time points total for each antibody. A standard ELISA assay was used to measure the serum concentration of each antibody in the blood at each time point. Quality metrics were verified on all ELISAs, and PK parameters, including T_{1/2-alpha}, T_{1/2-beta}, and the area under the concentration curve from time zero to infinity (AUC_{0-∞}, measured in μg/ml*hr) were derived using standard curve fitting techniques (Win Non Lin, Phoenix Software). Flow cytometry was used to measure cellular markers. The PK results for anti-CD20×CD3 IGM-F (SA 18 mol/mol) and 2 of the 4 anti-CD20×CD3 IGM-F-GEM (SA 51 mol/mol) treated animals are presented in FIG. 16. The AUG_{0-∞} for the high sialic acid antibody was twofold higher than for the low sialic acid antibody. The relative numbers of B cells at each time point are shown in FIG. 17A, and the day that B cells began to recover is shown in FIG. 17B.

### Example 11

### In Vitro Sialylation with Multiple Enzymes

The combination of galactosylation and sialylation was compared to sialylation only.

Sialylation only was completed by mixing 120 μg anti-CD20×CD3 IGM-A antibody with 1 mol/mol SA or 21 mol/mol SA, 60 μg of CMP NANA, and 20 μg ST6 (a mass ratio of IgM:CMP NANA: ST6 of 6:3:1). The sample was then incubated for 24 hours at 37° C.

The combination of galactosylation and sialylation was completed by mixing 1 μg β-1,4-Galactosyltransferase, 60 μg UDP-Galactose, 60 μg anti-CD20×CD3 IGM-A antibody with 1 mol/mol SA or 21 mol/mol SA (a mass ratio of IgM: UDP-Galactose: β-1,4-Galactosyltransferase of 60:60:1). The mixture was incubated for 7 hours at 37° C. 30 μg CMP NANA and 1 μg of ST6 were then added to the mixture and was incubated for 20 hours at 37° C.

The resulting sialic acid levels are shown in Table 11

TABLE 11
Sialic levels after sialylation with or without galactosylation

| Starting | | Sialic Acid |
|---|---|---|
| Material | Treatment | (mol/mol) |
| anti-CD20 × CD3 IGM-A | Sialylation | 44 |
| 1 mol/mol SA | only | |
| anti-CD20 × CD3 IGM-A | Galactosylation | 65 |
| 1 mol/mol SA | and Sialylation | |
| anti-CD20 × CD3 IGM-A | Sialylation | 60 |
| 21 mol/mol SA | only | |
| anti-CD20 × CD3 IGM-A | Galactosylation | 65 |
| 21 mol/mol SA | and Sialylation | |

The breadth and scope of the present disclosure should not be limited by any of the above-described exemplary embodiments or examples but should be defined only in accordance with the following claims and their equivalents.

TABLE 12
Sequences in the Disclosure

| SEQ | Nickname | |
|---|---|---|
| ID | (source) | Sequence |
| 1 | Human IgM | GSASAPTLFPLVSCENSPSDTSSVAVGCLAQDFLPDSITFSWKYKNNSDISSTRGFPS |
| | Constant | VLRGGKYAATSQVLLPSKDVMQGTDEHVVCKVQHPNGNKEKNVPLPVIAELPPKVSVF |
| | region IMGT | VPPRDGFFGNPRKSKLICQATGFSPRQIQVSWLREGKQVGSGVTTDQVQAEAKESGPT |
| | allele | TYKVTSTLTIKESDWLSQSMFTCRVDHRGLTFQQNASSMCVPDQDTAIRVFAIPPSFA |
| | IGHM+03 | SIFLTKSTKLTCLVTDLTTYDSVTISWTRQNGEAVKTHTNISESHPNATFSAVGEASI |
| | (GenBank: | CEDDWNSGERFTCTVTHTDLPSPLKQTISRPKGVALHRPDVYLLPPAREQLNLRESAT |
| | pir\|S37768\|) | ITCLVTGFSPADVFVQWMQRGQPLSPEKYVTSAPMPEPQAPGRYFAHSILTVSEEEWN |
| | | TGETYTCVVAHEALPNRVTERTVDKSTGKPTLYNVSLVMSDTAGTCY |
| 2 | Human IgM | GSASAPTLFPLVSCENSPSDTSSVAVGCLAQDFLPDSITFSWKYKNNSDISSTRGFPS |
| | Constant | VLRGGKYAATSQVLLPSKDVMQGTDEHVVCKVQHPNGNKEKNVPLPVIAELPPKVSVF |
| | region IMGT | VPPRDGFFGNPRKSKLICQATGFSPRQIQVSWLREGKQVGSGVTTDQVQAEAKESGPT |
| | allele | TYKVTSTLTIKESDWLGQSMFTCRVDHRGLTFQQNASSMCVPDQDTAIRVFAIPPSFA |
| | IGHM*04 | SIFLTKSTKLTCLVTDLTTYDSVTISWTRQNGEAVKTHTNISESHPNATFSAVGEASI |
| | (GenBank: | CEDDWNSGERFTCTVTHTDLPSPLKQTISRPKGVALHRPDVYLLPPAREQLNLRESAT |
| | sp\|P01871.41) | ITCLVTGFSPADVFVQWMQRGQPLSPEKYVTSAPMPEPQAPGRYFAHSILTVSEEEWN |
| | | TGETYTCWAHEALPNRVTERTVDKSTGKPTLYNVSLVMSDTAGTCY |
| 3 | Human ST6Gal | MIHTNLKKKFSCCVLVFLLFAVICVWKEKKKGSYYDSFKLQTKEFQVLKSLGKLAMGS |
| | I, | DSQSVSSSSTQDPHRGRQTLGSLRGLAKAKPEASFQVWNKDSSSKNLIPRLQKIWKNY |
| | Sialyl- | LSMNKYKVSYKGPGPGIKFSAEALRCHLRDHVNVSMVEVTDFPFNTSEWEGYLPKESI |
| | transferase 1 | RTKAGPWGRCAVVSSAGSLKSSQLGREIDDHDAVLRFNGAPTANFQQDVGTKTTIRLM |
| | (GenBank: | NSQLVTTEKRFLKDSLYNEGILIVWDPSVYHSDIPKWYQNPDYNFFNNYKTYRKLHPN |
| | sp\|P15907.11) | QPFYILKPQMPWELWDILQEISPEEIQPNPPSSGMLGIIIMMTLCDQVDIYEFLPSKR |
| | | KTDVCYYYQKFFDSACTMGAYHPLLYEKNLVKHLNQGTDEDIYLLGKATLPGFRTIHC |
| 4 | Human | MGFNLTFHLSYKFRLLLLLTLCLTVVGWATSNYFVGAIQEIPKAKEFMANFHKTLILG |
| | B4GALT4, | KGKTLTNEASTKKVELDNCPSVSPYLRGQSKLIFKPDLTLEEVQAENPKVSRGRYRPQ |
| | HUMAN Beta- | ECKALQRVAILVPHRNREKHLMYLLEHLHPFLQRQQLDYGIYVIHQAEGKKFNRAKLL |
| | 1, 4- | NVGYLEALKEENWDCFIFHDVDLVPENDFNLYKCEEHPKHLVVGRNSTGYRLRYSGYF |
| | galactosyl- | GGVTALSREQFFKVNGFSNNYWGWGGEDDDLRLRVELQRMKISRPLPEVGKYTMVFHT |
| | transferase | RDKGNEVNAERMKLLHQVSRVWRTDGLSSCSYKLVSVEHNPLYINITVDFWFGA |
| | (GenBank: | |
| | sp\|O60513\|) | |
| 5 | Precursor | MKNHLLFWGVLAVFIKAVHVKAQEDERIVLVDNKCKCARITSRIIRSSEDPNEDIVER |
| | Human J Chain | NIRIIVPLNNRENISDPTSPLRTRFVYHLSDLCKKCDPTEVELDNQIVTATQSNICDE |
| | | DSATETCYTYDRNKCYTAVVPLVYGGETKMVETALTPDACYPD |
| 6 | Mature Human | QEDERIVLVDNKCKCARITSRIIRSSEDPNEDIVERNIRIIVPLNNRENISDPTSPLR |
| | J Chain | TRFVYHLSDLCKKCDPTEVELDNQIVTATQSNICDEDSATETCYTYDRNKCYTAVVPL |
| | | VYGGETKMVETALTPDACYPD |
| 7 | J Chain Y102A | QEDERIVLVDNKCKCARITSRIIRSSEDPNEDIVERNIRIIVPLNNRENISDPTSPLR |
| | mutation | TRFVYHLSDLCKKCDPTEVELDNQIVTATQSNICDEDSATETCATYDRNKCYTAVVPL |
| | | VYGGETKMVETALTPDACYPD |
| 8 | anti-CD20 x | EVQLVQSGAEVKKPGESLKISCKGSGYSFTSYWIGWVRQMPGKGLEWMGIIYPGDSDT |
| | CD3 IGM-A VH | RYSPSFQGQVTISADKSITTAYLQWSSLKASDTAMYYCARHPSYGSGSPNFDYWGQGT |
| | | LVTVSS |
| 9 | anti-CD20 x | DIVMTQTPLSSPVTLGQPASISCRSSQSLVYSDGNTYLSWLQQRPGQPPRLLIYKISN |
| | CD3 IGM-A VL | RFSGVPDRFSGSGAGTDFTLKISRVEAEDVGVYYCVQATQFPLTFGGGTKVEIK |
| 10 | anti-DR5 IgM- | EVQLVESGGGLVQPGGSLRLSCAASGFTFSSYVMSWVRQAPGKGLEWVATISSGGSYT |
| | B VH | YYPDSVKGRFTISRDNAKNTLYLQMNSLRAEDTAVYYCARRGDSMITTDYWGQGTLVT |
| | | VSSA |
| 11 | anti-DR5 IgM- | DIQMTQSPSSLSASVGDRVTITCKASQDVGTAVAWYQQKPGKAPKLLIYWASTRHTGV |
| | B VL | PSRFSGSGSGTDFTLTISSLQPEDFATYYCQQYSSYRTFGQGTKVEIK |
| 12 | anti-DR5 IgM- | QVQLQESGPGLVKPSQTLSLTCTVSGGSISSGDYFWSWIRQLPGKGLECIGHIHNSGT |
| | C VH | TYYNPSLKSRVTISVDTSKKQFSLRLSSVTAADTAVYYCARDRGGDYYYGMDVWGQGT |
| | | TVTVSS |
| 13 | anti-DR5 IgM- | EIVLTQSPGTLSLSPGERATLSCRASQGISRSYLAWYQQKPGQAPSLLIYGASSRATG |
| | C VL | IPDRFSGSGSGTDFTLTISRLEPEDFAVYYCQQFGSSPWTFGQGTKVEIK |
| 14 | SP34 VH | EVQLVESGGGLVQPKGSLKLSCAASGFTFNTYAMNWVRQAPGKGLEWVARIRSKYNNY |
| | | ATYYADSVKDRFTISRDDSQSILYLQMNNLKTEDTAMYYCVRHGNFGNSYVSWFAYWG |
| | | QGTLVTVSS |
| 15 | SP34 VH CDR1 | GFTFNTYAMN |
| 16 | SP34 VH CDR2 | ARIRSKYNNYATYYADSVKD |
| 17 | SP34 VH CDR3 | VRHGNFGNSYVSWFAY |
| 18 | SP34 VL | QAVVTQESALTTSPGETVTLTCRSSTGAVTTSNYANWVQEKPDHLFTGLIGGTNKRAP |
| | | GVPARFSGSLIGDKAALTITGAQTEDEAIYFCALWYSNLWVFGGGTKLTVL |
| 19 | SP34 VL CDR1 | RSSTGAVTTSNYAN |
| 20 | SP34 VL CDR2 | GTNKRAP |
| 21 | SP34 VL CDR3 | ALWYSNL |
| 22 | A-55-VH | EVQLLESGGGLVQPGGSLRLSCAASGFTFDTYAMNWVRQAPGKGLEWVARIRSKYNNY |
| | (WO2018208864) | ATYYADSVKDRFTISRDDSKSTLYLQMESLRAEDTAVYYCVRHGNFQGGYVSWFAHWG |
| | | QGTLVTVSS |
| 23 | A-55-VL | QTVVTQEPSLSVSPGGTVTLTCGSSTGAVTTSNYANWVQQTPGQAPRGLIGGTDKRAP |
| | (WO2018208864) | GVPDRFSGSLLGDKAALTITGAQAEDEADYYCALWYSNHWVFGGGTKLTVL |
| 24 | A-55-scFv | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHGNFQGGYVSWFAHWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVL |
| 25 | A-56-VH | EVQLLESGGGLVQPGGSLRLSCAASGFTFDTYAMNWVRQAPGKGLEWVARIRSKYNNY |
| | (WO2018208864) | ATYYADSVKDRFTISRDDSKSTLYLQMESLRAEDTAVYYCVRHGNFGGGYVSWFAWWG |
| | | QGTLVTVSS |
| 26 | A-56-V | QTVVTQEPSLSVSPGGTVTLTCGSSTGAVTTSNYANWVQQTPGQAPRGLIGGTDKRAP |
| | (WO2018208864) | GVPDRFSGSLLGDKAALTITGAQAEDEADYYCALWYSNHWVFGGGTKLTVL |
| 27 | A-56-scFv | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHGNFGGGYVSWFAWWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVL |
| 28 | A-57-VH | EVQLLESGGGLVQPGGSLRLSCAASGFTFDTYAMNWVRQAPGKGLEWVARIRSKYNNY |
| | (WO2018208864) | ATYYADSVKDRFTISRDDSKSTLYLQMESLRAEDTAVYYCVRHANFGAGYVSWFAHWG |
| | | QGTLVTVSS |
| 29 | A-57-VL | QTVVTQEPSLSVSPGGTVTLTCGSSTGAVTTSNYANWVQQTPGQAPRGLIGGTDKRAP |
| | (WO2018208864) | GVPDRFSGSLLGDKAALTITGAQAEDEADYYCALWYSNHWVFGGGTKLTVL |
| 30 | A-57-scFv | >Adi26957_scFv |
| | | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHANFGAGYVSWFAHWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVL |
| 31 | A-55-J* | >X26955J(Y102A) |
| | | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHGNFQGGYVSWFAHWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVLGGGGSGGGGSGGGGSQEDERIVLVDNKCKCARITSRIIRSS |
| | | EDPNEDIVERNIRIIVPLNNRENISDPTSPLRTRFVYHLSDLCKKCDPTEVELDNQIV |
| | | TATQSNICDEDSATETCATYDRNKCYTAVVPLVYGGETKMVETALTPDACYPD |
| 32 | A-56-J* | >X26956J(Y102A) |
| | | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHGNFGGGYVSWFAWWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVLGGGGSGGGGSGGGGSQEDERIVLVDNKCKCARITSRIIRSS |
| | | EDPNEDIVERNIRIIVPLNNRENISDPTSPLRTRFVYHLSDLCKKCDPTEVELDNQIV |
| | | TATQSNICDEDSATETCATYDRNKCYTAVVPLVYGGETKMVETALTPDACYPD* |
| 33 | A-57-J* | >X26957J(Y102A) |
| | | EVQLLESGGGLVQPGGSLRLSCAASGFTFDTYAMNWVRQAPGKGLEWVARIRSKYNNY |
| | | ATYYADSVKDRFTISRDDSKSTLYLQMESLRAEDTAVYYCVRHANFGAGYVSWFAHWG |
| | | QGTLVTVSSGGGGSGGGGSGGGGSQTVVTQEPSLSVSPGGTVTLTCGSSTGAVTTSNY |
| | | ANWVQQTPGQAPRGLIGGTDKRAPGVPDRFSGSLLGDKAALTITGAQAEDEADYYCAL |
| | | WYSNHWVFGGGTKLTVLGGGGSGGGGSGGGGSQEDERIVLVDNKCKCARITSRIIRSS |
| | | EDPNEDIVERNIRIIVPLNNRENISDPTSPLRTRFVYHLSDLCKKCDPTEVELDNQIV |
| | | TATQSNICDEDSATETCATYDRNKCYTAWPLVYGGETKMVETALTPDACYPD* |
| 34 | VJH (WT J) | MGWSYIILFLVATATGVHSQVQLVQSGAEVKKPGASVKVSCKASGYTFISYTMHWVRQ |
| | | APGQGLEWMGYINPRSGYTHYNQKLKDKATLTADKSASTAYMELSSLRSEDTAVYYCA |
| | | RSAYYDYDGFAYWGQGTLVTVSSGGGGSGGGGSGGGGSDIQMTQSPSSLSASVGDRVT |
| | | ITCSASSSVSYMNWYQQKPGKAPKRLIYDTSKLASGVPSRFSGSGSGTDFTLTISSLQ |
| | | PEDFATYYCQQWSSNPPTFGGGTKVEIKGGGGSGGGGSGGGGSQEDERIVLVDNKCKC |
| | | ARITSRIIRSSEDPNEDIVERNIRIIVPLNNRENISDPTSPLRTRFVYHLSDLCKKCD |
| | | PTEVELDNQIVTATQSNICDEDSATETCYTYDRNKCYTAVVPLVYGGETKMVETALTP |
| | | DACYPDGGGGSGGGGSGGGGSDAHKSEVAHRFKDLGEENFKALVLIAFAQYLQQCPFE |
| | | DHVKLVNEVTEFAKTCVADESAENCDKSLHTLFGDKLCTVATLRETYGEMADCCAKQE |
| | | PERNECFLQHKDDNPNLPRLVRPEVDVMCTAFHDNEETFLKKYLYEIARRHPYFYAPE |
| | | LLFFAKRYKAAFTECCQAADKAACLLPKLDELRDEGKASSAKQRLKCASLQKFGERAF |
| | | KAWAVARLSQRFPKAEFAEVSKLVTDLTKVHTECCHGDLLECADDRADLAKYICENQD |
| | | SISSKLKECCEKPLLEKSHCIAEVENDEMPADLPSLAADFVESKDVCKNYAEAKDVFL |
| | | GMFLYEYARRHPDYSVVLLLRLAKTYETTLEKCCAAADPHECYAKVFDEFKPLVEEPQ |
| | | NLIKQNCELFKQLGEYKFQNALLVRYTKKVPQVSTPTLVEVSRNLGKVGSKCCKHPEA |
| | | KRMPCAEDYLSVVLNQLCVLHEKTPVSDRVTKCCTESLVNRRPCFSALEVDETYVPKE |
| | | FNAETFTFHADICTLSEKERQIKKQTALVELVKHKPKATKEQLKAVMDDFAAFVEKCC |
| | | KADDKETCFAEEGKKLVAASQAALGL |
| 35 | VJ*H (WT J) | MGWSYIILFLVATATGVHSQVQLVQSGAEVKKPGASVKVSCKASGYTFISYTMHWVRQ |
| | | APGQGLEWMGYINPRSGYTHYNQKLKDKATLTADKSASTAYMELSSLRSEDTAVYYCA |
| | | RSAYYDYDGFAYWGQGTLVTVSSGGGGSGGGGSGGGGSDIQMTQSPSSLSASVGDRVT |
| | | ITCSASSSVSYMNWYQQKPGKAPKRLIYDTSKLASGVPSRFSGSGSGTDFTLTISSLQ |
| | | PEDFATYYCQQWSSNPPTFGGGTKVEIKGGGGSGGGGSGGGGSQEDERIVLVDNKCKC |
| | | ARITSRIIRSSEDPNEDIVERNIRIIVPLNNRENISDPTSPLRTRFVYHLSDLCKKCD |
| | | PTEVELDNQIVTATQSNICDEDSATETCATYDRNKCYTAVVPLVYGGETKMVETALTP |
| | | DACYPDGGGGSGGGGSGGGGSDAHKSEVAHRFKDLGEENFKALVLIAFAQYLQQCPFE |
| | | DHVKLVNEVTEFAKTCVADESAENCDKSLHTLFGDKLCTVATLRETYGEMADCCAKQE |
| | | PERNECFLQHKDDNPNLPRLVRPEVDVMCTAFHDNEETFLKKYLYEIARRHPYFYAPE |
| | | LLFFAKRYKAAFTECCQAADKAACLLPKLDELRDEGKASSAKQRLKCASLQKFGERAF |
| | | KAWAVARLSQRFPKAEFAEVSKLVTDLTKVHTECCHGDLLECADDRADLAKYICENQD |
| | | SISSKLKECCEKPLLEKSHCIAEVENDEMPADLPSLAADFVESKDVCKNYAEAKDVFL |
| | | GMFLYEYARRHPDYSVVLLLRLAKTYETTLEKCCAAADPHECYAKVFDEFKPLVEEPQ |
| | | NLIKQNCELFKQLGEYKFQNALLVRYTKKVPQVSTPTLVEVSRNLGKVGSKCCKHPEA |
| | | KRMPCAEDYLSVVLNQLCVLHEKTPVSDRVTKCCTESLVNRRPCFSALEVDETYVPKE |
| | | FNAETFTFHADICTLSEKERQIKKQTALVELVKHKPKATKEQLKAVMDDFAAFVEKCC |
| | | KADDKETCFAEEGKKLVAASQAALGL |
| 36 | mature V15J | QVQLVQSGAEVKKPGASVKVSCKASGYTFISYTMHWVRQAPGQGLEWMGYINPRSGYT |
| | (″WT″, or | HYNQKLKDKATLTADKSASTAYMELSSLRSEDTAVYYCARSAYYDYDGFAYWGQGTLV |
| | wild-type) | TVSSGGGGSGGGGSGGGGSDIQMTQSPSSLSASVGDRVTITCSASSSVSYMNWYQQKP |
| | | GKAPKRLIYDTSKLASGVPSRFSGSGSGTDFTLTISSLQPEDFATYYCQQWSSNPPTF |
| | | GGGTKLEIKGGGGSGGGGSGGGGSQEDERIVLVDNKCKCARITSRIIRSSEDPNEDIV |
| | | ERNIRIIVPLNNRENISDPTSPLRTRFVYHLSDLCKKCDPTEVELDNQIVTATQSNIC |
| | | DEDSATETCYTYDRNKCYTAVVPLVYGGETKMVETALTPDACYPD |
| 37 | mature V15J* | QVQLVQSGAEVKKPGASVKVSCKASGYTFISYTMHWVRQAPGQGLEWMGYINPRSGYT |
| | | HYNQKLKDKATLTADKSASTAYMELSSLRSEDTAVYYCARSAYYDYDGFAYWGQGTLV |
| | | TVSSGGGGSGGGGSGGGGSDIQMTQSPSSLSASVGDRVTITCSASSSVSYMNWYQQKP |
| | | GKAPKRLIYDTSKLASGVPSRFSGSGSGTDFTLTISSLQPEDFATYYCQQWSSNPPTF |
| | | GGGTKLEIKGGGGSGGGGSGGGGSQEDERIVLVDNKCKCARITSRIIRSSEDPNEDIV |
| | | ERNIRIIVPLNNRENISDPTSPLRTRFVYHLSDLCKKCDPTEVELDNQIVTATQSNIC |
| | | DEDSATETCATYDRNKCYTAVVPLVYGGETKMVETALTPDACYPD |
| 38 | SJ* | >SJ* (S = anti CD3 scfv SP34) |
| | | MGWSYIILFLVATATGVHSEVQLVESGGGLVQPKGSLKLSCAASGFTFNTYAMNWVRQ |
| | | APGKGLEWVARIRSKYNNYATYYADSVKDRFTISRDDSQSILYLQMNNLKTEDTAMYY |
| | | CVRHGNFGNSYVSWFAYWGQGTLVTVSSGGGGSGGGGSGGGGSQAVVTQESALTTSPG |
| | | ETVTLTCRSSTGAVTTSNYANWVQEKPDHLFTGLIGGTNKRAPGVPARFSGSLIGDKA |
| | | ALTITGAQTEDEAIYFCALWYSNLWVFGGGTKLTVLGGGGSGGGGSGGGGSQEDERIV |
| | | LVDNKCKCARITSRIIRSSEDPNEDIVERNIRIIVPLNNRENISDPTSPLRTRFVYHL |
| | | SDLCKKCDPTEVELDNQIVTATQSNICDEDSATETCATYDRNKCYTAVVPLVYGGETK |
| | | MVETALTPDACYPD |
| 39 | ST6 QPCR | GACCGACGTGTGCTACTATTAC |
| | primer 1 | |
| 40 | ST6 QPCR | GAGGTGCTTCACGAGATTCTT |
| | Primer 2 | |
| 41 | ″5″ Peptide | GGGGS |
| | linker | |
| 42 | ″10″ Peptide | GGGGSGGGGS |
| | linker | |
| 43 | ″15″ Peptide | GGGGSGGGGSGGGGS |
| | linker | |
| 44 | ″20″ Peptide | GGGGSGGGGSGGGGSGGGGS |
| | linker | |
| 45 | ″25″ Peptide | GGGGSGGGGSGGGGSGGGGSGGGGS |
| | Linker | |
| 46 | mouse IgM | ASQSFPNVFPLVSCESPLSDKNLVAMGCLARDFLPSTISFTWNYQNNTEVIQGIRTFP |
| | heavy chain | TLRTGGKYLATSQVLLSPKSILEGSDEYLVCKIHYGGKNRDLHVPIPAVAEMNPNVNV |
| | constant | FVPPRDGFSGPAPRKSKLICEATNFTPKPITVSWLKDGKLVESGFTTDPVTIENKGST |
| | region | PQTYKVISTLTISEIDWLNLNVYTCRVDHRGLTFLKNVSSTCAASPSTDILNFTIPPS |
| | (CAC20701.1) | FADIFLSKSANLTCLVSNLATYETLSISWASQSGEPLETKIKIMESHPNGTFSAKGVA |
| | | SVCVEDWNNRKEFVCTVTHRDLPSPQKKFISKPNEVHKHPPAVYLLPPAREQLNLRES |
| | | ATVTCLVKGFSPADISVQWKQRGQLLPQEKYVTSAPMPEPGAPGFYFTHSILTVTEEE |
| | | WNSGETYTCVVGHEALPHLVTERTVDKSTGKPTLYNVSLIMSDTGGTCY |
| 47 | Cynomolgus | ESAGPFKWEPSVSSPNAPLDTNEVAVGCLAQDFLPDSITFSWKFKNNSDISKGVWGFP |
| | Monkey | SVLRGGKYAATSQVLLASKDVMQGTDEHVVCKVQHPNGNKEQNVPLPVVAERPPNVSV |
| | presumed IgM | FVPPRDGFVGNPRESKLICQATGFSPRQIEVSWLRDGKQVGSGITTDRVEAEAKESGP |
| | constant | TTFKVTSTLTVSERDWLSQSVFTCRVDHRGLTFQKNVSSVCGPNPDTAIRVFAIPPSF |
| | region | ASIFLTKSTKLTCLVTDLATYDSVTITWTRQNGEALKTHTNISESHPNGTFSAVGEAS |
| | sequence | ICEDDWNSGERFRCTVTHTDLPSPLKQTISRPKGVAMHRPDVYLLPPAREQLNLRESA |
| | (EHH62210.1) | TITCLVTGFSPADIFVQWMQRGQPLSPEKYVTSAPMPEPQAPGRYFAHSILTVSEEDW |
| | | NTGETYTCVVAHEALPNRVTERTVDKSTGKPTLYNVSLVILWTTLSTFVALFVLTLLY |
| | | SGIVTFIKVR |

1. A monoclonal population of multimeric binding molecules, each binding molecule comprising ten or twelve IgM-derived heavy chains, wherein the IgM-derived heavy chains comprise glycosylated IgM heavy chain constant regions each associated with a binding domain that specifically binds to a target, wherein each IgM heavy chain constant region comprises at least one asparagine (N)-linked glycosylation motifs, wherein an N-linked glycosylation motif comprises the amino acid sequence N-X1-S/T, wherein N is asparagine, X1 is any amino acid except proline, and S/T is serine or threonine, wherein at least one of the N-linked glycosylation motifs on each IgM heavy chain constant region are occupied by a complex glycan, and wherein the monoclonal population of binding molecules comprises at least thirty-five (35) moles sialic acid per mole of the binding molecules.

2. The monoclonal population of binding molecules of claim 1, comprising about 40 to about 70 moles sialic acid per mole of binding molecule.

3. The monoclonal population of binding molecules of claim 1, wherein the IgM heavy chain constant regions are human IgM heavy chain constant regions or variants thereof comprising five N-linked glycosylation motifs N-X1-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04).

4-9. (canceled)

10. The monoclonal population of binding molecules of claim 1, wherein each binding molecule is a pentameric or a hexameric IgM antibody comprising five or six bivalent IgM binding units, respectively, wherein each binding unit comprises two IgM heavy chains each comprising a VH situated amino terminal to the variant IgM constant region, and two immunoglobulin light chains each comprising a light chain variable domain (VL) situated amino terminal to an immunoglobulin light chain constant region, and wherein the VH and VL combine to form an antigen-binding domain that specifically binds to the target.

11. The monoclonal population of binding molecules of claim 10, wherein each binding molecule is pentameric and further comprises a J-chain, or functional fragment thereof, or a functional variant thereof.

12. The monoclonal population of binding molecules of claim 11, wherein the J-chain is a mature human J-chain comprising the amino acid sequence SEQ ID NO: 6 or a functional fragment thereof, or a functional variant thereof.

13. The monoclonal population of binding molecules of claim 11, wherein the variant J-chain or functional fragment thereof comprises an amino acid substitution at the amino acid position corresponding to amino acid Y102 of the wild-type mature human J-chain of SEQ ID NO: 6.

14. The monoclonal population of binding molecules of claim 13, wherein the amino acid corresponding to Y102 of SEQ ID NO: 6 is substituted with alanine (A).

15. The monoclonal population of binding molecules of claim 14, wherein the J-chain comprises the amino acid sequence SEQ ID NO: 7.

16. The monoclonal population of binding molecules of claim 11, wherein the J-chain or fragment or variant thereof is a modified J-chain further comprising a heterologous moiety, wherein the heterologous moiety is fused or conjugated to the J-chain or fragment or variant thereof.

17. The monoclonal population of binding molecules of claim 16, wherein the heterologous moiety is a polypeptide fused to the J-chain or fragment or variant thereof.

18. The monoclonal population of binding molecules of claim 17, wherein the heterologous polypeptide is fused to the J-chain or fragment or variant thereof via a peptide linker comprising at least 5 amino acids, but no more than 25 amino acids.

19. (canceled)

20. The monoclonal population of binding molecules of claim 17, wherein the heterologous polypeptide comprises a scFv fragment.

21. The monoclonal population of binding molecules of claim 20, wherein the heterologous scFv fragment binds to CD3ε.

22. A pharmaceutical composition comprising the monoclonal population of binding molecules of claim 1 and a pharmaceutically acceptable excipient.

23. A recombinant host cell that produces the monoclonal population of binding molecules of claim 1.

24. (canceled)

25. A method for producing a monoclonal population of highly sialylated multimeric binding molecules, comprising providing a cell line that expresses the monoclonal population of binding molecules, culturing the cell line, and recovering the monoclonal population of binding molecules, wherein each binding molecule comprises ten or twelve IgM-derived heavy chains, wherein the IgM-derived heavy chains comprise glycosylated IgM heavy chain constant regions each associated with a binding domain that specifically binds to a target, wherein each IgM heavy chain constant region comprises at least three asparagine(N)-linked glycosylation motifs, wherein an N-linked glycosylation motif comprises the amino acid sequence N-Xi-S/T, wherein N is asparagine, X₁ is any amino acid except proline, and S/T is serine or threonine, wherein on average at least one of the N-linked glycosylation motifs on each IgM heavy chain constant region in the population are occupied by complex glycans, and wherein the cell line, recovery process, or a combination thereof is optimized to enrich for complex glycans comprising at least one sialic acid terminal monosaccharides per glycan.

26. The method of claim 25, wherein the cell line, recovery process, or a combination thereof is optimized to result in a monoclonal population of binding molecules comprising at least 30 moles sialic acid per mole of binding molecule; or comprising about 40 to about 70 moles sialic acid per mole of binding molecule.

27. The method of claim 25, wherein the IgM heavy chain constant regions are derived from human IgM heavy chain constant regions comprising five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04).

28. The method of claim 25, wherein the provided cell line is modified to overexpress a sialyltransferase.

29. The method of claim 25, wherein the recovery process comprises subjecting the monoclonal population of binding molecules to in vitro glycoengineering, wherein the in vitro glycoengineering comprises contacting the monoclonal population of binding molecules with a soluble sialyltransferase and a sialic acid substrate.

30. The method of claim 29, wherein the sialyltransferase comprises a soluble variant of human beta-galactoside alpha-2,6-sialyltransferase 1 (ST6GAL1) (SEQ ID NO: 3) and/or the sialic acid substrate comprises cytidine monophosphate (CMP)-N-acetyl-neuraminic acid (CMP-NANA).

31. The method of claim 29, wherein the mass ratio of binding molecule: sialic acid substrate is about 1:4 to about 40:1 and/or the mass ratio of binding molecule:
sialyltransferase is about 80:1 to about 5000:1.

32. The method of any one of claims 29 to 31, wherein the contacting of the monoclonal population of binding molecules with the soluble sialyltransferase and the sialic acid substrate comprises at least 30 minutes of contact at about 2° C. to about 40° C.
